(19) United States
(12) Patent Application Publication
(10) Pub. No.: US 2017/0049848 A1
(43) Pub. Date: Feb. 23, 2017

(54) Target for Firmicutes and Related Bacteria: The PRP Protease

(71) Applicant: VIRGINIA COMMONWEALTH UNIVERSITY, Richmond, VA (US)

(72) Inventors: Gail E. Christie, Richmond, VA (US); Priyadarshan Damie, Richmond, VA (US); Erin A. Wall, Richmond, VA (US); Terje Dokland, Richmond, VA (US); Anton Poliakov, Richmond, VA (US)

(21) Appl. No.: 15/308,455

(22) PCT Filed: May 6, 2015
(86) PCT No.: PCT/US15/29367

§ 371 Date: Nov. 2, 2016

Related U.S. Application Data
(60) Provisional application No. 61/989,330, filed on May 6, 2014.

(51) Int. Cl. A61K 38/08; A61K 45/06; C07K 14/81; C12Q 1/37

(52) U.S. Cl. CPC A61K 38/08; C12Q 1/37; A61K 45/06; C07K 14/8139; G01N 2500/02; G01N 2333/952

(57) ABSTRACT

L27 in Staphylococcus aureus and other Firmicutes is encoded with an N-terminal extension that is not present in most Gram-negative organisms and is absent from mature ribosomes. We have identified a cysteine protease, conserved among bacteria containing the L27 N-terminal extension, which performs post-translational cleavage of L27. The provided methods have utility for the development of new therapeutic antibiotics that target this novel pathway in order to kill pathogenic Firmicutes and related bacteria.

### CROSS REFERENCE TO RELATED APPLICATIONS

This application claims priority to U.S. Provisional application 61/989,330 filed on May 6, 2014.

### FIELD OF THE INVENTION

Embodiments of the invention relate to a novel method of killing Firmicutes and related bacteria by targeting the post-translational cleavage of L27 by Prp Protease.

### BACKGROUND OF THE INVENTION

Staphylococcus aureus is a Gram positive coccus which is both a natural commensal of the human skin and nares and a dangerous pathogen. S. aureus is the most common cause of community acquired skin and soft tissue infections (Morgan, 2011) and the leading cause of hospital acquired infection (Klein, 2007). This versatile bacterium can cause osteomyelitis, septic arthritis, necrotizing pneumonia and toxic shock; severe infection is possible at nearly every location in the body. S. aureus is also the leading cause of infectious endocarditis in North America (Murdoch, 2009). In a study of one thousand individuals with infectious endocarditis, S. aureus was the most common pathogen associated with vegetations on pacemakers and native or prosthetic valves (Leone, 2012). In a separate study by the American Heart Association, S. aureus accounted for 60 to 80% of infections of cardiovascular implantable electronic devices.

The eubacterial ribosome has historically been a prime target for numerous antibiotics, including macrolides (e.g. erythromycin), lincosamides, oxazolidinones and tetracylines, which work by blocking aminoacyl-tRNA binding, preventing peptidyl transfer or causing premature peptidyl-tRNA release (Auerbach, 2002; Yonath, 2005; Colca, 2003; Tejedor, 1986). Though these drugs are beginning to fail, protein synthesis and ribosomal formation remain extremely important yet underdeveloped targets in those bacteria that are only distantly related to the bacterial model organism Escherichia coli.

The protein components of the bacterial ribosome play a large role in efficient ribosomal assembly and function. Ribosomal proteins (r-proteins) are among the most universally conserved cellular components—about half of them occur in all three domains of life, while some remain domain-specific. The study of r-proteins is complicated by nomenclature, and recent efforts have been undertaken to change the naming system to clarify homologous proteins in Archaea, Eukaryota and Bacteria (Ban, 2014). In general, if an r-protein is incorporated into the 50S/60S or “large” subunit, it will be designated Lx where x is an integer. The integer only represents the order in which the r-protein was found, it has nothing to do with size or function. 30S/40S or “small” subunit proteins are labeled Sx in the same way. A major source of confusion comes from non-homologous ribosomal proteins that have been given identical names in each domain of life, e.g. the protein known as L1 in bacteria and yeast has been called L10A in humans, and bears no homology to human L1 (Ban, 2014). It is important to note this fact when exploring the distribution of ribosomal proteins in bacteria. Despite the novel nomenclature proposed by Ban et al, recently it has been the practice to append the letter “a,” “e,” or “p” to the end of the ribosomal protein designation to indicate whether the mentioned protein occurs in Archaea, Eukaryota or Prokaryotes, respectively. Sometimes instead of “p,” “b” will be used instead to indicate Bacteria.

### SUMMARY OF THE INVENTION

Described herein are new findings regarding fundamental S. aureus ribosomal function that have lead to new strategies for antibiotic development against Staphylococci and related pathogens. We have therefore discovered a novel N-terminal extension of L27 in Firmicutes and related bacteria, and this extension must be cleaved away by the newly discovered Prp protease in order for the Staphylococcus aureus to survive. This is a completely unprecedented essential pathway in S. aureus that represents a new target for antibiotic design.

One aspect of the invention provides a method of inhibiting the growth of Firmicutes and related bacteria comprising the step of exposing said bacteria to an effective amount of at least one inhibitor of L27 cleavage. In exemplary embodiments, the bacteria comprises a consensus sequence X₁-X₂-Q-X₃-X₄-A-X₅-K-K (SEQ ID NO:17), wherein X₁ is N or D, X₂ is L or I, X₃ is L, F, or H, X₄ is L or F, and X₅ is H, S, or T. The at least one inhibitor may be a Prp inhibitor selected from the group consisting of a dominant negative inhibitor, a protease inhibitor, a competitive inhibitor, a covalent inhibitor, a conformational inhibitor, a non-competitive inhibitor or an uncompetitive inhibitor. In some embodiments, the dominant negative inhibitor is Prp C34S or Prp C34A. The competitive inhibitor may be a substrate analog. In exemplary embodiments, the substrate analog has an affinity more than one order of magnitude greater than L27. The substrate analog may comprise a non-hydrolyzable peptidomimetic. The non-hydrolyzable peptidomimetic may further comprise one or more modified amino acid residues, beta-linked amino acids, and D-amino acids.

In some embodiments, the Firmicutes or related bacteria are present in a non-human animal. In other embodiments, the Firmicutes or related bacteria are present in a human. The step of exposing may be carried out by intravenous, cutaneous, oral, intraperitoneal, intrathecal, or inhaled administration. The Firmicutes or related bacteria may be pathogenic organisms selected from the group consisting of Staphylococcus, Bacillus, Clostridium, Mycoplasma, Fusobacteria, Listeria, Streptococcus, and Enterococcus. Further embodiments of the invention provide a composition comprising Prp C34S or Prp C34A and a pharmaceutically acceptable carrier.

Another aspect of the invention provides an isolated peptide comprising a sequence selected from the group consisting of SEQ ID NOs: 1-18 or a fragment thereof conjugated to a fluorophore and a quencher. In some embodiments, the isolated peptide is KLNLQFFASKK (SEQ ID NO: 1). In exemplary embodiments, the fluorophore is conjugated to the N-terminus and the fluorophore may comprise 2-amino benzoic acid. The quencher may be conjugated to the C-terminus and the quencher may be dinotrophenol.

Further aspects of the invention provide a method for assaying Prp enzyme activity comprising the steps of contacting Prp with an isolated peptide comprising a sequence selected from the group consisting of SEQ ID NOs: 1-18 or a fragment thereof conjugated to a fluorophore and a quencher; measuring the level of fluorescence; and determining Prp enzyme activity based on the measured level of fluorescence. In some embodiments, the isolated peptide is KLNLQFFASKK (SEQ ID NO:1).

Additional aspects of the invention provide a kit comprising an isolated peptide comprising a sequence selected from the group consisting of SEQ ID NOs: 1-18 or a fragment thereof conjugated to a fluorophore and a quencher. In some embodiments, the isolated peptide is KLNLQFFASKK (SEQ ID NO:1). The kit may further comprise instructions for use and packaging.

Some embodiments of the invention provide a method of screening for compounds that inhibit Prp comprising the steps of adding a test compound to a solution comprising Prp and an isolated peptide comprising a sequence selected from the group consisting of SEQ ID NOs: 1-18 or a fragment thereof conjugated to a fluorophore and a quencher; measuring the level of fluorescence; comparing the level of fluorescence to a reference value; and determining the extent of Prp inhibition based on said comparison. In some embodiments, the isolated peptide is KLNLQFFASKK (SEQ ID NO:1).

### BRIEF DESCRIPTION OF THE FIGURES

FIG. 1. The translating bacterial ribosome uses ribosomal protein L27 to increase tRNA binding efficiency. Normal 50S and 30S subunits assemble independently and translate mRNA using tRNAs charged with amino acids to form a nascent peptide. Charged tRNAs enter at the A site, undergo peptidyl transfer to accept the nascent peptide, move to the P site, release the nascent peptide to the next A site tRNA and then exit from the E site. L27 is located between the A and P sites, where it stabilizes incoming tRNAs and peptidyl transfer. The nascent peptide passes through a tunnel in the 50S subunit and remains attached to a tRNA until the chain is terminated via release factor hydrolysis. In the absence of L27, the 50S subunit assembles incorrectly and translation efficiency is decreased due to impaired A site and P site tRNA binding.

FIG. 2. Staphylococcal phage 80α proteins are processed by a host protease not present in E. coli. This is a composite gel of 80α scaffold and capsid protein expression adapted from Spilman et al, 2012. The lane on the left contains molecular weight markers in kDa, the S. aureus lane shows the protein bands that were produced when 80α scaffold and capsid were expressed in S. aureus in the absence of any other phage proteins. The right lane shows the results of 80α scaffold and capsid expression in E. coli. The bands in the right lane represent un-processed proteins, where no cleavage has occurred. The downward-shifted bands in the S. aureus lane have been N-terminally cleaved at a conserved motif, confirmed by mass spectrometry.

FIG. 3. The 80α scaffold and capsid proteins share a conserved cleavage motif with L27 in Staphylococcus aureus and Bacillus subtilis but not L27 in E. coli. ClustalW alignment of the N-termini of L27 from E. coli (SEQ ID NO:19), B. subtilis (SEQ ID NO:20) and S. aureus (SEQ ID NO:21) with those of bacteriophage 80α scaffold and capsid proteins (GI:148717888; SEQ ID NO:22 and GI:148717889; SEQ ID NO:23, respectively). A dotted line separates the N-terminus encoded in the genome from that found in the mature protein.

FIG. 4. Gibson assembly of a plasmid that replaces native L27 in S. aureus with a spectinomycin resistance cassette—pEW68. It is possible to produce a complete allelic exchange vector (or any multi-piece construction) from PCR products made with overlapping sequence. Primer EAW45, for example, has 15 base pairs of 5′ overlap identical to BamHI cut pMAD, and EAW196 has 15 base pairs of 5′ overlap to the next primer, EAW197, which will link each segment of DNA when the reaction is complete. Plasmid pEW68 is a typical allelic exchange vector that contains 5′ and 3′ flanking homology to the gene that will be replaced, in this case L27.

FIG. 5. S. aureus L27 is cleaved at the conserved motif when it is expressed in its native host but not when expressed in E. coli. Western blot analysis of L27 N-terminal cleavage. Plasmid-encoded S. aureus L27 carried C-terminal Myc and His₆ tags; the blot was probed with anti-Myc antibodies. In the lane labeled Eco, L27 was produced from pEW5 in E. coli BL21. In the lane labeled Sau: L27 was produced from pEW7 in S. aureus SA178RI. L27 in the Sau lane has undergone specific cleavage at the conserved motif to yield the N-terminus ASKK, confirmed by Edman degradation.

FIG. 6. Phylogenetic tree of L27 shows that upstream gene ysxB (Prp) is co-conserved with the N-terminal cleavage motif. L27 phylogeny across major bacterial phyla. This neighbor-joining phylogenetic tree consists of the sequences of ribosomal protein L27 from representative species of each major bacterial phylum. Species containing the conserved N-terminal extension of L27 are italicized. Species containing a ysxB/DUF464/Prp homolog are indicated by diamonds. All species containing the L27 N-terminal extension also contain a Prp homolog. Genbank accession numbers: A. colombiense-ADE56976, C. trachomatis-P66123, C. difficile-Q18B22, M. pneumoniae-P75458, E. rhusiopathiae-YP_004561011, L. johnsonii-Q74IL5, E. faecium-AFK58391, S. pneumoniae-Q04KI2, B. subtilis-CAB14754, S. aureus-NP_374758, H. pylori-YP_006934224, F. nucleatum-WP_005917003, C. aggregans-B8G6X2, L. ferroxidans-WP_014448398, T. lettingae-ABV34531, D. thermophilum-B5YEQ2, A. aeolicus-067650, C. exile-YP_005473593, P. marinus-Q7V0C1, D. acetiphilus-ADD66838, P. mikurensis-BAM03335, N. gonorrhoeae-AAW90302, B. burgdorferi-WP_002656482, T. indicus-YP_004625725, B. fragilis-BAD47759, D. radiodurans-Q9RY65, A. capsulatum-C1F354, C. crescentus-Q9ABB3, C. diphtheriae-YP_005165478, D. indicum-YP_004113510 E. coli-AAC76217, P. ingrahamii-A1SSB5.

FIG. 7A-B. YsxB (Prp) contains conserved catalytic residues His and Cys in a pocket. (A). ClustalW alignment of YsxB sequences from S. aureus (Sau; GI:777227755; SEQ ID NO:24), B. subtilis (Bsu; GI:459390614; SEQ ID NO:25), Streptococcus pneumoniae (Spn; GI:15903057; SEQ ID NO:26), Streptococcus mutans (Smu; GI:640030816; SEQ ID NO:27), Aminobacterium colombiense (Aco; GI:502813267; SEQ ID NO:28), Fusobacterium nucleatum (Fnu; GI:19704453; SEQ ID NO:29), Thermotoga lettingae (Tle; GI:157315433; SEQ ID NO:30) and Mycoplasma pneumoniae (Mpn; GI:13508065; SEQ ID NO:31). Identical and conserved residues are outlined. Asterisks designate the proposed active site residues. The secondary structure elements from the crystal structure of S. aureus Prp (PDB ID: 2P92) are shown above the alignment. (B) Molecular surface of the complete S. aureus YsxB monomer model generated using the partial crystal structure of the S. aureus protein (PDB ID: 2P92) and the complete structures of S. pneumoniae (PDB ID: 2IDL) and S. mutans (PDB ID: 2G0J) as templates. The active site Cys and His residues are indicated by arrowheads.

FIG. 8A-B. Prp is a cysteine protease that cleaves L27 at the conserved motif; Prp C34S mutant is unable to cleave L27 despite D/N residue at the catalytic core. (A) Western blot analysis of L27 N-terminal cleavage. Plasmid-encoded S. aureus L27 carried C-terminal Myc and His₆ tags; the blot was probed with anti-Myc antibodies. Lane “Eco”: L27 produced from pEW5 in E. coli BL21; lane “Sau”: L27 produced from pEW7 in S. aureus SA178RI; lane “Eco+Prp”: L27 co-expressed with Prp in E. coli BL21 (pEW5+pEW15); lane “Eco+PrpC34A”: L27 co-expressed with inactive Prp mutant C34A in E. coli BL21 (pEW5+pEW28). L27 in lanes Sau and Eco+Prp has undergone specific cleavage at the conserved motif to yield the N-terminus ASKK, confirmed by Edman degradation. (B) Western blot (anti-Myc) of S. aureus L27MycHis₆ produced alone in E. coli BL21 (lane 1), co-expressed with Prp C34S (lane 2, after a space), co-expressed with Prp C34A (lane 3) and co-expressed with wild-type Prp (lane 4). In lanes 1-3 L27MycHis₆ remains un-cleaved, while in the presence of wild-type Prp in lane 4, it exhibits the characteristic 1 kDa downward shift that represents specific N-terminal cleavage between P1 Phe 9 and P1′ Ala 10. This was verified by Edman degradation.

FIG. 9. Prp C34A co-purifies with L27MycHis6. Coomassie-stained 16.5% Tris-Tricine gel (Biorad) of L27MycHis₆ co-expressed with Prp C34A (lane 1) and purified Prp (lane 2). The top band in lane 1 is Ni-NTA purified S. aureus L27MycHis₆. The identity of the lower band in lane 1 was confirmed to be Prp by mass spectrometry.

FIG. 10. Prp cleavage of phage 80α scaffold and major capsid proteins. Capsid-like structures formed upon expression of gp46 and gp47 from plasmid pPD2 in the absence (lane 2) and presence (lane 1) of pEW15, expressing Prp, were purified on sucrose gradients and resolved by SDS-PAGE. Lane 3 shows the two samples mixed together, in order to better illustrate the small shift in electrophoretic mobilities of the two proteins following cleavage. M is the protein ladder with selected sizes indicated next to the gel. A MALDI spectrum of the purified capsid fraction run in lane 1 showed major peaks at 21,704 Da and 35,042 Da, corresponding to the masses of cleaved gp46 (theoretical mass 21,701 Da) and cleaved gp47 (35,063 Da), respectively. Tryptic fragments of these two protein bands were also analyzed by LC-MS to confirm that they corresponded to the expected cleaved proteins.

FIG. 11. Growth effects of Prp C34A over-expression. Growth curves of SA178RI containing empty T7 expression vector pG164 (WT), or derivatives expressing wild-type Prp (pEW25) or Prp C34A (pEW26). Bacteria were grown in the presence of inducer and appropriate antibiotics in 96-well format at 32° C. Starting culture density was between 0.12 and 0.15 absorbance at 600 nm, and absorbance was measured every 15 minutes for 16 hours using a microplate reader. Error bars represent a 95% CI, n=3.

FIG. 12. Ribosome assembly profile with dominant negative mutant Prp C34A. Typical sucrose gradient profiles of ribosomal subunits are shown with the positions of expected subunits indicated. The accumulation of what appears to be a pre-50S subunit in the Prp C34A strain indicates that a ribosomal assembly bottleneck has occurred.

FIG. 13. L27 F8A-F9A is un-cleavable. S. aureus L27 and an L27 cleavage motif mutant in which F8 and F9 were replaced with alanines were C-terminally tagged with Myc and His₆ and expressed in S. aureus strain SA178RI. The Western blot shows tagged proteins post purification probed with anti-Myc antibodies. Lane 1, wild-type L27; lane 2 L27 F8A-F9A. The characteristic 1 kDa difference between cleaved and un-cleaved is evident.

FIG. 14. Cell growth following overexpression of L27 mutants. S. aureus strains overexpressing L27 (L27), pre-cleaved L27 (PC), un-cleavable L27 F8A-F9A (UC) or containing empty vector (WT) were grown under inducing conditions in a microplate reader at 32 degrees C. until stationary phase. Optical Density at 600 nm was measured once every 15 minutes. Error bars represent one standard deviation above and below the mean, n=3. There is a significant difference in growth between L27 and UC from t=10 hours to 1=13:30.

FIG. 15. Polysome analysis for strains expressing L27 and L27 mutants. Typical sucrose gradient profiles are shown. The effect of L27 mutant overexpression on ribosomal subunit proportions did not deviate significantly from empty vector. S. aureus strains overexpressing L27 (L27), pre-cleaved L27 (PC), un-cleavable L27 F8A-F9A (UC) or containing empty vector (WT) were grown under the same conditions as those in FIG. 12.

FIG. 16. Growth effects of overexpressing the peptide MLKLNLQFF (GI:749152598). S. aureus strains overexpressing L27 (L27), L27 residues 1-9 as a peptide (Peptide) or containing empty vector (WT) were grown as above. Expression of the peptide caused a deformation in the growth curve at t=3:20 that is not seen in WT and L27. Growth is significantly different between L27 overexpression and peptide overexpression from t=8:20 to t=10:30. Error bars represent one standard deviation above and below the mean, n=3.

FIG. 17. Ribosomal peptide ratios reflect stoichiometric differences in the r-proteins in aberrant vs wild type 50S subunits. Relative change in abundance of ribosomal proteins in aberrant particles. Precursor ion spectra for two tryptic peptides were assessed for each ribosomal protein represented. The absolute areas of each tryptic peptide were compared to those of an early assembly protein within their sample, e.g. the value of (area of peptide 1 from ribosomal protein x/area of ribosomal peptide 1 from L3) was calculated separately for wild type and aberrant particles. Those values were then compared (aberrant value/wild type value) to assess whether the aberrant value represented a significant change compared to that of the wild type. The dotted line represents a ratio of 1, while the continuous line represents a ratio of 0.5. In this graph, the mean of four peptide comparisons for each ribosomal protein (six for L16) is depicted with error bars representing one standard deviation above and below the mean. Most values are extremely close, resulting in error bars that encompass the ratio of 1, displaying no significant change. The peptide values have decreased the most are those of L2, L14, L18, L20, L16, L27 and L25. Not depicted: L24, L32, L33, and L36, for which only one representative peptide could be detected in both wild type and aberrant particle samples. Not detected: L9, L28, L34 and L35.

FIG. 18. Approximate 2D locations of large subunit ribosomal proteins that were lost in conspicuous amounts in the presence of the Prp C34A mutant. Taking into consideration the “semi-quantitative” nature of the data, r-proteins L2, L14, L16, L18, L20, L25, L27, L29, L33a and L36 were represented in MS 1 peptide spectra as having their ratio to early proteins L3 or L4 decreased by at least two fold in the aberrant particle versus a wild type 50S particle.

FIG. 19. Prp and un-cleaved L27 are assembled into aberrant 50S particles. Detection of L27 and Prp in ribosomal particle fractions by Western blot. Aberrant and wild type 50S particle fractions were concentrated and run out on a 16.5% Tris-Tricine gel for maximal separation of the small proteins. The proteins were electroblotted onto PVDF and probed with custom antibodies to L27 and Prp. Both blots show wild type particles on the left (WT) compared to aberrant particles produced in the presence of PrpC34A on the right (PrpC34A).

FIG. 20. ST360 creation—L27 complementation with pEW27 allowed pEW68 to exchange chromosomal L27 with a spectinomycin resistance cassette. In order to replace chromosomal L27 with a spectinomycin resistance cassette, all cultures had to be grown in the presence of 1 mM IPTG to induce expression of L27 from pEW27. This produced sufficient amounts of L27 so that chromosomal L27 could be replaced. ST360 is completely IPTG dependent, because it can no longer produce L27. This demonstrates definitively for the first time that L27 is essential in S. aureus.

FIG. 21. Transformation of ST360 with pT104-derived plasmids pEW72-75 allowed complementation analysis with mutant forms of L27. Each pT104-derived plasmid contains a tightly controlled arsenite-inducible promoter, pictured on the right in each cell with labeled P_{ARS} promoter. Plasmid pEW72 produces wild type L27 with its native cleavage motif (MLKLNLQFF′ASKK; SEQ ID NO:32) (GI:749152598). Plasmid pEW73 produces L27 without the cleavage motif, what we call “pre-cleaved” L27 (M′ASKK . . . ; SEQ ID NO:33). Plasmid pEW74 produces L27 with a motif that is not a Prp substrate (MLKLNLQAAASKK; SEQ ID NO:34). Plasmid pEW75 produces pre-cleaved L27 from one ORF, and then the post-cleavage peptide (MLKLNLQFF; SEQ ID NO:35) from another ORF under the same promoter but with a separate RBS.

FIG. 22A-B. Complementation schematic; only wild type L27 can complement the chromosomal L27 deletion. (A) outline and of the complementation system created in SA178RI for this study. (B) Growth of cells in the presence of different L27 mutants. On the left there is a description of the L27 allele carried on the arsenite-inducible plasmid, forming horizontal rows in this array. On the right, the three columns are photographs taken of solid culture media containing 1 mM IPTG, no inducer, or 5 μM NaAsO₂, pictured in that order. In each row the same amount of each described strain has been spotted onto the different media. Only the strain containing wild type L27 under P_{ARS} can grow on arsenite media. MLKLNLQFF′ASKK=SEQ ID NO:32, M′ASKK=SEQ ID NO:33, MLKLNLQAAASKK=SEQ ID NO:34, and MLKLNLQFF=SEQ ID NO:35.

FIG. 23. L27 depletion results in a severe growth deficit. Cells were first grown to Klett=30 in IPTG media. A 0.5 mL aliquot of the exponential culture was harvested and washed twice with media that did not contain IPTG. These cells were resuspended in 0.5 mL BHI broth and then inoculated into 9.5 mL BHI with or without IPTG. These cultures were shaken at 200 rpm, 37° C. and were measured at hour time points for ten hours. The final sample measurements are above K=300 in the presence of IPTG, and around K=50 without IPTG. Error bars represent one standard deviation above and below the mean, n=3.

FIG. 24. Growth curves of complementation strains grown in arsenite with trendlines. Cells were first grown to Klett=30 in IPTG media. A 0.5 mL aliquot of the exponential culture was harvested and washed twice with media that did not contain IPTG. These cells were resuspended in BHI broth containing 5 □M arsenite (AsO2) and then inoculated into 9.5 mL arsenite BHI broth. These cultures were shaken at 200 rpm, 37° C. and were measured at hour time points for 12-13 hours, and then one measurement was taken at 24 hours. The “Power” trendlines represent the best-fit exponential equation for the data sets of each experiment. Error bars represent one standard deviation above and below the mean, n=3.

FIG. 25. Mechanism of a cysteine protease containing a His/Cys catalytic dyad. Cysteine proteases make use of the fact that thiolate is a strong nucleophile, and that the imidazole group of His can readily abstract a proton from the Cys sulfhydryl group to produce a thiolate moiety. This mechanism involves the formation of an oxyanion tetrahedral intermediate, a covalent acyl-enzyme complex, and then another oxyanion tetrahedral group before hydrolysis of the covalent substrate-enzyme complex is achieved.

FIG. 26. Existing Prp structures share conserved residues and a non-catalytic conformation of the active site. S. aureus (SEQ ID NO:24), S. mutans (SEQ ID NO:27), S. pneumoniae (SEQ ID NO:26), and T. maritima (SEQ ID NO:36) are shown. Highly conserved residues according to the sequence alignment are labeled and mapped onto the ribbon backbone (top left) and depiction of S. pneumoniae Prp dimer (PDB ID 2IDL, top right). This structure represents the entire protein without sequence gaps. The catalytic pair His 22 and Cys 34 are shown in atomic representation on both models. They are oriented in a non-catalytic conformation (see ribbon structure) and obviously not solvent-exposed (see cpk structure). Several of the conserved residues appear to be involved in substrate stabilization near to the catalytic center.

FIG. 27. Ribbon model of S. aureus Prp active site residues in catalytic conformation. The missing residues of PDB ID 2P92 are modeled in a loop that arcs over the catalytic center. This right active site in this model contains the pro-catalytic ionization of active site Cys and His residues, while the left active site residues are in pre-ionization conditions (both shown in atomic representation). The catalytic dyads are now oriented correctly and solvent exposed so that the enzyme/substrate reaction that begins the protease mechanism could be possible.

FIG. 28. Surface model of S. aureus Prp docked with Ac-NLQFFAS-Am.

FIG. 29. Nucleophilic attack by Cys 34 and substrate stabilization by Ser 38. The substrate is depicted with a ribbon backbone, while Prp is modeled in semi-transparent representation with a ribbon backbone. The thiolate ion of Cys 34 attacks the carbonyl carbon of the scissile bond between P1 Phe and P1′ Ala in the substrate. In this model the completely conserved hydroxyl one helix turn below the catalytic Cys 34, Ser 38 in this model, appears to stabilize the amide nitrogen between the P2 Phe and P1 Phe by hydrogen bonding. This could allow the P1 Phe to form π-π stacking interactions with catalytic His 22.

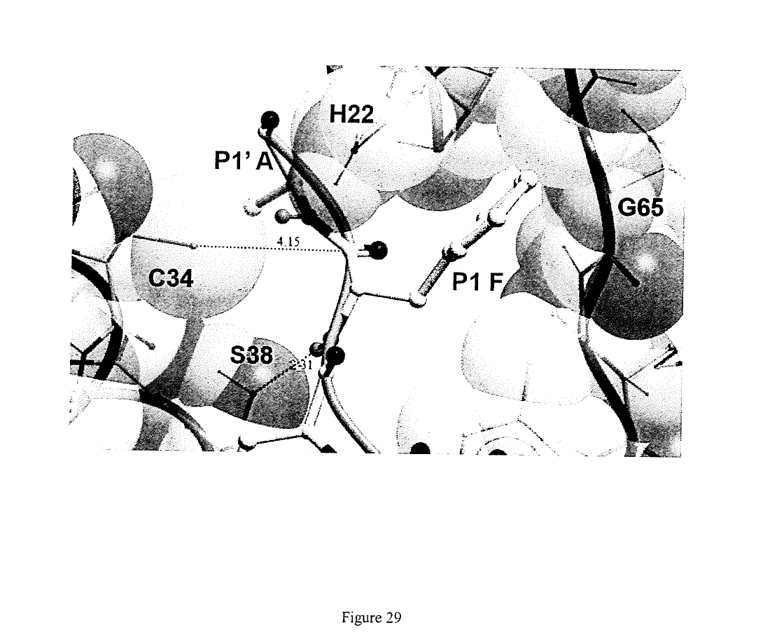

FIG. 30. Putative oxyanion hole formed by amides of Gly 21, Gly 65 and the non-conserved subsequent residue His 66. The amide nitrogens of Gly 21, Gly 65 and His 66 (not residue specific) likely flex in solution to support the oxyanion formed during Prp catalysis. Gly 21 and Gly 65 are completely conserved, indicating intrinsic flexibility necessary in this region. These two residues lie in the same plane as the nucleophilic attack, making their location ideal to receive the reactive oxyanion moiety.

FIG. 31. Quenched fluorescent peptide assay mechanism. A properly designed peptide substrate with a fluorophore on one terminus and a quencher on the other will not fluoresce until it is cleaved. Monitoring fluorescence evolution at the appropriate wavelength allows performance of a continuous assay, useful in determining enzyme kinetics.

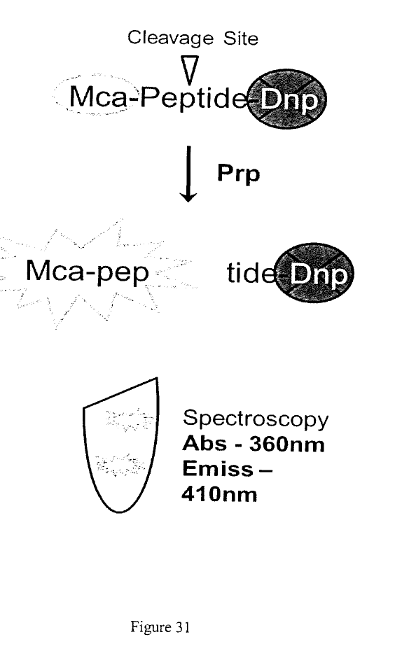

FIG. 32. Only active tagged Prp will cause fluorescence increase in the presence of quenched peptide substrate. Wild type Prp, mutant Cys 34 Ala and mutant Cys 34 Ser were assayed as N-terminal His-SUMO fusions. Only wild-type Prp cleaved the peptide substrate to release the fluorophore and caused relative fluorescence increase.

FIG. 33. L27 cleavage logo compared to FlhB N-terminal sequence logo. Hidden Markov Model (HMM) logo representing alignments of N-terminally extended L27 and FlhB homologs in bacteria that contain Prp.

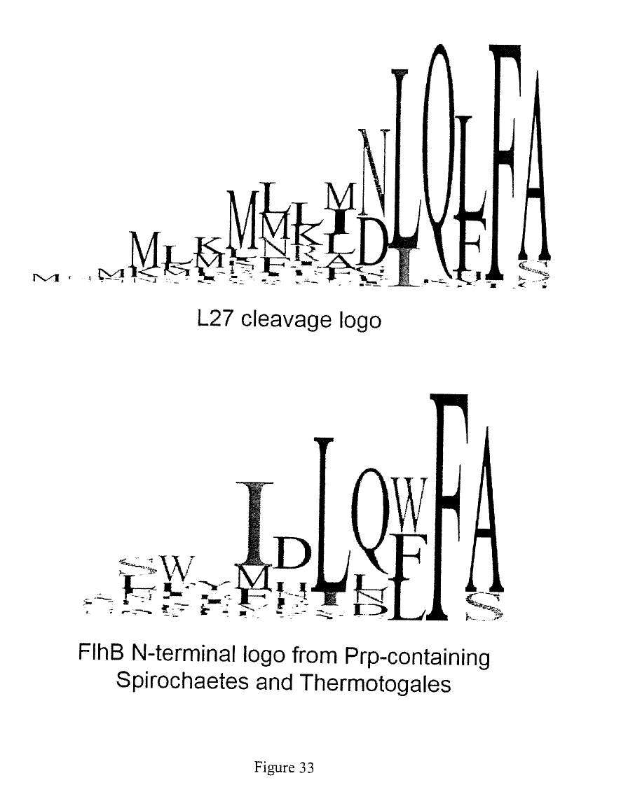

### DETAILED DESCRIPTION OF THE INVENTION

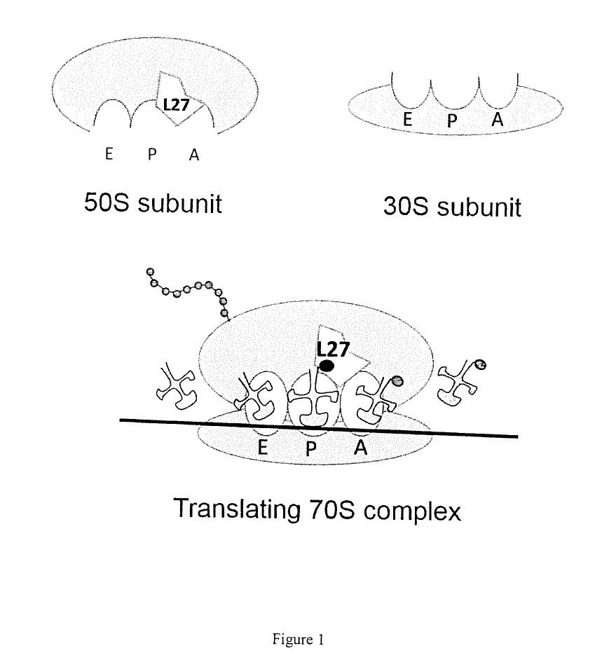

Ribosomes, the molecular machines that carry out protein biosynthesis, are ribozymes composed of ribosomal RNA (rRNA) and proteins. Ribosomes consist of two ribonucleoprotein complex subunits, one small and one large. The subunits and assembled ribosome are designated by their sedimentation rate—the small 30S and large 50S in bacteria; a complete ribosome containing both subunits is 70S. The large and small subunit each contain part of the A, P and E sites through which transfer RNAs (tRNAs) charged with amino acids enter to add their residue to the nascent peptide chain, are deacylated, and exit, respectively (FIG. 1). A “ratcheting” motion occurs due to the independent movement of the small and large subunits such that a tRNA can, for example, occupy the A site on the small subunit and the P site on the large subunit. The hybrid states, in order, are A/A, A/P, P/P, P/E, E/E, with the site occupied on the 30S subunit noted first. In this way the movement of the two subunits, fueled by GTP, aids the passage of the tRNAs (Frank, 2000). The nascent peptide chain is continually polymerized as the C-terminal end is transferred to the P/P site tRNA after each peptide bond formation at the A/P hybrid site. The 16S rRNA forms the 30S subunit and contains the decoding center where mRNA codons are paired with the appropriate anticodons on incoming charged tRNAs. The 23S rRNA forms the major nucleic acid component of the large subunit and is complexed with the 5S rRNA that forms the central protuberance of the ribosome. The 50S subunit contains the Peptidyl Transferase Center (PTC) on Domain V of the 23S rRNA, and the exit tunnel through which the nascent peptide must pass. The PTC is the location of the active site nucleotides that catalyze peptide bond formation and tRNA de-acylation. Here we explore in detail the bacterial PTC components—rRNA and ribosomal proteins.

In bacteria, the peptidyl transferase center (PTC) is composed largely of rRNA, with only very few r-protein components in close proximity. Universally, the PTC is positioned within a pseudo two-fold symmetrical region which contains conserved nucleotides that catalyze protein formation in two ways. First, there are nucleotides that seem to assist in catalysis of peptide bond formation through a proposed “proton wire,” composed of coordinated water molecules, that stabilizes the nucleophilic attack by the incoming residue on the ester bond between the P-site tRNA and the nascent peptide chain (Polikanov, 2014). Second, there are nucleotides that contribute entropically through induced-fit conformational changes that stabilize and facilitate the A-site tRNA rotatory movement required for substrate-mediated acceleration (reviewed in Bashan and Yonath, 2008). The ribosome has been thought to be a ribozyme for at least the past fifteen years (Cech, 2000). This conclusion was initially drawn based on the crystal structure of the 50S subunit from Archaeon Haloarcula marismortui in which the PTC is composed of rRNA exclusively (Nissen, 2000). However, later high resolution structures of the bacterial ribosome (as opposed to the archaeal) demonstrated the presence of the N-terminal region of a ribosomal protein only eight angstroms from the center of the PTC that participates in the proton wire—ribosomal protein L27 (Voorhees, 2009; Polikanov 2014).

Bacterial ribosomal protein L27, product of the rpmA gene, is a component of the large ribosomal subunit found only in eubacteria and in the ribosomes of mitochondria and chloroplasts. This homology follows the endosymbiont theory—mitochondria and chloroplasts (plastids) are thought to have evolved from small rickettsial bacteria and cyanobacteria, respectively, which became engulfed by larger cells (Sagan, 1967). L27 is highly conserved, and although deletions can be tolerated in some bacterial species (albeit with severe growth defects), rpmA is generally considered an essential gene. L27 consists of a C-terminal β-sandwich domain and a long N-terminal arm that extends into the PTC. Its rRNA contacts include domain II, helix 34 and domain V helix 81 and 86 on the 23S rRNA. It is also in contact with the 5S rRNA, positioned at the central protuberance in the midst of the ribosome (Fox, 2010; helix numbers from Yusupov, 2001). All bacteria seem to contain only a single copy of L27, despite the fact that r-protein paralogs are common, especially in the low GC phylum Firmicutes to which S. aureus belongs (Yutin, 2012).

The role of r-protein L27 in substrate stabilization at the PTC and thus entropic contributions to catalysis has been increasingly appreciated in the last decade (Wower, 1998; Maguire, 2005; Voorhees, 2009; Polikanov, 2014). The new work on the proton wire model implicates the N-terminus of r-protein L27 (after loss of the initial N-formyl methionine) as an extremely important participant in the coordination of a water molecule that supports the proton wire, while also serving as an agent of P-site tRNA stabilization (Polikanov, 2014; Wang, 2012). It has been chemically demonstrated to play a critical role in tRNA substrate stabilization during the peptidyl transfer reaction (Wang, 2004a; Maguire, 2005). The 3′ ends of both A- and P-site tRNAs in the PTC can be cross-linked to L27 in E. coli (Wower, 1998). In a genetic complementation assay, deletion of the first three N-terminal amino acids (A2 H3 K4; the universal fMet is removed) from E. coli L27 resulted in a drastically decreased growth rate, loss of tRNA crosslinking and a defect in peptidyl transferase activity (Maguire, 2005). FRET analysis of peptidyl-tRNA dynamics implicated L27 residue K4 in stabilization of the P-site tRNA (Wang, 2012; Xiao, 2012). Deletion of the entire E. coli rpmA gene led not only to a severe growth defect but also incomplete assembly of the 50S ribosomal subunit, indicating a role for L27 in ribosome assembly (ribosomes lacking L27 also lost r-proteins L16, L20 and L21) as well as catalysis (Wower, 1998). The role of the L27 N-terminus in translation and thus bacterial biology is large and well-studied—any variations in the conserved region would be of considerable importance, and highly worth investigating.

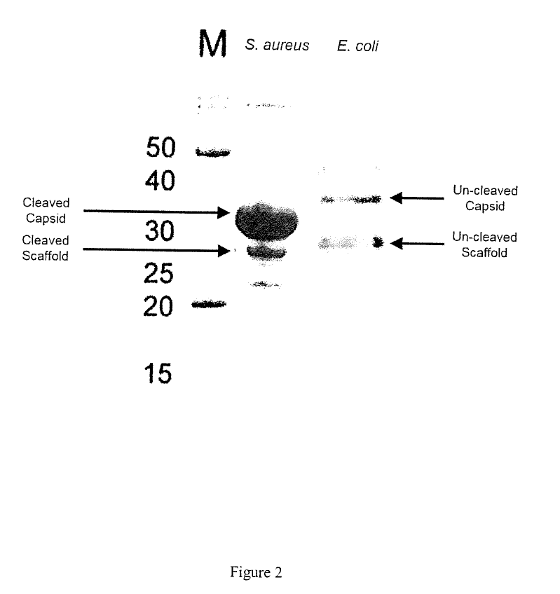

In the course of studying capsid assembly in staphylococcal phage 80α, a cleavage event that occurred in the phage scaffold and major capsid proteins, gp46 and gp47, was examined. It is very common for phage to utilize a “prohead” protease to cleave their scaffold away after procapsid assembly, in order to form a mature capsid. However, 80α does not encode a protein with any predicted homology to known proteases. It was instead found that these proteins were cleaved correctly at their conserved motif upon expression in S. aureus in the absence of any other phage proteins, implicating a host protease in this process (see FIG. 2). Over-expression of the processed versions of both capsid and scaffold proteins in S. aureus led to formation of polyheads, or polymer sheets and tubes of capsid protein. This seemed to indicate that the cleavage of both capsid and scaffold proteins might have regulatory implications during capsid assembly (Spilman, 2012).

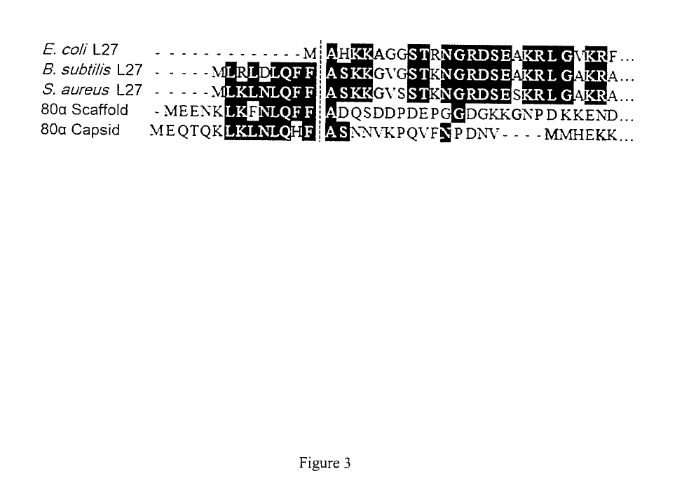

The cleavage did not appear to be autoproteolytic, and no equivalent cleavage of either protein ever occurred upon expression in E. coli. This was the first demonstration of the involvement of a bacterial host protease in bacteriophage assembly. A search for a host protein with a potential homologous cleavage site to that of gp46 and gp47 identified a similar sequence at the N-terminus of S. aureus ribosomal protein L27 (Spilman, 2012). This motif is conserved in all known Firmicutes, including Bacillus, Listeria, Clostridium and Streptococcus. It represents an N-terminal extension that occludes well-studied residues that have been found in the PTC. This motif is not found in bacteria such as E. coli. Importantly, sequence specific cleavage in the Firmicute L27 was predicted to generate an N-terminus identical to that found in E. coli L27 (see FIG. 3).

Several lines of evidence were consistent with this postulated cleavage. Based on the structure of L27 and its position in known ribosome structures (Voorhees, 2009; Polikanov, 2014), the N-terminal extension would reach into the PTC and interfere with the peptidyl transfer reaction via massive steric hindrance. Further, the N-terminal four residues of E. coli L27 constitute a highly conserved A(S/H)KK (GI:85675979; SEQ ID NO:18) motif that is also found in S. aureus L27, immediately following the 9-residue N-terminal extension. Antibiotics that inhibit the peptidyltransferase reaction by obstructing the PTC, such as oxazolidinone, crosslink to S. aureus L27 residues in the A(S/H)KK (GI:749152598; SEQ ID NO:18) motif (Colca, 2003; Leach, 2007) and not to any residues N-terminal to those. These observations suggested that the N-terminal extension is not present in the mature, functioning ribosome.

Consistent with this expectation, no tryptic peptide corresponding to the N-terminal extension was found in ribosomes isolated from S. aureus (Colca, 2003). The Bacillus subtilis rpmA gene also encodes an L27 protein with a similar N-terminal extension, and these extra amino acids are lacking in the L27 protein isolated from B. subtilis ribosomes; this discrepancy had been noted previously, but attributed to misannotation of the gene (Lauber, 2009). Since L27 is positioned at the heart of the ribosomal active site, where it plays a key role in translation (Maguire, 2005), we have further elucidated the role of this N-terminal extension in S. aureus biology.

Described herein is the function and distribution of L27 proteins with this N-terminal extension motif, which immediately precedes and obscures the highly conserved PTC-participating region on the N-terminus of L27 in E. coli. The data presented in the Example demonstrates that this extension is cleaved post-translationally in S. aureus, prior to or concurrent with ribosome assembly. A universally conserved cysteine protease in bacteria containing the L27 N-terminal extension that performs this cleavage has been identified. Both L27 and the protease, which is termed Prp, are essential in S. aureus. This indicates that the cleavage event is essential as well. Prp is also known as YsxB. The recent emergence of virulent, antibiotic resistant strains of S. aureus that spread among otherwise healthy individuals is a considerable public health concern that necessitates new antibiotics. Prp is an effective target for antibiotics specific to Gram-positive bacteria such as S. aureus and other Firmicute or Tenericute pathogens in which this specific L27 processing occurs.

It is an object of the invention to provide methods of preventing, inhibiting, or slowing the growth of Firmicutes and related bacteria. In some embodiments, these methods comprise the step of exposing said bacteria to an effective amount of at least one inhibitor of L27 cleavage or at least one inhibitor of Prp. Such Firmicute bacteria include, but are not limited to, the genera Aerococcus, Bacillus, Clostridium, Coprococcus, Enterococcus, Gemella, Lactobacillus, Lactococcus, Leuconostoc, Listeria, Marinococcus, Melissococcus, Micrococcus, Pediococcus, Peptococcus, Peptostreptococcus, Planococcus, Ruminococcus, Saccharococcus, Salinococcus, Staphylococcus, Streptococcus, Trichococcus, and Vagococcus. In exemplary embodiments, the Firmicutes and related bacteria are selected from the group consisting of Staphylococcus, Bacillus, Clostridium, Listeria, Streptococcus, and Enterococcus. The phylum Fusobacteria contains the same Prp/L27 extension although the group is Gram negative; this group includes the obstetric and dental pathogen Fusobacterium nucleatum. Some pathogenic members of the Tenericutes/Mollicutes also contain this system and would be killed by an antibiotic against Prp. This includes, but is not limited to, human pathogen Mycoplasma pneumoniae and bovine pathogen Mycoplasma bovis.

Inhibitors of L27 cleavage or inhibitors of Prp may fall into the following categories: dominant negative inhibitors, protease inhibitors, competitive inhibitors, noncompetitive inhibitors, covalent inhibitors, conformational inhibitors, and uncompetitive inhibitors. Dominant negative mutants of Prp such as Prp C34A as described in the Example, are effective inhibitors of Prp. Any Prp mutation that abolishes its proteolytic activity while maintaining its binding to L27 may be used as an inhibitor of the invention. Inhibition of Prp with a protease inhibitor kills those bacteria that employ Prp to cleave L27. Prp also represents a unique protein fold, with no significant structural homology to any studied human proteins, which make it a truly novel drug target.

Competitive inhibition via a substrate analog for example is contemplated. In this approach, a non-hydrolyzable peptidomimetic “dummy substrate” containing modified residues, beta-linked amino acids or D-amino acids would inhibit Prp activity. Peptidomimetics may contain covalent “warheads” discussed in the next paragraph, but diminished peptidic character in order to improve their bioavailability and stability. In these compounds the peptide bond is replaced by azapeptides possessing an azaglycine residue, cyclohexanone, phenyl ring, etc. These molecules could bind Prp with an affinity orders of magnitude greater than that of the native target. In some embodiments, the binding affinity of the inhibitor is at least one order of magnitude greater than the native target. Trials on the human administration of peptidomimetics containing D-amino acids and beta linkages suggest that they are not intrinsically toxic (Vlieghe, 2010).

Covalent modification is a very potent mechanism of inhibition. One covalent inhibitor of Prp activity is the alpha-ketoheterocycle covalent modification group used as a key imbuing functionality to peptides or peptidomimetics derived from the substrate cleavage motif [KLNLQFFASKK from GI: 749152598; SEQ ID NO: 1]. It is known that the moderately reactive electrophilic ketone of alpha-ketoheterocycles can form a reversible covalent bond with cysteine and other nucleophiles at the active site. Alpha-ketoheterocycles have successfully led to the development of potent inhibitors for cysteine and serine proteases, transferases, and hydrolases.

Prp can take many non-catalytic conformations, as evidenced by the fact that none of the four Prp crystal structures that exist would be capable of catalysis due to the orientation of their active site residues. In the Prp family, the catalytic His is always preceded by a Gly, forcing it onto a flexible loop that appears to be able to swing outward, away from the catalytic Cys. This has functional relevance in substrate binding and specific catalysis. Therefore conformational inhibitors that target the flexible loop are also effective Prp inhibitors. For example, a tight-binding inhibitor could simply stabilize extant non-catalytic conformations of the enzyme. The inhibitor could sterically hinder the movement of the active site histidine side chain, forcing it away from the catalytic cysteine, as its conformation in structures PDB ID 2G0I and 2IDL.

In a hybrid approach, the covalent enzyme-substrate complex may be stabilized by an uncompetitive inhibitor, which would prevent regeneration of the Prp catalytic center by preventing Prp-L27 dissociation. In this way, the enzyme active site remains covalently modified by its native substrate without the introduction of a reactive warhead drug that could have off-target effects (Deu, 2012). Our studies of catalytically inactive PrpC34A overexpression demonstrated the capacity of a non-covalent L27-PrpC34A complex to radically inhibit S. aureus growth and ribosome production. In light of the rise in antibiotic resistance in S. aureus and other Firmicute Gram-positive pathogens, such as Clostridium difficile and Enterococcus faecium, and the paucity of novel antibiotics in the development pipeline (Gould, 2009; Hughes et al., 2014), the findings reported here provide new therapeutic approaches that interfere with protein synthesis by targeting the processing of L27 by Prp, thus providing a novel mechanism for killing Firmicutes and related bacteria.

The antibiotic agents of the invention (e.g. inhibitors of L27 cleavage or inhibitors of Prp) can be used to prevent, inhibit, or slow the growth of Firmicute, Mollicute and Fusobacterial pathogens both in vitro and in vivo. The terms “subject” and “patient” are used interchangeably herein, and refer to an animal such as a mammal, which is infected with or suspected of having, at risk of, or may be exposed to Gram-positive bacteria. The subject may be a human. The terms also include domestic animals such as horses, cows, sheep, poultry, fish, pigs, cats, dogs, and zoo animals such as goats, apes (e.g. gorilla or chimpanzee), and rodents such as rats and mice.

The antibiotic agents may be administered in vivo by any suitable route including but not limited to: inoculation or injection (e.g. intravenous, intraperitoneal, intramuscular, subcutaneous, and the like), and by absorption through epithelial or mucocutaneous linings (e.g., nasal, oral, and the like). Other suitable means include but are not limited to: inhalation (e.g. as a mist or spray), orally (e.g. as a pill, capsule, liquid, etc.), intranasally, as eye drops, etc. In preferred embodiments, the mode of administration is oral or by injection. In addition, the agents may be administered in conjunction with other treatment modalities such as substances that boost the immune system, additional antibiotic agents, and the like. Antibiotics that may be administered in conjunction with the antibiotic agents of the invention include, but are not limited to, penicillins, penicillin derivatives, cephalosporins, monobactams, carbapenems polymyxins, rifamycins, lipiarmycins, quinolones, sulfonamides, macrolides, lincosamides, tetracyclines, lipopeptides, glycylcyclines, oxazolidinones, and lipiarmycins. In exemplary embodiments, the mode of administration is selected from the group consisting of intravenous, cutaneous, oral, intraperitoneal, intrathecal, and inhaled administration.

The methods of this disclosure involve administering an antibiotic agent to a subject in need of protection from or treatment of a bacterial infection. Such methods typically involve identifying a suitable subject e.g. an individual who has been or is likely to be exposed to a Gram-positive bacteria, and administering a therapeutically effective dose of the antibiotic agent to the subject. In some aspects, what is provided is a method of prophylactically preventing the establishment of a bacterial infection in an individual, e.g. preventing or lessening the development of at least one symptom of a bacterial infection.

In the context of the invention, the term “treating” or “treatment”, as used herein, means reversing, alleviating, inhibiting the progress of, or preventing the disorder or condition to which such term applies, or one or more symptoms of such disorder or condition. A “therapeutically effective amount” is intended for a minimal amount of antibiotic agent which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to a disorder.

The dose of antibiotic agent and the timing and mode of administration varies from individual to individual e.g. based on the type of bacteria; the stage of infection (if an infection is already present); the age, gender, genetic background, and overall general health of the individual, etc., and is best determined by a skilled medical practitioner such as a physician. Generally, the dose will be in the range of from about 1 to about 500 mg/kg of body weight. Frequency of administration generally ranges from 1 to 4 times per day, although slow release formulations may permit administration daily or every few days.

One aspect of the invention provides a high throughput-capable continuous fluorescent assay. Methods of assaying Prp enzyme activity may comprise the steps of contacting Prp with an isolated peptide of the invention conjugated to a fluorophore and a quencher; measuring the level of fluorescence; and determining Prp enzyme activity based on the measured level of fluorescence evolved over time. This assay monitors Prp activity in the presence of a quenched fluorescent peptide containing a cleavage motif of L27. In this assay, when the substrate is cleaved the fluorophore is released from its quenching partner and fluorescence readings at the fluorophore emission wavelength increase with time. There can be low level stable background fluorescence, but the majority of fluorescence should only be evolved in the presence of active Prp. In an exemplary embodiment, the fluorophore 2-amino benzoic acid (2-abz) is conjugated to the N terminus of the peptide, with a dinitrophenol (Dnp) quencher conjugated to the C-terminus.

It is an object of the invention to provide isolated polypeptides and fragments thereof to be used in an assay for Prp activity or as Prp inhibitors. The amino acid sequence of polypeptides of the invention may be altered somewhat and still be suitable for use in the present invention. For example, certain conservative amino acid substitutions may be made without having a deleterious effect on the ability of the polypeptides to induce proteolytic cleavage. Those of skill in the art will recognize the nature of such conservative substitutions, for example, substitution of a positively charged amino acid for another positively charged amino acid (e.g. K for R or vice versa); substitution of a negatively charged amino acid for another negatively charged amino acid (e.g. D for E or vice versa); substitution of a hydrophobic amino acid for another hydrophobic amino acid (e.g. substitution of A, V, L, I, W, etc. for one another); etc. All such substitutions or alterations of the sequences of the polypeptides that are disclosed herein are intended to be encompassed by the present invention, so long as the resulting polypeptides still function to induce proteolytic cleavage.

In some embodiments, the isolated polypeptides contain sequences that are at least about 80% (for example at least about 85%, 90%, 95%, 99%) identical to SEQ ID NOs: 1-18, the N-terminal sequences of L27 from Firmicutes and related bacteria. For example, a cleavage motif on L27 may be used such as KLNLQFFASKK (SEQ ID NO: 1). A consensus sequence of some of the motifs is P6 N/D, P5 L/I, P4 Q, P3 L/F/H, P2 L/F, A, P1 F, P′1 A/S (SEQ ID NO:17). Nomenclature Px indicates a residue before the cleavage site, P′x indicates a residue after the cleavage site. Some exemplary pathogens and their L27 cleavage motifs are shown in Table 1.

TABLE 1
Exemplary pathogens containing the L27 N-terminal extension and a Prp homolog.

| Organism | L27 GI:# | Cleavage sequence | Prp GI:# |
|---|---|---|---|
| Bacillus cereus ATCC 14579 | 39931864 | MLRLDLQFF′ASKK | 30022518 |
| | | (SEQ ID NO: 2) | |
| Clostridium difficile 630 | 123067055 | MLNMNLQLL′ASKK | 123174514 |
| | | (SEQ ID NO: 3) | |
| Clostridium perfringens F262 | 380304037 | MLKMNLQLF′AHKK | 122956552 |
| | | (SEQ ID NO: 4) | |
| Clostridium tetani | 693311599 | MLLMNLQLF′ATKK | 75541967 |
| | | (SEQ ID NO: 5) | |
| Enterococcus faecalis | 703904121 | MLLTMNLQLFAHKK | 81437169 |
| | | (SEQ ID NO: 6) | |
| Enterococcus faecium DO | 388533199 | MLLSMNLQLF′AHKK | 122640342 |
| | | (SEQ ID NO: 7) | |
| Erysipelothrix rhusiopathiae | 509079375 | MKFVLDIQLF′ASKK | 509079376 |
| SY1027 | | (SEQ ID NO: 8) | |
| Fusobacterium nucleatum | 492668073 | MQFLFNIQLFAHKK | 75384271 |
| | | (SEQ ID NO: 9) | |
| Listeria monocytogenes FSL | 378753235 | MLKFDIQHF′AHKK | 386050517 |
| J1-208 | | (SEQ ID NO: 10) | |
| Mycoplasma pneumoniae M129 | 2500314 | MNNKYFLTKIDLQFF′ASKK | 2496339 |
| | | (SEQ lD NO: 11) | |
| Peptostreptococcus stomatis | 495063007 | MLKMNLQLL′ASKK | 495063035 |
| DSM 17678 | | (SEQ ID NO: 12) | |
| Staphylococcus aureus subsp. | 15927225 | MLKLNLQFF′ASKK | 81781470 |
| aureus N315 | | (SEQ ID NO: 13) | |
| Streptococcus pneumoniae D39 | 122278648 | MLKMTLNNLQLF′AHKK | 122278649 |
| | | (SEQ ID NO: 14) | |
| Streptococcus pyogenes M1 GAS | 13621998 | MLK1VINLANLQLFAHKK | 15674861 |
| | | (SEQ ID NO: 15) | |
| Ureaplasma parvum serovar 3 str. | 20139831 | MNKLYWLTDLQLF′ASKK | 81549653 |
| ATCC 700970 | | (SEQ ID NO: 16) | |

L27 Cleavage motif consensus sequence. Amino acids separated by periods-
variation noted by slashes: N/D.L/I.Q.L/F/H.L/F′A.H/S/T.K.K. (SEQ ID NO: 17)
Identification numbers for each protein in Genbank (GI numbers) are noted. The cleavage motif varies in each pathogen, but certain consensus residues can be discerned, noted in the bottom cell. The scissile bond is noted with an apostrophe.

The polypeptides of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques. Polypeptides of the invention can be use in an isolated (e.g., purified) form or contained in a vector, such as a membrane or lipid vesicle (e.g. a liposome). In specific embodiments, it is contemplated that polypeptides according to the invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

Another object of the invention relates to an isolated, synthetic or recombinant nucleic acid encoding for a polypeptide according to the invention. Typically, said nucleic acid is a DNA or RNA molecule, which may be included in any suitable vector, such as a plasmid, cosmid, episome, artificial chromosome, phage or a viral vector. The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Another object of the invention relates to a vector comprising a nucleic acid of the invention. Such vectors may comprise regulatory elements, such as a promoter, enhancer, terminator and the like, to cause or direct expression of said polypeptide upon administration to a subject. The vectors may further comprise one or several origins of replication and/or selectable markers. The promoter region may be homologous or heterologous with respect to the coding sequence, and provide for ubiquitous, constitutive, regulated and/or tissue specific expression, in any appropriate host cell, including for in vivo use. Examples of promoters include bacterial promoters (T7, pTAC, Trp promoter, etc.), viral promoters (LTR, TK, CMV-IE, etc.), mammalian gene promoters (albumin, PGK, etc), and the like.

Another object of the present invention relates to a cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The nucleic acids of the invention may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). More particularly, the invention contemplates vascular or endothelial cells thereof or derived thereof, such as human umbilical vein endothelial (HUVEC) or progenitor endothelial cells (PEC).

Another object of the invention relates to a pharmaceutical composition comprising a polypeptide or nucleic acid according to the invention and a pharmaceutically acceptable carrier. Typically, polypeptide or nucleic acid according to the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

An additional aspect of the invention provides methods of screening for compounds that inhibit Prp. These methods may comprise the steps of adding a test compound to a solution comprising Prp and an isolated peptide of the invention conjugated to a fluorophore and a quencher; measuring the level of fluorescence; comparing the level of fluorescence to a reference value; and determining the extent of Prp inhibition based on said comparison. For example, a decrease in fluorescence as compared to a control sample containing only the Prp enzyme and polypeptide without the test compound is indicative of inhibition of Prp.

A further aspect of the invention relates to kits for performing the methods of the invention, wherein said kits comprise the isolated polypeptides of the invention. The kit may also comprise means for measuring the fluorescence level of the polypeptides of the invention. Kits may typically comprise two or more components required for performing a screening assay. Components include but are not limited to compounds, reagents, containers, and/or equipment. The components may be packaged with the necessary materials into suitable containers. Controls (such as positive and negative controls) can also be included in some kits.

The reference or control values used in the methods of the invention may include positive or negative controls. For example, a positive control of Prp enzyme activity may include a solution containing the Prp enzyme and a polypeptide of the invention, wherein the solution does not contain a Prp inhibitor. The level of fluorescence in this solution may be set as 100% enzyme activity. The positive control for Prp enzyme activity can be used as a negative control for Prp inhibition. Here the level of fluorescence in a solution containing Prp and a polypeptide of the invention (without a Prp inhibitor) may be set as 0% inhibition. Along those same lines, an inhibitor of Prp activity may be used as a negative or positive control depending on the assay and viewed as 0% enzyme activity or 100% inhibition. An effective Prp inhibitor generally decreases enzyme activity or increases inhibition by at least 10%, more preferably by at least 50% or more.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

### Methods

Bioinformatics. The National Center for Biotechnology Information (NCBI) website was the source for most bioinformatics data presented in this work. The Conserved Domain Database (CDD) was where it was first noticed that ysxB was part of a family known only as Domain of Unknown Function (DUF) 464. The CDD and the Wellcome Trust protein family site, Pfam, list species that contain members of any specified protein domain family, and this allowed the determination of which bacteria phyla contained a DUF464 homolog. Members of these phyla were manually inspected to determine whether they contained N-terminally extended L27 using the NCBI Graphics View of each fully annotated genome.

The L27 protein alignment (FIG. 3) was generated through the Mobyle Portal of the Pasteur institute using BOXSHADE v3.31 C (Neron, 2009). A MUSCLE alignment of the five proteins was generated using the CLUSTALW algorithm and default preset values in the bioinformatics software package Geneious (Kearse, 2012). This alignment file was submitted to the Mobyle Pasteur BOXSHADE server. All other alignments were generated in Geneious using the MUSCLE algorithm.

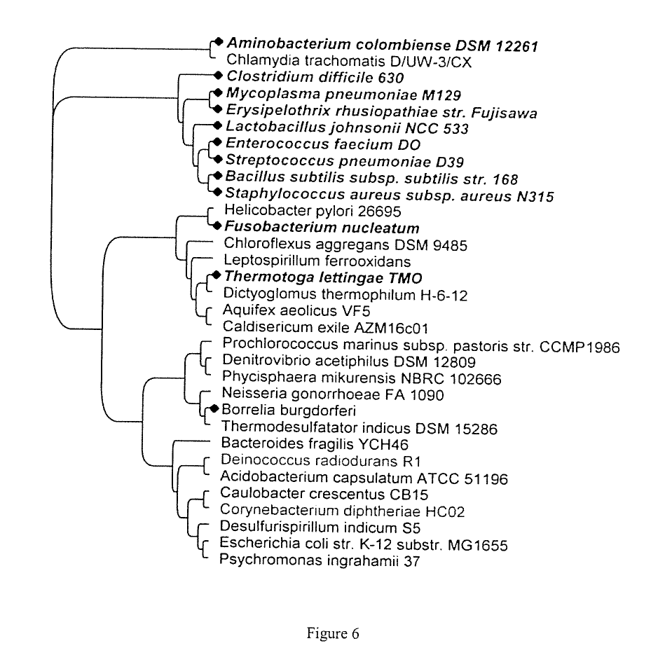

The L27 phylogenetic tree (FIG. 6) was generated in Geneious using the BLOSUM80 cost matrix. The L27 tree is derived from representative L27 protein sequences from each bacterial phylum. These sequence data were subjected to global alignment with free end gaps (gap open penalty set to 12, gap extension penalty set to 3). The genetic distance model utilized was Jukes Cantor and the tree build method was neighbor-joining with no outgroup.

Molecular Modeling. The existing crystal structure of S. aureus Prp (PDB ID: 2P92) lacks the loop that includes the active site residues His 22 and Cys 34. The PDB file of the incomplete Staphylococcus aureus Prp/YsxB homolog dimer was read into SYBYL-X version 2.1. Modeller version 9.12 to was used to model the loop that was not present in the crystal structure. One loop model was created using the loop_model.py script and the loop was refined using the loop_refine.py script. Models were created and read into SYBYL and one was chosen that contained a favorable His/Cys pro-catalytic conformation. The four residue loop (residues 60-63), had a closed conformation that partially obscured the active site of the enzyme. For this reason, the loop_refine.py script was again utilized to allow the four residue loop to adopt a conformation that did not obscure the active site. The model with greatest solvent exposure of the catalytic site was chosen. This model was read into SYBYL, along with the original crystal 2P92 which formed the template for modeling the dimer. This dimer was read into Gold 5.2, and docking was achieved using the blocked peptide Ac-Asn-Leu-Gln-Phe-Phe-Ala-Ser-Am.

Molecular dynamics (MD) simulations were carried out with the NAMD 2.8 package developed by the Theoretical and Computational Biophysics Group in the Beckman Institute for Advanced Science and Technology at the University of Illinois at Urbana-Champaign (Phillips, 2005). CHARMM (Charmm-27) was used as the force field (MacKerell, 1998). Prior to simulation, the ionization states of His residues were checked using PROPKA 3.1 (Olsson, 2011). The analysis of the MD trajectory was done in VMD (Humphrey, 1996).

Bacterial Culture. All S. aureus strains used in this work are derivatives of the phage-cured, restriction-defective strain RN4220 (Kreiswirth, 1983). The S. aureus expression strain SA178RI carries a T7 RNA polymerase expression cassette under lac operator control (D'Elia, 2006). The plasmid used for expression in SA178RI was pG164, an E. coli-S. aureus shuttle vector carrying the T7 late promoter into which a lac operator, a multiple cloning site, an optimized gram-positive ribosome binding site, and a constitutively expressed copy of the lac repressor gene were introduced (D'Elia, 2006). S. aureus strains were grown in Trypticase Soy Broth (TSB) (Remel, Lenexa, Kans.) or Brain Heart Infusion (BHI) (Remel, Lenexa, Kans.) at 32° C.

Cell growth was routinely measured with a Klett-Summerson colorimeter. E. coli strains were grown in Luria Bertani (LB) (Difco, Franklin Lakes, N.J.) medium with appropriate antibiotics at 37° C. at 200 rpm to Klett of 90, then shifted to 32° C. at 200 rpm for protein induction. E. coli strains containing a plasmid with the PBAD promoter were induced using 0.2% arabinose. All strains containing isopropyl β-D-1-thiogalactopyranoside (IPTG)-inducible promoters were induced with 1 mM IPTG (Goldbio; St. Louis, Mo.).

DNA manipulations. Restriction endonucleases, T4 DNA ligase, Antarctic phosphatase, Polynucleotide kinase, specific buffers and BSA used for DNA manipulation were purchased from New England Biolabs (NEB; Ipswich, Mass.) and used as recommended by the manufacturer. Polymerases for PCR were exclusively high-fidelity—either Pfu Ultra® II Fusion (Agilent, Santa Clara), Advantage HD Polymerase (Clontech-Takara; Mountainview, Calif.) or Phusion (NEB; Ipswich, Mass.). DNA was extracted from agarose gels using the QIAquick® Gel Extraction Kit (Qiagen; Valencia, Calif.) or the Nucleospin® Gel and PCR Cleanup Kit (Macherey-Nagel Inc; Bethlehem, Pa.) as described by the manufacturer. PCR products were purified using the QIAquick® PCR purification kit (Qiagen; Valencia, Calif.) or the Nucleospin® Gel and PCR Cleanup Kit (Macherey-Nagel Inc; Bethlehem, Pa.) as recommended by the manufacturer. Plasmid DNA was isolated from E. coli transformants that were grown overnight in 4 ml of LB broth with appropriate antibiotic, using a QIAprep® Spin Miniprep kit (Qiagen; Valencia, Calif.) as described by the manufacturer. Gibson assembly was performed using the In-Fusion® Kit (Clontech-Takara; Mountainview, Calif.).

Polymerase Chain Reaction (PCR). Polymerase chain reactions (PCRs) were performed in a T-Gradient thermocycler (Whatman Biometra, Goettingen, Germany). These oligonucleotides were designed from the Staphylococcus aureus NCTC8325 genome (GenBank NC_007795.1) and were purchased from Integrated DNA Technologies (Coralville, Iowa). Oligonucleotides were reconstituted in HPLC grade water (Mallinckrodt Baker; Phillipsburg, N.J.) to 1 mM stock solution. Working solutions of oligonucleotides were prepared by diluting these stock solutions to 10 μM concentration.

Standard PCR mixtures were set up on ice using MQ water or HPLC grade water and prepared as follows: 1×PFU Reaction Buffer, DNA template (2-50 pg plasmid or 50-500 ng genomic template), oligonucleotides at 0.5 μM final concentration, 100 μM dNTPs each (Invitrogen, Carlsbad, Calif.) and 1.25 units of PFU DNA Polymerase (Agilent, Santa Clara Calif.) in a total reaction volume of 50 μl. Initial denaturation was performed at 95° C. for 2 minutes followed by 34 repetitive cycles of the following operations: denaturation at 95° C. for 10 seconds, primer annealing at hybridization temperature (TM−5) for 15 seconds and primer extension at 72° C. for 15 seconds per kb of extension. A final extension at 72° C. for 5 minutes was performed after the final cycle and the reactions were chilled to 4° C.

Agarose gels. Agarose gels were prepared by dissolving agarose in 1×TAE buffer at 100° C. (Fisher Scientific, Pittsburgh, Pa.). Ethidium bromide was added to 0.1 μg/ml prior to pouring the gel. Generally, PCR products were resolved on 1% agarose gels, plasmids and genomic DNA (gDNA) on 0.7% agarose gels. 5×DNA loading dye (50% glycerol and 0.25% bromophenol blue in TAE) was added to the DNA solution in the ratio of 1:4 to reach 1× dye concentration. DNA was loaded on agarose gels and subjected to electrophoresis at about 5 volts/cm until the bromophenol blue had migrated roughly ¾ of the gel length and then visualized under UV light. DNA size was measured against the Hyperladder® (BioLine, Taunton Mass.) series of DNA MW markers.

Plasmid screening. Plasmids used in this study are listed in Table 2 and were constructed by Gibson assembly (Gibson, 2009) using the In-Fusion® kit from Clontech-Takara (Mountainview, Calif.). PCR products and linearized vectors were gel purified (Nucleospin Kit, Macherey Nagel; Bethlehem, Pa.) and assembled using an In-Fusion® Kit (Clontech). Assembled plasmids were introduced initially into E. coli Stellar® Competent cells (Clontech) and verified by sequence analysis (MWG Biotech). Protein expression was carried out in E. coli BL21 (DE3) RIL (Invitrogen) or S. aureus SA178RI.

TABLE 2
Plasmids and Bacterial Strains.

| Plasmid | Description | Reference |
|---|---|---|
| pBADmycHisA | E. coli expression vector with arabinose-inducible promoter | Invitrogen |
| | P_{BAD}; produces fusion proteins with C-terminal Myc and His₆ | |
| | tags | |
| pET21a | E. coli T7 expression vector | Invitrogen |
| pG164 | S. aureus T7 expression vector | D'Elia et al, 2006 |
| pMAD | E. coli/S. aureus shuttle vector for allelic exchange | Arnaud, 2004 |
| pRSFduet | E. coli T7 expression vector with RSF1030 replicon, | Novagen |
| | compatible with pBADmycHisA | |
| pRW | pET21a derivative; N-terminal 6xHis SUMO tag added | Darrell Peterson |
| pT104 | P_{Ars} arsenite-inducible expression in S. aureus | Liu, 2004 |
| pTB | pET21a derivative; Added N-terminal 6xHis tag and TEV | Darrell Peterson |
| | cleavage site | |
| pEW5 | pBADmycHisA derivative; S. aureus L27 MycHis₆ | This work |
| pEW7 | pG164 derivative; S. aureus L27 MycHis₆ | This work |
| pEW15 | pRSFduet derivative; S. aureus Prp | This work |
| pEW21 | pG164 derivative; S. aureus L27 Q7I MycHis₆ | This work |
| pEW22 | pG164 derivative; S. aureus L27 F9Y MycHis₆ | This work |
| pEW23 | pG164 derivative; S. aureus L27 A10S MycHis₆ | This work |
| pEW25 | pG164 derivative; S. aureus Prp | This work |
| pEW26 | pG164 derivative; Prp C34A | This work |
| pEW27 | pG164 derivative; S. aureus L27 | This work |
| pEW28 | pRSFduet derivative; S. aureus Prp C34A | This work |
| pEW29 | pG164 derivative; pre-cleaved L27 | This work |
| pEW34 | pRW derivative; HisSUMO-ysxB | This work |
| pEW35 | pRSFduet derivative; S. aureus Prp C34S | This work |
| pEW37 | pG164 derivative; L27 F8A-F9A MycHis₆ (un-cleavable) | This work |
| pEW39 | pRW derivative; HisSUMO-ysxBC34A | This work |
| pEW40 | pRW derivative; HisSUMO-ysxBC34S | This work |
| pEW41 | pG164 derivative; L27 F8A-F9A (un-cleavable) | This work |
| pEW43 | pG164 derivative; MLKLNLQFF expression | This work |
| pEW50 | pMAD derivative; allelic exchange vector for deletion of the | This work |
| | L27 N-terminal extension | |
| pEW53 | pTB derivative; HisTEVL27 in EcoRI/XhoI | This work |
| pEW56 | pRSFduet derivative; S. aureus PrpV40P | This work |
| pEW57 | pRSFduet derivative; S. aureus PrpN46A | This work |
| pEW63 | pTB derivative; PrpHis6x in NcoI/XhoI | This work |
| pEW68 | pMAD derivative; allelic exchange vector for deletion of L27 | This work, |
| | using a spectinomycin resistance cassette from pCN55 | Charpentier, 2004 |
| pEW72 | pT104 derivative; L27 expression in S. aureus | This work |
| pEW73 | pT104 derivative; L27 Δ2-9 expression in S. aureus | This work |
| pEW74 | pT104 derivative; L27 FF::AA expression in S. aureus | This work |
| pEW75 | pT104 derivative; L27 Δ2-9 and L27 1-9 peptide expression in | This work |
| | S. aureus | |
| pEW76 | pG164 derivative; pre-cleaved L27 His6 expression in S. aureus | This work |
| Strains | Description | Source |
| Stellar | F−, endA1, supE44, thi-1, recA1, relA1, gprA96, phoA, Φ80d | Clontech |
| Competent E. coli | lacZΔ M15, Δ (lacZYA-argF) U169, Δ (mrr-hsdRMS- | |
| DH5α | mcrBC), ΔmcrA, λ− | |
| E. coli BL21 | E. coli B F-ompThsdS(rB− mB−) dcm+ Tetr E. coli gal λ | Agilent |
| DE3 RIL | (DE3) endA Hte [argU ileY leuW Camr] | |
| S. aureus | NCTC8325 cured of Φ11, Φ12 and Φ13 | Novick, 1967 |
| RN450 | | |
| S. aureus | Restriction defective derivative of RN450 | de Azavedo et al., |
| RN4220 | | 1985 |
| SA178RI | 4220-derived CYL316 containing T7 RNA polymerase, Tet^{R} | D'Elia et al., 2006 |
| | 2 μg/μL | |
| ST256 | SA178RI containing pEW27 which expresses L27 from T7 | This work |
| | promoter in the presence of IPTG. Tet^{R} 2 μg/μL, Chl^{R} 15 μg/μL | |
| ST360 | SA178RI L27::Spec^{R} with complementing pEW27. This strain | This work |
| | is IPTG-dependent. Tet^{R} 2 μg/μL, Chl^{R} 15 μg/μL, Spec^{R} | |
| | 250 μg/μL | |

Preparation of electrocompetent cells. Electrocompetent S. aureus cells were prepared from cultures grown in BHI at 37° C. with shaking at 200 rpm, to ˜2×108 cells/ml. The cells were then chilled in an ice water bath for 15 min to arrest growth and harvested by centrifugation at 12,000×g for 15 min at 4° C. The supernatant was carefully removed and the cell pellet was suspended in 50 ml of sterile, ice-cold water and centrifuged again at 12,000×g for 15 min at 4° C. The cells were washed two more times in 50 ml of sterile, ice-cold water. The cell pellet was suspended in 25 ml of sterile, ice-cold 10% glycerol, then the cells were pelleted at 4,000×g for 15 min at 4° C. and resuspended in 2 ml final volume of 10% glycerol to a final concentration of about 1×1010 cells/ml. Cells were distributed in 65 μl aliquots into sterile 1.5 ml microfuge tubes and stored at −70° C.

Transformation of E. coli Gibson assembly reactions (2.5 μl), or ˜30 ng of purified plasmid DNA, were added to 50 μl of Stellar E. coli chemically competent cells (Clontech) previously thawed on ice. This mixture was transferred to a cold 15 ml conical tube. This tube was immersed in a 42° C. water bath for 45 seconds. 1 ml of SOC media (2% (wt/vol) tryptone, 0.5% (wt/vol) yeast extract, 85.5 mM NaCl, 2.5 mM KCl, 10 mM MgCl2, 20 mM glucose) warmed to 37° C. was immediately added to the tube and cells were gently but quickly resuspended by flicking the tube. The cells were incubated for one hour at 37° C. with shaking. Following this recovery period the cells were plated on LB plates with appropriate antibiotic selection.

Transformation of S. aureus. Purified plasmid DNA (5 μl) was added to 65 μl aliquots of electrocompetent S. aureus and incubated on ice for 20 minutes. The mixture was then transferred to an electroporation cuvette with a gap length of 0.1 cm and pulsed one time using the MicroPulser (Bio-Rad, Hercules, Calif.) pre-set S. aureus setting Sta (1.8 kV, 2.5 msec, 25 μF). Following electroporation, one ml of Brain Heart Infusion (BHI) broth (Remel, Lenexa, Kans.) was added immediately to the cuvette and this mixture was then transferred to a 15 ml conical tube. The cells were incubated for 1.5-2 hours with shaking at 30° C. Aliquots (5 μl, 100 μl and the rest of the cells pelleted to a volume not greater than 100 μl) were then spread on TSA plates supplemented with appropriate antibiotics and incubated at 30° C. for 48 hours.

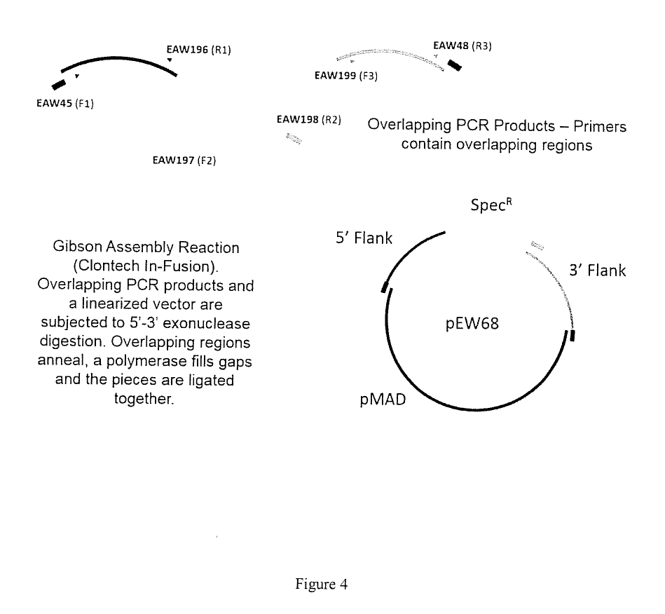

Mutant Creation and Allelic Exchange. The strategy used for creating mutants can be divided into the following steps: (1) The mutant allele was created using Gibson assembly of overlapping pieces of DNA that contained the desired nucleotide changes in the manipulable 15 base pair (bp) overlap regions. Gibson assembly requires that at least 15 bp of any piece of DNA to be assembled to overlap with (be identical to) the neighboring piece to be joined (see FIG. 4). These 15 bp can be included on the primer, and thus can be of any sequence desired. For example, if a point mutation was desired, the codon change could be incorporated in this 15 bp region at the 5′ end of the primer with no concern for how it would affect primer annealing. If a deletion or insertion was required, pieces of noncontiugous sequence could be knitted together in this manner with no intervening or unwanted sequence in between. This provides a mutant allele that can be exchanged using the shuttle plasmid pMAD. (2) Isolation of plasmid from E. coli for sequencing and then transforming the appropriate S. aureus strain and finally, if appropriate, (3) allelic exchange.

Allelic exchange was carried out using the methods described by Arnaud (Arnaud, 2004) with some modifications. See Allelic Exchange in Protocols. A shuttle vector (a vector that can replicate in E. coli and in S. aureus, thereby simplifying the process of plasmid isolation and cloning) called pMAD contains a temperature-sensitive S. aureus origin of replication; it can only replicate independently in S. aureus at temperatures of around 30-32° C. The pMAD vector also encodes an erythromycin resistance cassette for selection and beta galactosidase to allow blue-white screening. This allows us to select for bacteria containing the plasmid using an erythromycin resistance cassette, but then raise the temperature of growth, selecting against extrachromosomal plasmid maintenance. Cells cannot lose the plasmid and continue to live, so this selects for cells in which the plasmid has integrated into the bacterial chromosome. This cointegrate, when plated in the presence of 200 μg/ml 5-bromo-4-chloro-3-indolyl-β-D-galactopyranoside (Xgal), will produce a blue colony. If designed correctly, the homologous sequence on either side of the mutation that we desire will allow this process to occur with efficiency. We then choose blue colonies, culture without selection for plasmid maintenance (in this case, erythromycin) and grow the culture at a permissive temperature for the plasmid. This should allow the plasmid to excise from the chromosome via homologous recombination, hopefully leaving behind the desired mutation. Then we select against plasmid maintenance by raising the temperature. This should allow curing of the plasmid after successive rounds of growth with no erythromycin. To ensure that the plasmid has been cured, white candidate colonies are patched onto erythromycin and must show sensitivity to be considered a possible allelic exchange candidate.

Colony PCR was performed on candidates that had lost the plasmid. For colony PCR, candidates are struck out to single colonies, and a medium-sized colony can serve as the template for PCR. The colony was rubbed onto the bottom interior of a PCR tube. The tube was microwaved the tube on high for one minute, to rupture and dry out the cells, releasing DNA. After microwaving, 10 μl of MQ or HPLC water was added to the cells and they were vortexed, and then centrifuged to remove large chunks of cellular debris which can inhibit PCR. One μl of the supernatant served as template for one 50 μl PCR.

A note on the PCR and sequencing of allelic exchange candidates—care was always taken to amplify the region of interest with primers far outside the boundaries of the regions where homologous recombination with the pMAD derivative could have occurred, and then to sequence with primers only slightly interior to the amplifying ones (still outside the boundaries of possible recombination). This check confirms that there was no accidental incorporation of vector sequence into the chromosome due to a faulty recombination event.

Around the Horn mutagenesis. In lieu of the In-Fusion® reaction, point mutations were sometimes introduced to a plasmid using divergent primers containing the desired change. The primers were first be phosphorylated using Polynucleotide Kinase (PNK) so that they can later undergo ligation. The entire plasmid was amplified by PCR using these primers, then the template vector was digested by addition of 20 units Dpn1 to the reaction. DNA was purified by gel purification or PCR purification, depending on whether non-specific amplification products were present. The linearized product was re-circularized via traditional ligation and introduced into competent E. coli cells by electroporation. Plasmids pEW21, 22 and 23 were created in this manner during the hunt for an un-cleavable point mutant of S. aureus L27.

Expression of proteins in E. coli. Expression of proteins in E. coli strain BL21 DE3 RIL (Invitrogen) was accomplished by growth until OD 0.6 or Klett 90 at 37° C. and then induction with 1 mM IPTG at 30-32° C. The cells were harvested 4 hours post induction by centrifugation at 4000 rpm for 10 minutes and frozen at −80° C. until needed for protein purification. The method of lysis depended on the volume of cells used. For protein purification from a 3 liter culture, cells were resuspended in nickel-NTA column buffer (300 mM NaCl, 25 mM Tris pH 8, 10 mM imidazole) and were then lysed by passage through an EmulsiFlex C3 high-pressure homogenizer (Avestin, Inc., Ottawa). If the cell pellet came from a 400 ml culture or smaller, the cells were pelleted, resuspended in Ni-NTA column buffer, often with EDTA-free protease inhibitor tablets (Roche, Nutley N.J.) and then lysed via mini bead-beater. Samples were centrifuged at high speed (greater than or equal to 12000 rpm) to separate lysate from pellet. The lysate was collected and used for purification, though a pellet sample would be saved for analysis.

Expression of proteins in S. aureus. For protein purification, overnight cultures of respective S. aureus strains containing pG164 derivatives were added to fresh media to a density of around Klett 10. The cells were allowed to grow till mid-late exponential phase (Klett 90). 1 mM IPTG was added and the cells were shifted to 30 C for four hours post induction. Cells were pelleted by centrifuging at 4000 rpm for 10 minutes and resuspended in PBS buffer with EDTA-free protease inhibitor (Roche, Nutley N.J.). Cells were then mechanically disrupted using a mini bead beater and cell lysate was treated as above.

For growth curve analysis of mutant over-expression strains, overnight cultures were inoculated directly into media containing inducer and appropriate antibiotics (TSB with 2 μg/ml tetracycline, 30 μg/ml chloramphenicol and 1 mM IPTG) and monitored at OD600 for 16 hours in a 96 well plate format Biotek Microplate reader.

Growth curves for the complementation strains were performed differently, using a Klett spectrophotometer instead of a microplate reader. These strains contain wild type L27 under an IPTG-inducible T7 promoter. Overnight cultures grown in BHI tetracycline (Tet) 2 μg/ml, chloramphenicol (Chl) 15 μg/ml, spectinomycin (Spec) 250 μg/ml and 1 mM IPTG were inoculated to Klett=3 in fresh media and allowed to grow until the beginning of exponential phase, Klett=30. Five hundred microliter aliquots of those cultures were subsequently harvested at 6000 rpm for 2 minutes in a microcentrifuge. These cells were washed twice with 1 ml BHI (free of IPTG) in order to deplete them of their initial inducer. In the case of the L27 depletion experiment, cells were then inoculated into BHI Tet 2 μg/ml, Chl 15 μg/ml, Spec 250 μg/ml and allowed to continue growing, with Klett measurements taken every hour for 10 hours. In the case where expression was switched to an arsenite-inducible copy of L27, these cells were inoculated into BHI Tet 2 μg/ml, Chl 15 μg/ml, Spec 250 μg/ml, NaAsO2 5 μM.

Polyacrylamide Gel Electrophoresis. Protein samples were analyzed using the Criterion XT protein system (Biorad, Hercules, Calif.) and were separated on either pre-cast Tris-Tricine 16.5% gels or 4-20% gradient gels (Biorad Hercules, Calif.). 2× Tricine loading buffer (for Tricine gels) or 4×XT loading buffer (for glycine gels) was added to samples to achieve a 1× concentration and these samples were then heated for 10 minutes at 75° C. prior to gel loading. The proteins were separated under constant voltage at 200V in 1× Tris-Tricine running buffer (Bio-Rad, Hercules, Calif.). A Precision Plus Dual Xtra Protein Standard dual color marker (2 kDa-150 kDa) (Bio-Rad, Hercules, Calif.) was run with samples to estimate the molecular weight of the protein. After electrophoresis, the gels were rinsed in deionized water three times for 5 minutes each and then stained with Bio-Safe™ Coomassie (Bio-Rad, Hercules, Calif.) followed by destaining in 40% methanol, 10% acetic acid until the image was clear. The bands were visualized under visible light and images were taken using a Fotodyne imaging system (Hartland, Wis.).

Western blotting. Protein samples were loaded onto 16.5% Tris-Tricine precast gels (Biorad) for SDS PAGE and run until the 10 kDa ladder band of the Precision Plus Protein Prestained Standards (Biorad) approached the bottom centimeter of the gel. For detection of L27, gels were electroblotted to a 0.45 micron PVDF membrane for 3 hours at 300 mA in pH 8.3 Tris-Glycine transfer buffer containing 0.05% SDS, to ensure movement of the small, positively charged L27 protein. For detection of the small, acidic Prp protein, gels were electroblotted in Towbin buffer (Tris-Glycine pH 8.3, 20% methanol) for 1 hour 45 minutes at 200 mA. The membranes were blocked with phosphate-buffered saline/tween-20 (PBST) containing 5% skim milk for one hour at room temperature and then rocked overnight at 4° C. with a 1:5000 dilution of primary antibody. L27Myc-His6 was detected with monoclonal mouse anti-Myc antibody (gift from Dr. William Barton) while untagged L27 and Prp were detected using rabbit sera with custom polyclonal antibodies raised to each C-terminally His6-tagged protein in its denatured form (New England Peptide; Gardner, Mass.; Thermo Fisher; Waltham, Mass. respectively). All antibody dilutions were made in 5% skim milk PBST. The membrane was then washed thoroughly with PBST and probed with a 1:10000 dilution of Protein A conjugated to Horseradish Peroxidase (Invitrogen) in 5% skim milk PBST and protected from light for one hour at room temperature. The membrane was protected from light, washed thoroughly with PBST and then Pierce ECL Western Blotting substrate was added for 5 minutes per manufacturer instructions. The blots were then exposed to x-ray film (Bioexpress; Kaysville, Utah) and developed.

Polysome analysis. Overnight cultures of strains for polysome analysis were inoculated into a flask of media containing inducer and appropriate antibiotics (TSB with 2 μg/ml Tet, 30 μg/ml Chl and 1 mM IPTG) beginning at a 1:100 dilution and grown until they reached a Klett reading of 90 or for 4 hours. As noted above, S. aureus expressing Prp C34A will not grow above Klett 50 for over 8 hours, so this strain was grown at a double volume compared to the others so that an equivalent number of cells could be harvested. Cell pellets from at least 100 ml of culture were resuspended in buffer PA (20 mM Tris pH 7.8, 100 mM NH4Cl, 10 mM MgCl2, 6 mM beta mercaptoethanol), broken by bead beating and then cleared by centrifugation for 10 minutes at 13000 rpm at 4° C. The clarified cell lysates were applied to the top of 10-40% sucrose gradients in buffer PA. The gradients were centrifuged for 17 hours at 19000 rpm on an SW-28 rotor at 4° C. Gradients were analyzed for absorbance at 254 nm using a Biocomp Piston Gradient Fractionator with a BIORAD Econo UV Monitor with a Full Scale of 1.0. Data were recorded using DataQ DI-158-UP data acquisition software, as has been previously reported (O'Farrell, 2012). Areas under each peak were calculated using the trapezoid method.

Edman Degradation. Ni-NTA purified L27 Myc-His6 protein samples were loaded onto a 16.5% Tris-tricine precast gel (Biorad) and run until the 10 kDa band of the Precision Plus Protein™ Prestained Standards (Biorad) approached the bottom of the gel. These proteins were electroblotted to a 0.45 m PVDF membrane for 4 hours at 300 mA in a Criterion™ Tank Blotter (Biorad). The membrane was then stained with Coomassie and the appropriate bands were marked and coded and sent to the Iowa State University Protein Facility for Edman degradation. There the protein samples on the membrane were washed six times with deionized water and loaded onto a 494 Procise® Protein Sequencer/140C Analyzer (Applied Biosystems, Inc).

Mass Spectrometry. Ni-NTA purified L27 Myc-His6 protein fractions were separated on a 16.5% Tris-Tricine precast gel (Biorad) and stained with Coomassie Blue. The co-purifying protein was excised from the gel and the gel band was subjected to trypsin digestion and LC-MS analysis of tryptic peptides using a Thermo Electron hybrid LTQ-Orbitrap mass spectrometer.

Pooled sucrose gradient fractions containing the 50S ribosomal assembly intermediates were concentrated and de-salted prior to trypsin digestion and LC-MS/MS. The solutions were diluted with 50 μL 100 mM ammonium bicarbonate. The samples were reduced with 5 μL of 10 mM dithiothreitol in 0.1 M ammonium bicarbonate at room temperature for 0.5 h. Then they were alkylated with 5 μL 50 mM iodoacetamide in 0.1 M ammonium bicarbonate at room temperature for 0.5 h. The samples were digested with 1 μg trypsin overnight and then quenched with 5% (v:v) glacial acetic acid. 3 μL of the final solutions were injected for analysis.

The LC-MS system consisted of a Thermo Electron hybrid LTQ-Orbitrap mass spectrometer system with a nanospray ion source interfaced to a Waters nanoAcquity UPLC system equipped with a Waters NanoAcquity C18 column. 5 μL of the extract was injected and the peptides eluted from the column by an acetonitrile/0.1 M acetic acid gradient at a flow rate of 0.4 μL/min over 60 minutes. The nanospray ion source was operated at 3.5 kV. The digest was analyzed using the double play capability of the instrument acquiring full scan mass spectra to determine peptide molecular weights and product ion spectra to determine amino acid sequence in sequential scans. This mode of analysis produces approximately 10000 CAD spectra of ions ranging in abundance over several orders of magnitude. Not all CAD spectra are derived from peptides.

The data were analyzed by database searching using the Mascot search algorithm against NCBI's non-redundant database. The relative amounts of selected ribosomal proteins in each sample were determined from the MS 1 spectra by measuring the areas under the peaks of individual peptides from each protein and normalizing within each sample to the peptides from the known early assembly proteins L3 and L4.

Development of Prp Assay In order to study Prp enzymology in vitro, an assay was designed using a quenched fluorescent peptide substrate derived from the cleavage motif on L27. The peptide KLNLQFFASKK (GI:15927225; SEQ ID NO:1) was chosen for its content of hydrophilic residues and fluorophore 2-amino benzoic acid (2-abz) was conjugated to the N terminus, with a dinitrophenol (Dnp) quencher conjugated to the C-terminus. The resulting peptide substrate (2abz-KLNLQFFASKK-Dnp; SEQ ID NO:1) was ordered from United Peptide in Herndon, Va. (now United Biosystems). The substrate was initially resuspended to 2 mM with a buffer containing 25 mM Tris pH8 and 300 mM NaCl. Fluorescence was measured using an excitation wavelength of 325 nm and an emission wavelength of 425 nm using a Photon Technology International QuantaMaster steady-state fluorescent spectrometer. Prp was initially purified and analyzed as a His6-SUMO N-terminal fusion protein from plasmid pEW34. His6-SUMO fusions were also made of PrpC34A (pEW39) and PrpC34S (pEW40).

### Results

We previously noted that L27 proteins in S. aureus and other Firmicute bacteria have an N-terminal extension that is not present in E. coli L27 (Spilman, 2012). Cleavage of the extension was predicted to generate an N-terminus identical to that found in E. coli L27, which is known to aid in peptidyl transfer. Our bioinformatic analyses revealed that all sequenced Firmicutes, Fusobacteria, and Synergistetes, as well as some Thermatogae and Tenericutes, encode an L27 containing an N-terminal extension with the conserved cleavage motif. Everything thus far known about L27 suggested that this N-terminal extension would be highly toxic to the ribosome because it would occlude the conserved residues that stabilize tRNAs at the PTC. Only the post-cleavage form of L27 was found in mature B. subtilis ribosomes, supporting the validity of this theory (Lauber, 2009).

Taken together, these data support the existence of a group of evolutionarily related bacteria that exhibit a fundamental difference in the basic biology of the ribosome, involving a previously undescribed ribosomal processing event. Since L27 is positioned at the heart of the ribosomal active site, where it plays a key role in translation (Maguire, 2005), we sought to elucidate further the role of this N-terminal extension in S. aureus biology.

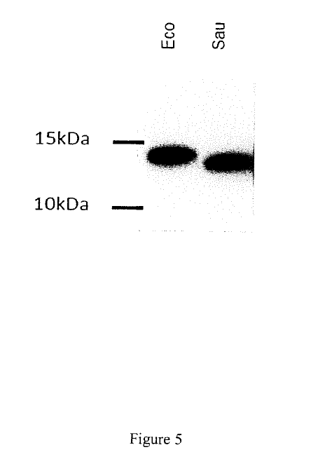

We tested the predicted N-terminal cleavage of S. aureus ribosomal protein L27 by comparing the products of the cloned full length gene in S. aureus and E. coli, as we had previously done for the phage 80α capsid and scaffold proteins (Spilman, 2012). To identify and purify S. aureus L27 in E. coli, we introduced C-terminal Myc and His6 tags using vector pBADMycHis A (Invitrogen). The same tagged L27 cassette was introduced into the T7 expression plasmid pG164 (D'Elia, 2006) for expression in S. aureus. The His-tagged proteins in clarified lysates from each overexpression strain were batch adsorbed to Nickel NTA resin (Clontech) and purified proteins were then examined on Western blots probed with anti-Myc antibodies. L27 isolated from S. aureus is approximately 1 kDa smaller than the same protein isolated from E. coli (FIG. 5), consistent with the predicted N-terminal cleavage by a S. aureus protease that is absent from E. coli. The L27 protein expressed in S. aureus was extracted from the gel and subjected to N-terminal protein sequencing by Edman degradation. The first four residues were ASKK, confirming the predicted processing of L27 in S. aureus at the conserved cleavage motif. Apart from the common removal of the N-terminal formyl methionine, this is the first example of a specific N-terminal cleavage of a bacterial ribosomal protein precursor.

A protease or peptidase is an enzyme that can catalyze cleavage of a protein or peptide substrate. There are now seven classes of proteases, the most well-studied of which are the hydrolases. Cysteine, serine and threonine proteases use their respective side-chain nucleophiles to attack the carbonyl carbon of the substrate peptide bond which then relinquishes its link to the amide nitrogen and forms a covalent enzyme/substrate acyl intermediate. This intermediate is hydrolyzed and the product is released. In contrast, aspartate, glutamate and metalloproteases perform acid/base catalysis using an activated water molecule. The seventh and newest group of proteases are not hydrolases—asparagine amidine lyases self-cleave using a coordinated water molecule to form a succinimide intermediate that cleaves the Asn-Asn peptide bond. Intein auto-cleavage domains utilize this mechanism (Rawlings, 2011).

From sequence comparison of S. aureus L27 with phage 80α capsid and scaffold as well as expression of those proteins in E. coli, it seemed extremely unlikely that the specific N-terminal cleavage event was autocatalytic. That left one of six possible hydrolases, all of which have some identifiable set of catalytic residues that would have to be completely conserved across mechanistic homologs. There have been many classified proteases—the MEROPS database has an extensive collection of annotated and experimentally confirmed protease families.

We expected the protease responsible for S. aureus L27 cleavage to be essential and conserved among bacteria with the L27 N-terminal cleavage motif. Our attention was drawn to an open reading frame of unknown function, first designated ysxB in B. subtilis (locus tag SAOUHSC_01756 in S. aureus NCTC8325), located between the genes encoding L21 (rplU) and L27 (rpmA). The occurrence of this intervening reading frame was first noticed in a sequencing project in 1985 which showed both that by sequence, L27 in B. subtilis was N-terminally extended compared to that in E. coli, and that there was a gene upstream of L27 that bore no homology to L21 (Ferrari, 1985). It was also noted in a bioinformatic study of horizontal gene transfer between Gram positive bacteria that the intervening reading frame belonged to an established conserved domain family named COG2868 and was maintained between L21 and L27 among Bacilli, Spirochaetales and Theromotogales. (Garcia-Vallve, 2002).

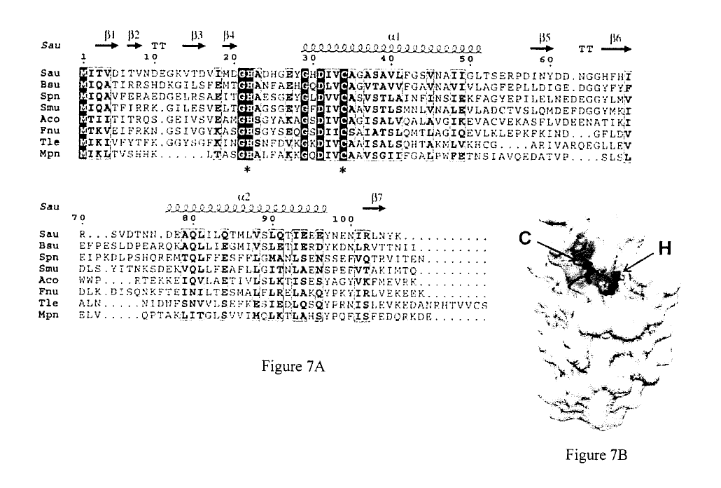

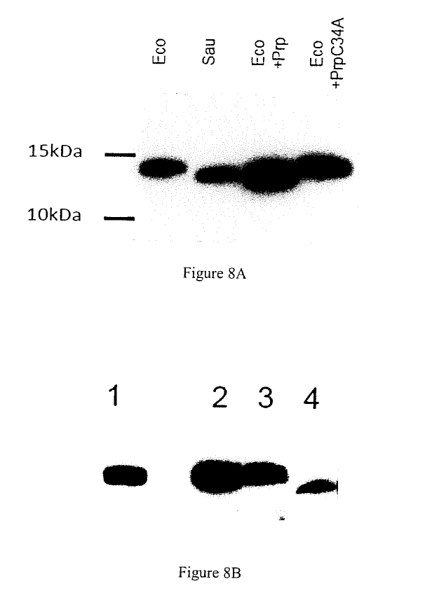

YsxB was an attractive candidate for the protease for several reasons: First, all bacteria that encode an L27 with the N-terminal extension also carry a ysxB gene (FIG. 8). Second, both rpmA and ysxB were classified as essential in S. aureus by saturation transposon mutagenesis (Chaudhuri, 2009) and in an earlier antisense RNA study (Ji, 2001). Third, the YsxB protein has features characteristic of a protease. The YsxB structure was solved in a structural survey of S. aureus proteins (PDB ID: 2P92) and was grouped with other similar structurally characterized proteins containing a common domain in NCBI's Conserved Domain Database (CDD) under the designation DUF464. The crystal structure of a DUF464 member from Thermatoga maritima (1S1L) was published in 2005, and the authors noted that the conserved residues could possibly constitute a catalytic center (Shin, 2005). The structures of two additional family members from Streptococcus mutans (PDB ID: 2GOI) and Streptococcus pneumoniae (PDB ID: 2IDL) have also been solved. DUF464 proteins are distinguished by a pair of invariant histidine and cysteine residues with conserved spacing that form the classic catalytic dyad of a cysteine protease (FIG. 7A), and are clustered together in a cleft on observed DUF464 structures (FIG. 7B). In addition to the ysxB homologues present upstream of rpmA, there are a number of phages that encode a DUF464 family member. One member of this family has been shown to be a bona fide bacteriophage prohead protease, responsible for capsid protein cleavage in streptococcal phage Cp-1 (Martin, 1998).

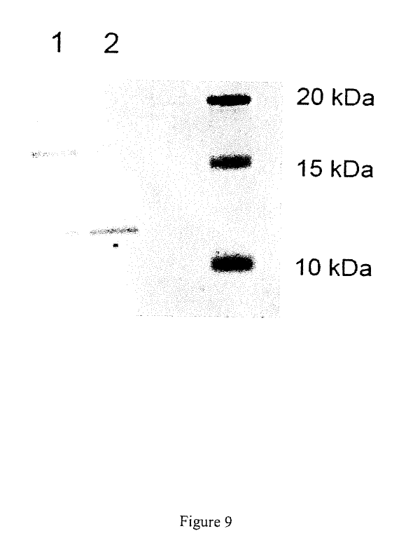

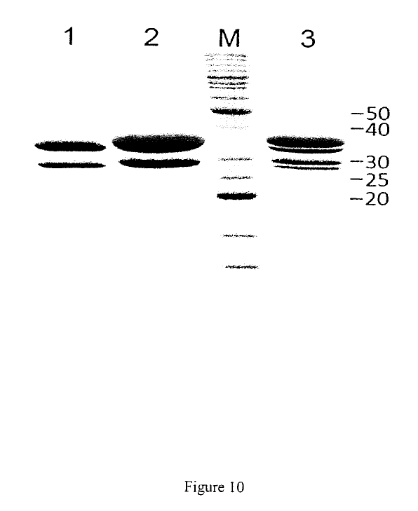

When S. aureus L27 Myc-His6 and untagged YsxB were co-expressed in E. coli, the resulting tagged and purified protein was the same size as L27 produced in S. aureus; both are approximately 1 kDa smaller than L27 expressed in E. coli in the absence of YsxB (FIG. 10). Edman degradation confirmed that the N-terminus of S. aureus L27Myc-His6 co-expressed with YsxB in E. coli was also ASKK (SEQ ID NO:18). These data demonstrate that YsxB can perform specific N-terminal cleavage of S. aureus L27 in E. coli and confirm that this conserved gene of previously unknown function encodes the L27 protease. We have named this gene prp, for phage-related ribosomal protease. Further evidence to support the classification of this protein as a cysteine protease was provided by mutation of the predicted catalytic cysteine residue, C34, to Ala or Ser, which resulted in the inactivation of Prp (FIG. 8A, lane “Eco+PrpC34A”; FIG. 8B lane 2). Furthermore, when the un-cleaved L27Myc-His was purified from a strain co-expressing Prp C34A or C34S, the mutant Prp co-purified as an 11 kDa band on the gel accompanying the 13 kDa L27Myc-His6 protein (FIG. 9). The identity of the 11 kDa co-purifying band was confirmed by mass spectrometry. These observations indicate that the mutant protease remains tightly bound to its substrate in the absence of catalysis. They also show that replacement of cysteine with serine in the catalytic site does not allow Prp to function as a serine protease, despite the presence of a conserved aspartic acid or asparagine in the DUF464 motif.

The original identification of Prp was based on the cleavage of the major capsid and scaffolding proteins of bacteriophage 80α in S. aureus. We therefore confirmed that Prp was able to carry out the previously observed N-terminal cleavage of these two phage structural proteins upon co-expression in E. coli (FIG. 10). The small differences in electrophoretic mobility of these two proteins were shown to result from the expected sequence-specific cleavages by determining the total mass of the processed proteins by MALDI-MS and further confirmed by LC-MS of tryptic peptides. Prp thus serves an additional function in S. aureus, as the prohead protease for 80α and related staphylococcal phages whose capsid and scaffold proteins share the conserved N-terminal cleavage motif.

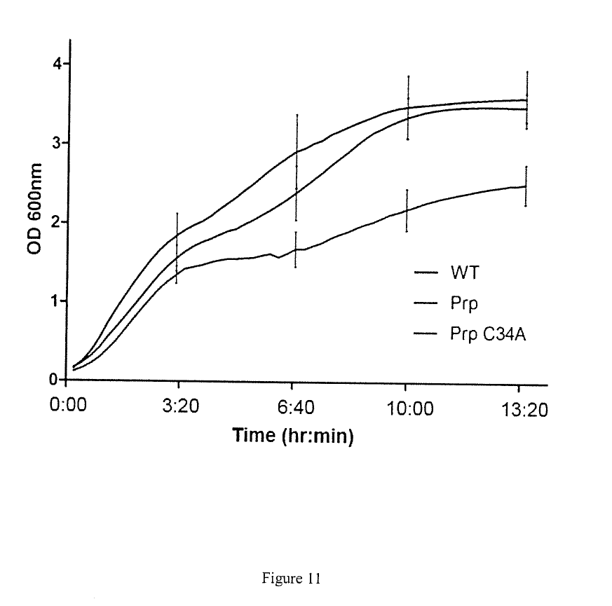

Both L27 and Prp are essential in S. aureus. One common approach is to examine the effects of mutations in essential genes is to over-express mutant allele and assess the resulting phenotypes. We investigated the effects of mutant Prp by examining the growth of strains overexpressing catalytically inactive Prp C34A compared to wild-type Prp, using a S. aureus T7 expression system (D'Elia, 2006). Expression from the plasmids in S. aureus strain SA178RI was induced for 16 hours at 32° C. and growth was compared to the same strain with empty vector. Overproduction of Prp C34A led to a marked growth defect compared to overproduction of the wild-type Prp (FIG. 11). (Note that the expression strain still contains native L27 and Prp.)

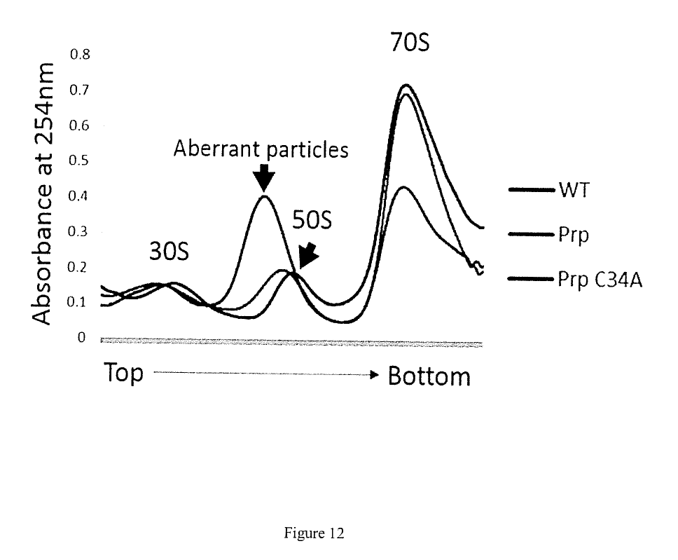

The role of Prp in S. aureus ribosome assembly was investigated by examining ribosomal profiles from cells overexpressing either wild-type Prp or inactive Prp C34A. Overexpression of Prp C34A had a very significant effect on ribosomal subunit profiles, resulting in a 50% decrease in mature 70S ribosomes. There was also an accumulation of what appeared to be pre-50S assembly intermediates (FIG. 12), suggesting that proper ribosome assembly is dependent on L27 cleavage. However, the dominant negative phenotype of Prp C34A could have been a result of catalytically inactive Prp binding to L27 without cleaving, and thereby sequestering it from participation in ribosomal assembly. L27 has a known role in 50S subunit assembly, and the ribosomal subunit pattern in the strain overexpressing Prp C34A resembled what has been observed with an intermediate depletion of L27 from the large subunit (Wower, 1998). The effect of wild-type Prp overexpression on ribosomal subunit proportions did not deviate significantly from empty vector.

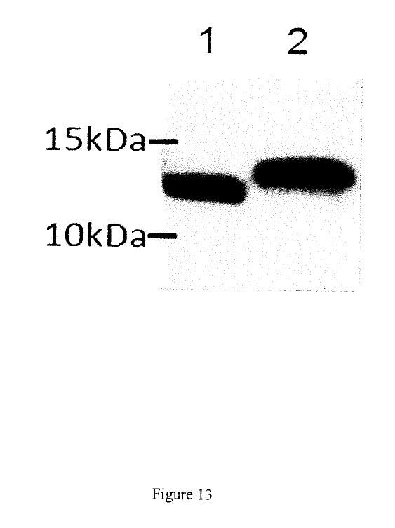

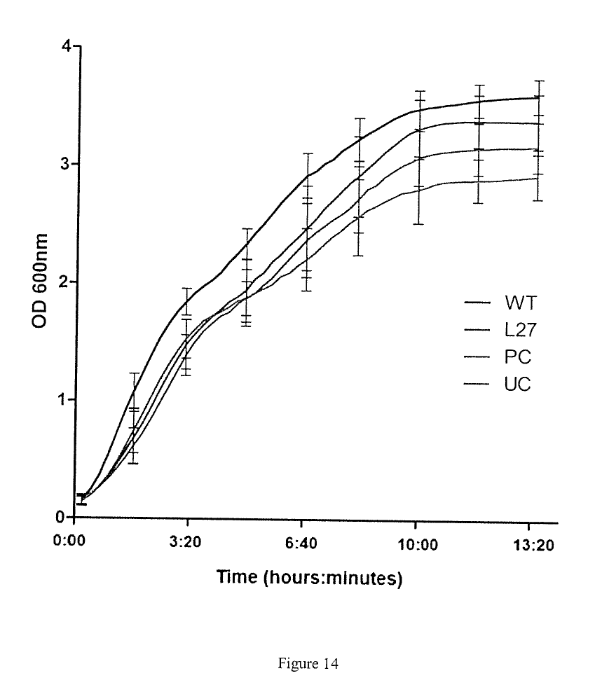

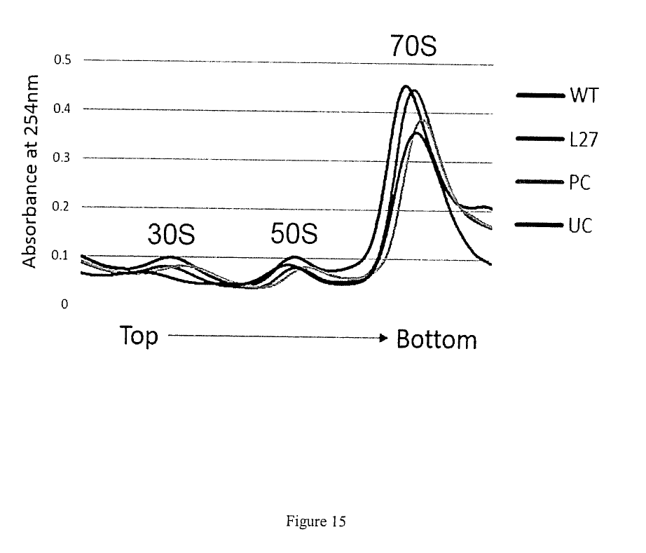

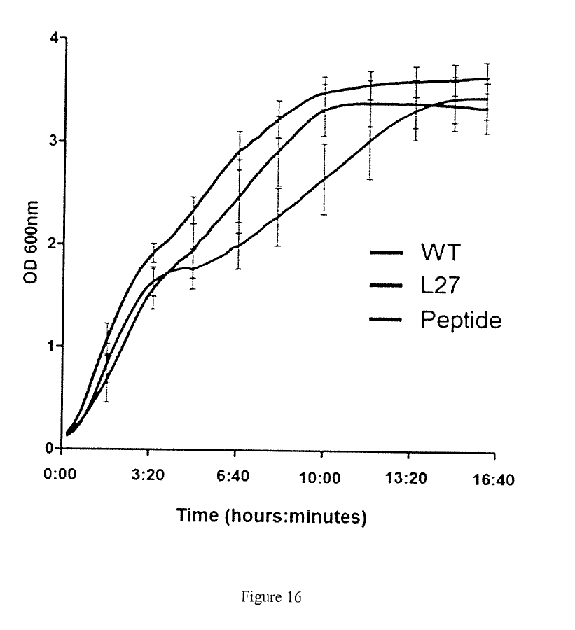

Variant forms of L27 were also overexpressed to determine their effects on cell growth and ribosomal subunit distribution: wild-type, pre-cleaved (deletion of L27 residues 2-9) or un-cleavable (L27 F8A-F9A, giving motif MLKLNLQAAA; SEQ ID NO:34; FIG. 13). In contrast to the strong dominant negative effect of Prp C34A, overexpression of the mutant forms of L27 showed little effect on cell growth (a mild defect was seen with the un-cleavable mutant; FIG. 14) and yielded ribosomal subunit profiles similar to cells expressing empty vector (FIG. 15). The presence of native levels of L27 and Prp in this strain likely masks any effects of these L27 N-terminal cleavage mutants in ribosomal assembly. We also investigated the effect on S. aureus growth of overexpressing the L27 N-terminal peptide that would be released upon cleavage (MLKLNLQFF) (SEQ ID NO:35; GI:15927225). The growth curve was qualitatively and quantitatively different from that of L27 overexpression, exhibiting a pronounced lag in the middle of the exponential growth phase before resuming growth (FIG. 16). Preliminary examination of the ribosomal subunit profile of this strain showed no anomalies. How the overexpressed peptide alone is able to exert this striking effect on S. aureus growth is an open question. It is possible that the cleaved peptide acts as a competitive inhibitor of Prp, blocking L27 cleavage, or that it has another role in the cell, perhaps in a regulatory capacity.

We further characterized the aberrant ribosomal particle produced in the presence of Prp C34A to determine the stage of assembly at which they were trapped. The composition of both wild type and aberrant 50S particles were analyzed by mass spectrometry (LC MS/MS). We chose this approach because sucrose gradient fractions are a complex mixture of both ribosomal proteins and co-migrating cellular protein complexes with a similar sedimentation coefficient. Furthermore, these cells also contain wild-type ribosomes. LC MS/MS of tryptic peptides allowed us to confirm the presence of specific ribosomal proteins in these fractions, while examination of the MS 1 spectra of the peptides from individual ribosomal proteins allowed us to determine the relative amounts of several specific proteins within each sample. Chromatograms for the precursor masses of two tryptic peptides from all large subunit ribosomal proteins detected were generated from both particle types. Peptide peak areas for identical peptides in both samples were calculated using conventional tryptic digest mass spectrometry MS 1 spectra.

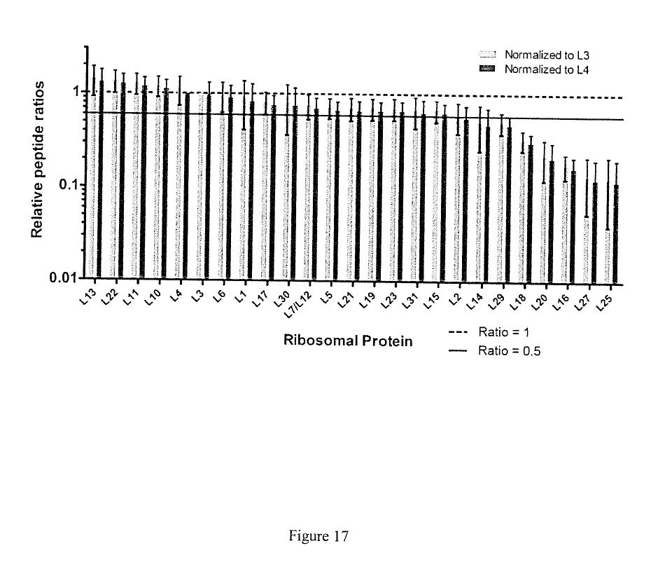

We were able to compare peptide abundances for all detected r-proteins to those of two early assembly proteins, L3 and L4 (FIG. 17). The ratio of L3 and L4 to each other and to the vast majority of other ribosomal proteins detected remained the same, within a standard deviation above and below, in both the 50S and aberrant particles, consistent with the inherent stoichiometry of ribosomal proteins. However, the internal ratios of later assembly proteins like L16 and L25 to early assembly proteins L3 and L4 were noticeably diminished in the aberrant particles. The standard by which a particular protein was judged as sub-stoichiometric with respect to L3 or L4 was a mean ratio to those proteins of 0.5 or lower. The sub-stoichiometric proteins, from greatest amount detected to least, were L2, L14, L29, L18, L20, L16, L27 and L25. Not noted in FIG. 17 are proteins for which only one tryptic peptide was measured: L24, L32, L33 (sometimes referred to as L33a) and L36. We did not detect L9, L28, L34 and L35.

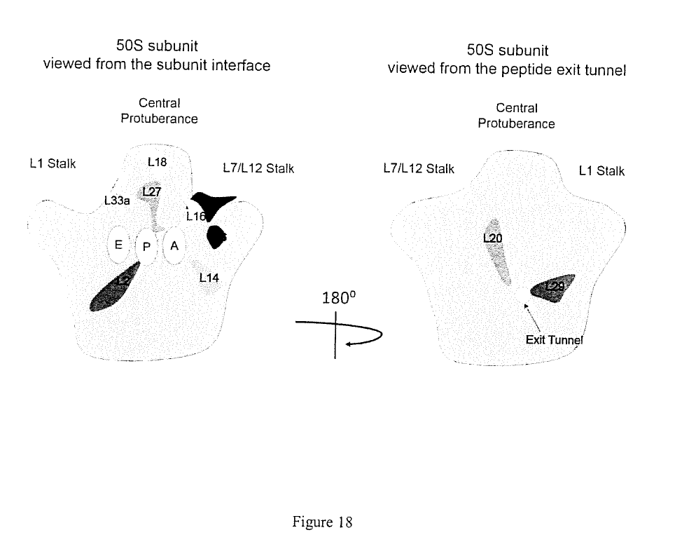

The loss of L16, L27 and L25 is consistent with a ribosome trapped a late stage of assembly. R-proteins L16, L18, L25, L27, L33, and L36 are all situated near the PTC and the central protuberance (see FIG. 18), which are thought to be the final regions assembled (Chen, 2013; Jomaa, 2013; Li, 2013). There were losses of some unexpected r-proteins, however. Ribosomal protein L20 is not considered a late assembly protein in the current assembly maps, and is situated in the interior of the central body of the ribosome. Its decrease is consistent with data previously generated on E. coli ribosomes assembled without L27 (Wower, 1998). However, the E. coli aberrant 50S subunits formed without L27 present in that study also failed to contain L21, which was not significantly absent from our aberrant 50S particles. L2 and L14 are situated near the interior of the ribosome and far away from the catalytic site. L20 is extremely interior to the ribosome and L29 is situated over the exit tunnel, one of the first regions thought to be assembled (FIG. 18).

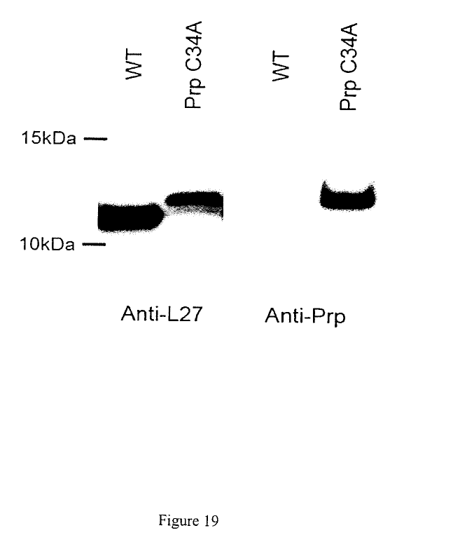

These particles are most likely incomplete assemblies of the 50S subunit. Ribosomal proteins L9, L28, L34 and L35 were not detected in either sample. However, there are legitimate precision concerns with this type of analysis for ribosomal peptides. Conventional tryptic digest LC MS/MS is particularly problematic for small, highly negatively or positively charged proteins due to their distribution of tryptic residues. Only one peptide was found from each L24, L32, L33 and L36, so they are not included in FIG. 17. There are very few predicted detectable tryptic peptides from ribosomal proteins L28, L34, L35 and the small negative protease Prp. Prp was detected in neither sample by mass spectrometry, but was detected in aberrant particles by immunoblot (see below and FIG. 19). There is only one measurable tryptic peptide produced in Prp predicted digestion—even in the mass spectrometry performed on the strong co-purified PrpC34A band (FIG. 9) Prp spectra were only detected in 3 scans, though of the proteins detected (mostly keratin contamination) it represented the only protein that could produce a band of that molecular weight.

Rabbit polyclonal antibodies were raised to both L27 and Prp to allow immunodetection by Western blot. L27 was detected in aberrant particles as an imbalanced doublet that appears to represent large amounts of un-cleaved L27 and only minor amounts of cleaved L27, while wild type particles only contained the smaller cleaved L27 band. In addition, Prp was present in the aberrant particles and absent in normal 50S subunits (FIG. 19). It seems that the bound complex of un-cleaved L27 and PrpC34A was assembled into many of the ribosomes in the strain overexpressing PrpC34A, which would explain their arrest in development.

The phenotype displayed when mutant Prp is expressed was consistent with a role for Prp and L27 cleavage in ribosome assembly. Interpretation of those results is complicated, however, by the presence of wild type Prp and L27. In order to more accurately assess the roles for Prp and L27 cleavage, it was necessary to be able to examine the effects of L27 mutations directly. For this it was necessary to create a complementation system that would allow us to toggle between wild-type L27 and one of our mutants of interest. Further, we sought to determine whether separate expression of the post-cleavage peptide would have an effect.

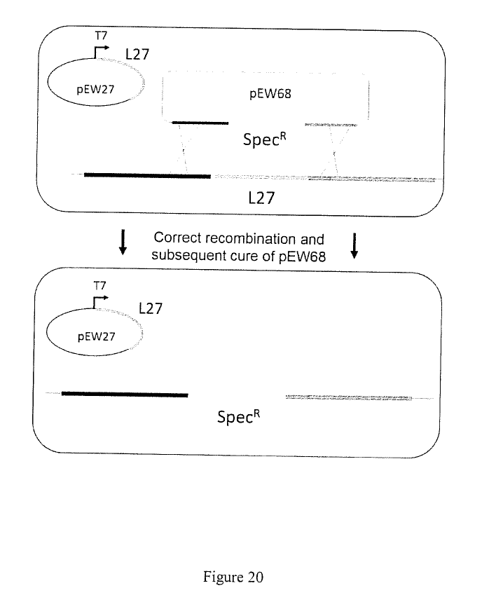

Creation of a complementation system in S. aureus required a fairly complicated strain construction regime. Strain SA178RI carries a chromosomal copy of the gene for T7 RNA polymerase under an IPTG-inducible promoter. This strain was transformed with pEW27, vector that expresses Sau L27 from a T7 promoter. The resulting strain ST256, was subjected to allelic exchange to knock out the chromosomal copy of L27 using plasmid pEW68. A spectinomycin resistance cassette was substituted for chromosomal L27 while maintaining the cells in the presence of IPTG, allowing pEW27 to complement via ectopic expression of L27. All candidates that had resolved pEW68 were also spectinomycin resistant and IPTG dependent. IPTG dependence indicated that L27 must be expressed from pEW27 to support growth. This strain thus represents the first conclusive genetic evidence that L27 is essential in S. aureus. This new L27 complementation/deletion strain was named ST360 (FIG. 20).

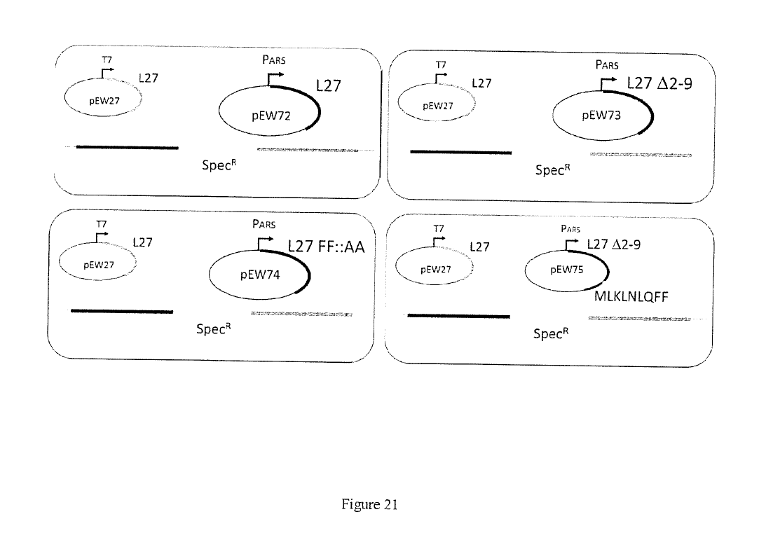

A compatible plasmid containing a kanamycin resistance cassette and an arsenite-inducible promoter, pT104 (Liu, 2004), was introduced to ST360 as the vector for mutant L27 expression. We first performed a control experiment to show that the presence of arsenite alone will induce enough L27 from that promoter to allow growth. Plasmid pEW72 is a pT104 derivative expressing wild type Sau L27, and it is able to complement the L27 chromosomal deletion in the presence of 5 μM NaAsO4. There is no leaky expression from the promoter—in the absence of IPTG and arsenite, there is no growth of that strain. The pT104 backbone was used to construct plasmids pEW73-75, which expressed variant forms of L27 to test their ability to complement an L27 deletion (FIG. 21).

Plasmid pEW73 expresses “pre-cleaved” L27 (Δ 2-9) that is missing the N-terminal cleavage motif, making it effectively equal to the “short” Eco L27. We used Edman degradation on purified pre-cleaved L27 with a C-terminal His6 tag (pEW76) to determine that this protein loses its N-terminal Met when produced in S. aureus, precluding any obfuscation of the peptidyl transferase center. It is essentially identical to the studied E. coli or T. thermophilus N-terminus of L27 that has been visualized at the PTC in the ribosomes of those organisms (Polikanov, 2014).

Plasmid pEW74 expresses Sau L27 F8A, F9A, an un-cleavable mutant of Sau L27. We confirmed that this protein with N-terminal residues MLKLNLQAAA was no longer cleaved by Prp via Western blot, as shown in FIG. 13. We also found that although Prp binds tightly to the normal L27 motif, we were not able to pull down native Prp when this tagged un-cleavable protein was expressed and purified from S. aureus. This seems to indicate that Prp can no longer bind the FF::AA mutant, suggesting an important role for the Phe residues in enzyme/substrate binding.

Plasmid pEW75 expresses pre-cleaved L27, and, under the same promoter but using a separate RBS downstream, the post-cleavage released peptide “MLKLNLQFF.” Small peptides have been shown to have a role as hormones in Enterococcus fecaelis, a related bacterium that also encodes long L27, to determine whether the peptide had an effect when separated from its original source or could compensate for pre-cleaved L27.

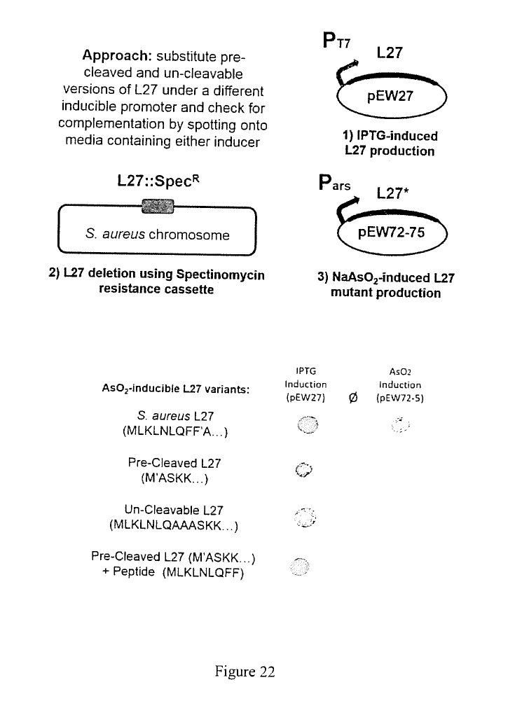

Of these plasmids, only pEW72 expressing wild type L27 could complement an L27 chromosomal deletion in ST360. All strains were spotted onto solid media containing 1 mM IPTG, no inducer, or 5 μM NaAsO2. All grew on IPTG (pEW27-dependent growth), all died without any inducer (no leakage from either promoter) and none but the strain containing pEW72 survived on arsenite (FIG. 22, bottom). This indicates that not only is L27 cleavage essential, it also must be tightly regulated. The peptide may yet have a role in cellular processes, but that role is insufficient to promote S. aureus growth without regulated L27 cleavage.

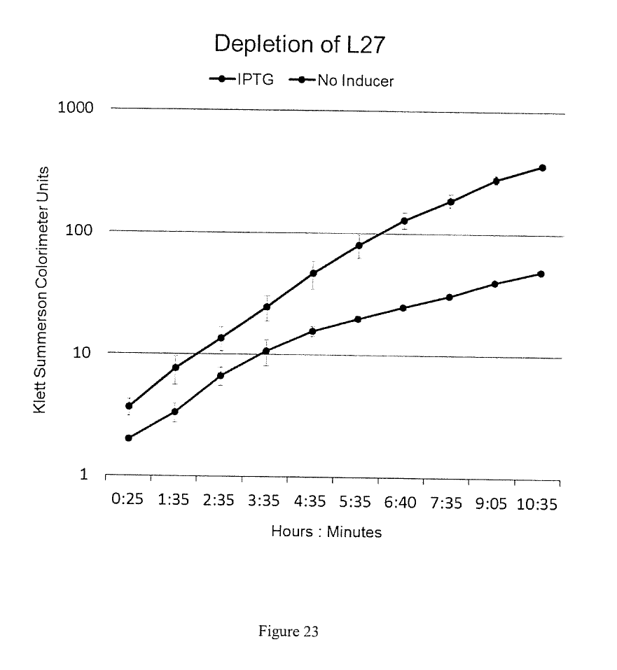

Growth curves were measured for the strains containing only IPTG-inducible L27 and those that also contained an arsenite-inducible alternative wild type L27, pre-cleaved L27 and un-cleavable L27, mentioned previously. The first growth curve (FIG. 23) is a comparison between cells supplied with IPTG to allow L27 production versus those depleted of L27 by washing cells and then inoculating them into media without IPTG. IPTG-induced L27 production allowed cells to complete at least six doublings in under nine hours. L27 depletion caused an arrest in growth after just over four doublings in ten hours. The continued growth during L27 depletion is likely due to the presence of previously assembled, functioning ribosomes in the initial inoculum. The first doubling results in half the residual functional ribosomes, one fourth in the second doubling, one eighth in the third doubling, one sixteenth in the fourth—this seems to be the point at which growth can no longer continue at anywhere near the normal rate.

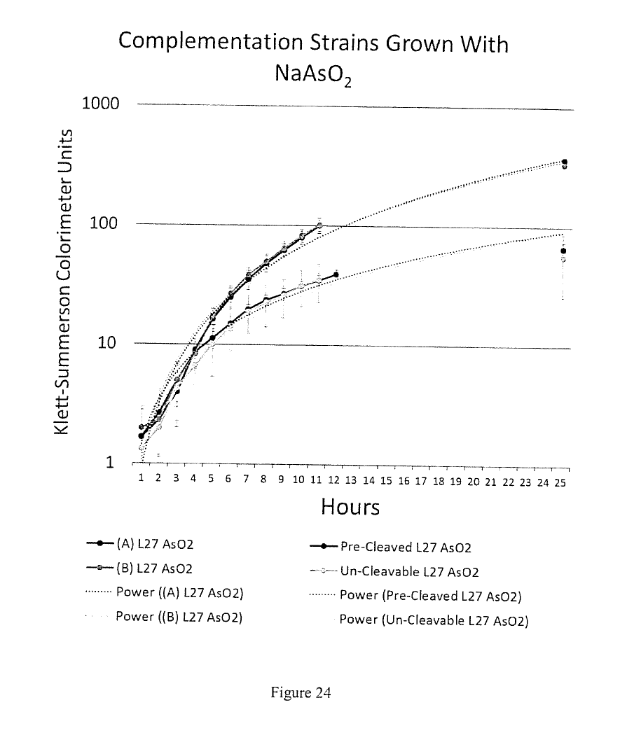

The arsenite-inducible complementation strains were measured after induction of the mutant L27 allele. In FIG. 24, arsenite-induced wild type L27 completed eight doublings in 24 hours, with six doublings occurring in the first ten hours. This is slightly slower than cells supplied IPTG, almost certainly because the T7 promoter under IPTG control produces more L27 than does the arsenite inducible promoter. However, cells supplied with pre-cleaved L27 or un-cleavable L27 from the arsenite promoter managed to double only five times in 24 hours, with the first four doublings taking place in about seven to eight hours, and the fifth doubling taking place around eighteen hours after inoculation. The growth pattern displayed in conditions of L27 deprivation and in the presence of pre-cleaved or un-cleavable L27 expression was nearly identical. These results suggest that supplying pre-cleaved or un-cleavable L27 does not result in the production of new active ribosomes, as is most probably the case with L27 depletion from the cell.

Existing structures of Prp family members demonstrate that Prp forms its own completely new clan of cysteine proteases, dubbed clan CR by the MEROPS database which catalogs and classifies peptidases, their substrates and their inhibitors. Within clan CR, Staphylococcus aureus Prp is now the prototype member of Family C. 108 (cysteine proteases, group number 108). It is as yet unknown how Prp binds its substrate, so loop refinement, docking and molecular dynamics were undertaken to explore possible models for Prp-substrate interaction.

Structural modeling through protein sequence homology has become an extremely important tool in deciphering hitherto unknown functions in conserved domains. This modeling attempt was relatively conservative, only modeling two small loops onto an existing crystal structure, docking a short peptide and then subjecting that modeled complex to a short round of molecular dynamics simulation. The mechanism and necessary requirements known for cysteine proteases informed the modeling attempt. As discussed in Chapter 4, cysteine proteases are hydrolases that use nucleophilic attack of the carbonyl carbon in the scissile bond. This is achieved via a catalytic dyad, His and Cys, or a catalytic triad, His, Cys and Asp or in some cases Asn. There is a residue in the Prp motif that is usually Asp or Asn, three residues prior to the catalytic Cys, but the role of that residue in catalysis is not yet known. For the purpose of this modeling experiment we assumed that Prp relies on the catalytic dyad because those residues are perfectly conserved, while the other residue differs.

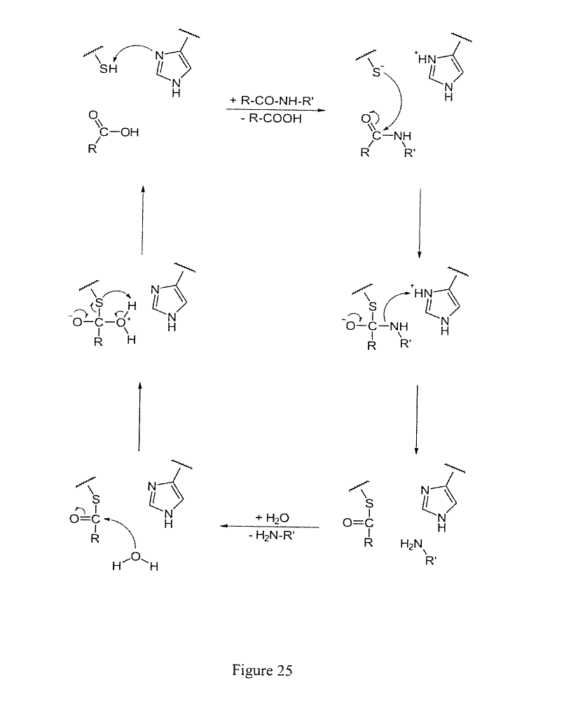

In the mechanism of catalysis for a cysteine protease with a catalytic dyad (FIG. 25), the imidazole group of the His polarizes the sulfhydryl of the Cys and abstracts a proton, producing the ionic pair imidazolium and thiolate. The thiolate ion attacks the carbonyl carbon of the substrate scissile bond, producing a tetrahedral intermediate with an oxyanion. The nitrogen in the tetrahedral intermediate then abstracts a proton from the imidazolium moiety. This breaks the bond between the nitrogen and the enzyme-substrate complex, forming an amino terminus, which allows that peptide chain to dissociate. The carbon-oxygen double bond is restored and a covalent acyl-enzyme intermediate is formed on the catalytic Cys. A water molecule attacks the carbonyl carbon of the thioester bond, which forms another tetrahedral intermediate containing an oxyanion. This intermediate is hydrolyzed and the C-terminus of the substrate is released as a free acid. The formation of an oxyanion during the tetrahedral intermediate stage occurs twice in this mechanism, which typically requires stabilization via a pocket in the enzyme with available amide nitrogens as hydrogen bonding partners.

From this mechanism we know that the sidechains of the catalytic dyad must be in close enough proximity to achieve ionization, and that a required feature of the enzyme would be a region that could stabilize the oxyanion of the tetrahedral intermediate.

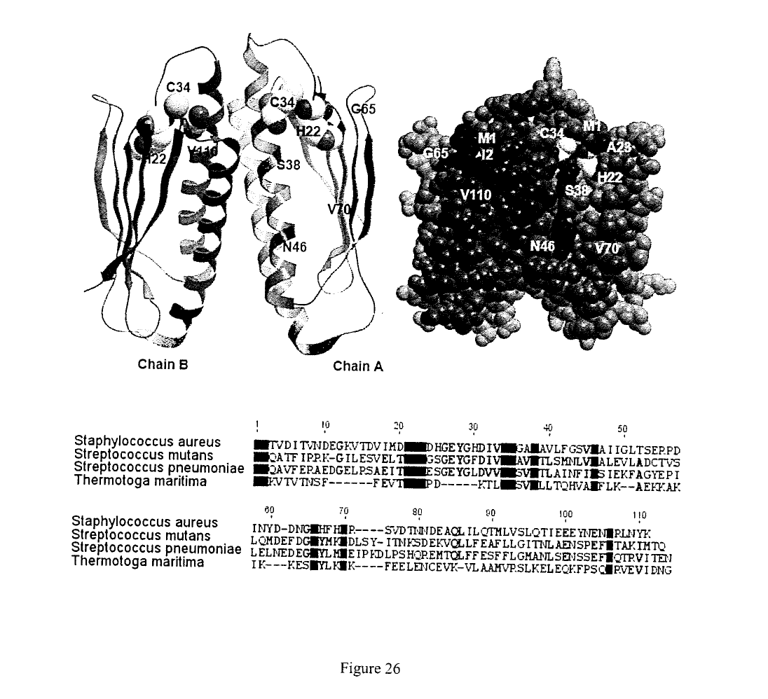

Modeling began by examining existing structures of Prp in the PDB. These include homologs from Thermatoga maritima (1S12), Streptococcus pneumoniae (2IDL), Streptococcus mutans (2G0I) and Staphylococcus aureus (2P92). They all share the unique two layer sandwich dimeric structure of pseudo twofold rotational symmetry. Each monomer is composed of two helices in contact with one side of a five stranded beta-sheet. The fold conservation is remarkable, given average sequence identities of 20-30 percent. FIG. 26 shows conserved residues mapped onto the 2IDL backbone.

This fold was completely novel in 2005 when the T. maritima structure was solved, so much so that even though the authors conjectured that the conserved residues formed a catalytic center, the closest structural homologs they could identify were a hydratase and a gene of unknown function from yeast, leading them to pass on functional speculation (Shin, 2005). The Streptococcus structures were solved by crystallographic consortia and remain unpublished. By the time of writing, the paper about S. aureus 2P92 had just been published, in which the authors speculate that because L21, Prp and L27 exist in an operon, it would make sense for them to all form a complex related to ribosome assembly, with Prp as the acidic “glue” between alkaline L21 and L27. They demonstrated that Prp is a dimer in solution, validating our identical finding (Wall, Johnson, Peterson and Christie, in prep). They did not discern that the protein contained the catalytic center characteristic of a cysteine protease, even though they had literally thousands of sequences at their disposal, and did not discuss the lack of crystallization of a 10 amino acid loop that fell in between the completely conserved His and Cys residues (Chirgadze, 2015).

A striking feature of these structures is their apparent lack of catalytic capacity. The catalytic dyad must be at some point be solvent exposed to accept substrate, and the His and Cys must face each other in such a way that the His can abstract a proton from the sulfhydryl group of the Cys. No structure available had these features. Though the His and Cys is sometimes be in close proximity on the structures, as in 2IDL (FIG. 26) the rotation of their sidechains is influenced by the flexible loop that lies between them, orienting them in opposite directions. This might have been the confounding factor in the failure to identify this protein as a cysteine protease.

The PDB file of the incomplete Staphylococcus aureus Prp/YsxB homolog dimer (2P92) was analyzed in SYBYL-X 2.1, a graphical interface program for molecular modeling. It was immediately apparent that not only was one of the catalytic residues (His 22) missing from an active site, but also that the inward-facing side chain rotamer of active site Cys 34 would not be conducive to deprotonation by the His were it present. For this reason, chain B and all the co-crystallizing waters were deleted from 2P92 so that modeling could be performed on monomer chain A The incomplete S. aureus Prp structure 2P92 Cys 34 was cycled through a library of rotamers and a conformation was chosen in which the sulfhydryl group was solvent-exposed. From this point, the loop between Gly 21 and Ile 32 could be modeled in such a way that the chain conformation would reflect the new pro-catalytic conformation of Cys 34. One loop model of the appropriate residues was built on this structure using a Modeller 9.12 (Sali, 1995) script written by Dr. Hardik Parikh (see Methods). The model now included all the residues present in chain A of S. aureus Prp, without chain interruption. This structure file and was then subjected to a loop refining script written by Dr. Parikh using Modeller to explore different possible conformations the loop missing from the crystal structure (Gly 21 to Ile 32). 50 models were created and checked for pro-catalytic conformations. A plausibly active model, number 12, was chosen based on the conformation of the relevant atoms of its catalytic residues (Cys: sulfur 254 and His: nitrogen 159 or nitrogen 161). In model 12, these atoms were only about 4 A apart. This distance would allow hydrogen bond formation and eventually proton abstraction from Cys 34, which is mandatory for catalysis in the mechanism of most cysteine proteases. In addition, this model gave the most open conformation of the upper loop, allowing greater solvent access to the catalytic center for the purpose of docking.

Model 12 was dimerized using 2P92 as a template and docking was attempted in Gold 5.2, but it became apparent that a four residue loop (residues 60-63), which would likely be mobile in solution, had a closed conformation that partially obscured the active site of the enzyme. For this reason, the loop refine script was again utilized in Modeller to allow the four residue loop to adopt a conformation that did not obscure the active site. 15 models were generated and the one that allowed greatest solvent exposure of the catalytic site was chosen.

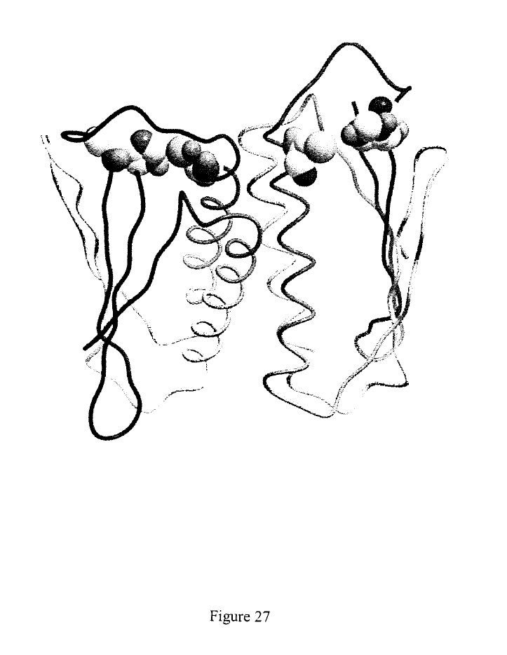

This refined model was dimerized in SYBYL because the crystal structure depicts a dimer and Prp has been found to dimerize in solution (Wall, Johnson, Christie and Peterson, in prep). The sulfhydryl hydrogen of the active site cysteine on chain A was replaced with a lone pair in SYBYL and the active site histidine was protonated for the purpose of docking (FIG. 27). The dimer model was read into Gold, and docking was attempted with the blocked peptide Acetyl (Ac)-Phe-Phe-Ala-Ser-Amide (Am), which represents the P2-P2′ substrate residues as they would appear within a peptide. Docking was achieved using a distance constraint of 3-2 Å from the catalytic sulfur to the carbonyl carbon of the peptide bond between Phe and Ala. This distance was used to adequately represent the beginning of the nucleophilic attack that would form the covalent intermediate between enzyme and substrate. The sidechains of the peptide substrate fit well into the hydrophobic pockets surrounding the enzyme catalytic center, so this substrate conformation was used as a scaffold constraint for the docking of a longer portion of the cleavage motif, Ac-Asn-Leu-Gln-Phe-Phe-Ala-Ser-Am. This was the largest peptide that was docked due to accuracy constraints on free peptide length. Residues Met 1, Leu 2, Lys 3 and Leu 4 from the full L27 cleavage motif do not appear in the model, though Leu 4 is quite conserved.

The overall docked peptide seemed to fit well, but the target scissile peptide bond in this model was angled such that the carbonyl carbon to be attacked was not in direct alignment with the thiolate nucleophile on the enzyme. Because this enzyme-substrate model was not perfectly positioned for catalysis, molecular dynamics simulations were used to “shake” the docked model into a more realistic catalytic conformation.

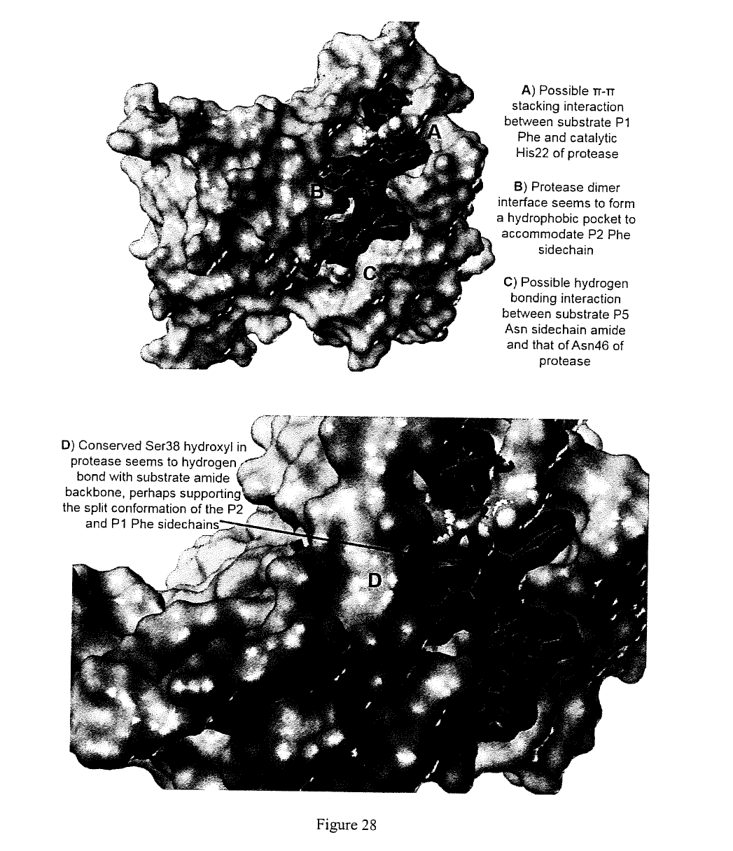

Molecular dynamics (MD) simulations were performed in NAMD (Phillips, 2005). Model 12_4_6 dimer with docked substrate Ac-NLQFFAS-Am was solvated with water and 50 mM NaCl ions, and subsequently 25,000 frames were collected for a total of 200 ns at 37° C. Frames from the early portion of this simulation showed improved steric interactions between enzyme and docked substrate—the MD simulation readjusted the target carbonyl so that nucleophilic attack from the enzyme thiolate could be possible. Frames from this re-positioned substrate enzyme complex were captured and are depicted in FIG. 28. As the MD simulation continued, the docked substrate began to loosen from the enzyme starting at its N-terminus, which seemed to be less tightly bound than the FAS region that was buried deeper in the enzyme active site. It may be that the earlier residues in the cleavage motif (MLKL; SEQ ID NO:37) have a large role in stabilization by binding to other regions of Prp. This model seems to predict a pi-pi stacking interaction of catalytic His 22 on the enzyme with the P1 Phe on the substrate (FIG. 28, 29). This is interesting because the intrinsic mobility of His 22, derived from its co-conservation with preceding Gly 21, would be problematic for the maintenance of an active catalytic center. If the substrate P1 Phe indeed stacks with His 22, this would probably confer stability to the active conformation of the enzyme in the presence of its appropriate substrate. In agreement with this hypothesis, the overwhelming majority of known L27 cleavage motifs have Phe as the P1 residue. Rare exceptions exist, however—Leu in C. difficile (cleavage motif MNLQLL′A; SEQ ID NO:38), and Cys in Clostridium acetobutylicum (extremely variant cleavage motif of INLSLC′A; SEQ ID NO:39). Indeed, the first member of this enzyme family that was identified as an active phage prohead protease, Prp in Streptococcus phage Cp-1, is published as cleaving an unrecognizable motif PVLEGARINH′A (SEQ ID NO:40), which still has a ring moiety as the P1 sidechain. However, the sequence of Prp in phage Cp-1 is extremely divergent from consensus. These disparities probably cannot be explained until there are more Prp structures from phage and bacteria with variant cleavage motifs.

The sidechain of the conserved Ser 38 residue on Prp seems to be a hydrogen bonding partner for the amide nitrogen between P2 Phe and P1 Phe in the substrate backbone. This residue is universally conserved among all Prps as a Ser or Thr spaced four residues after the catalytic Cys, making it the next rung on the alpha helix that faces the catalytic center. The completely conserved hydroxyl moiety seems to orient the amide nitrogen of the substrate peptide backbone in a way that promotes the split between the P2 and P1 Phe aromatic sidechains.

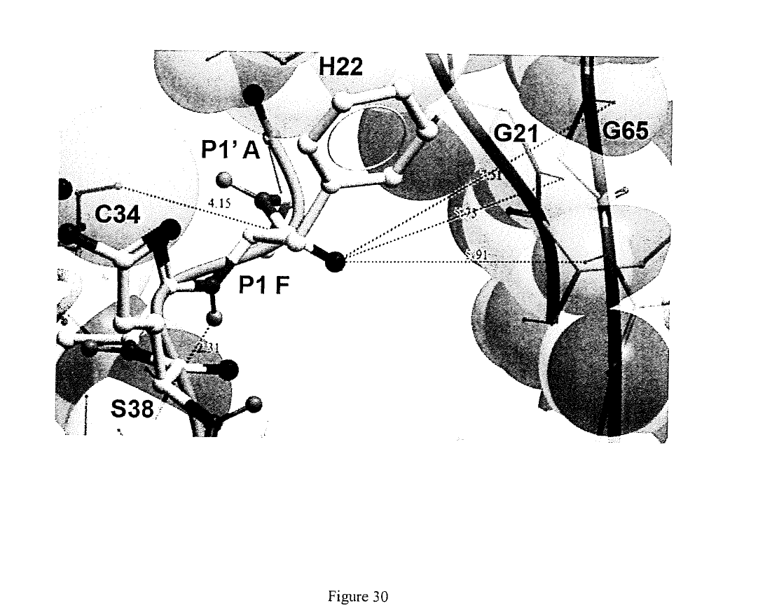

Two residues directly below the small loop on the right opposite the catalytic site seem to have quite an important role. This loop contains an extremely conserved glycine at position 65, which is adjacent to Gly 21, the completely conserved hinge that seems to promote His 22 movement. These two flexible glycines and the amide following Gly 65 create a fluctuating pocket that appears important for oxyanion stabilization during the tetrahedral intermediate stages of the mechanism of this protease (FIG. 30). The amide hydrogens would provide excellent attraction and support to the oxyanion to both stabilize its position and minimize its chance of erroneous bond formation. Further, they lie in a direct line passing through the substrate opposite but level with the catalytic Cys 34, positioning them perfectly to cradle the oxyanion after the linear nucleophilic attack. The fact that the oxyanion pocket seems so flexible is likely in keeping with the sequence specificity of this enzyme. In FIG. 30, the amide hydrogens are not all in a perfect cradling position relative to the substrate, but this could be an artifact of the original structure having been crystallized in the absence of ligand. Preliminary NMR studies have found evidence of massive structural rearrangements that occur upon substrate binding.

This model also appears to reveal the purpose of a previously unexplained Asn 46 conserved in the Firmicute Prps about 10 Å away from the catalytic site. This Asn would be an ideal hydrogen bonding partner for the conserved P5 Asn in the substrate cleavage motif. Preliminary work showed that Prp mutant N46A was significantly reduced in its ability to co-purify with tagged L27, suggesting a deficiency in binding. This Prp single mutant could still cleave its substrate, but kinetic studies should be performed to see whether the Km of this mutant enzyme is significantly shifted and inhibiting catalysis.

The P4 Leu in the cleavage motif fit well into a large hydrophobic patch on the exterior of the Prp subunit that was solvent-exposed in the crystal structure. It also appears that the initial residues of the N-terminal cleavage motif (MLKL; SEQ ID NO:37) would continue this pattern, filling in other hydrophobic patches on the Prp exterior that extend to the bottom rear of the other monomer.

In order to test the predictions that our model makes regarding residues important to substrate binding and catalysis and ultimately to search for inhibitors, it was necessary to design a high throughput-capable continuous fluorescent assay. This assay monitors Prp activity in the presence of a quenched fluorescent peptide containing the cleavage motif. In this type of assay, when the substrate is cleaved the fluorophore is released from its quenching partner and fluorescence readings at the fluorophore emission wavelength increase with time. There can be low level stable background fluorescence, but the majority of fluorescence should only be evolved in the presence of active Prp.

The Prp assay and completed preliminary tests that demonstrate that fluorescence is only produced in the presence of active Prp. The amino acid sequence of the substrate was selected to maximize its hydrophilic residue content, aiming for the greatest possible peptide solubility. The peptide is stable and did not self-cleave or change fluorescence over time when monitored in solution. The assay allows us to determine Prp kinetics, and the effects of any mutants on the Km or Kcat.

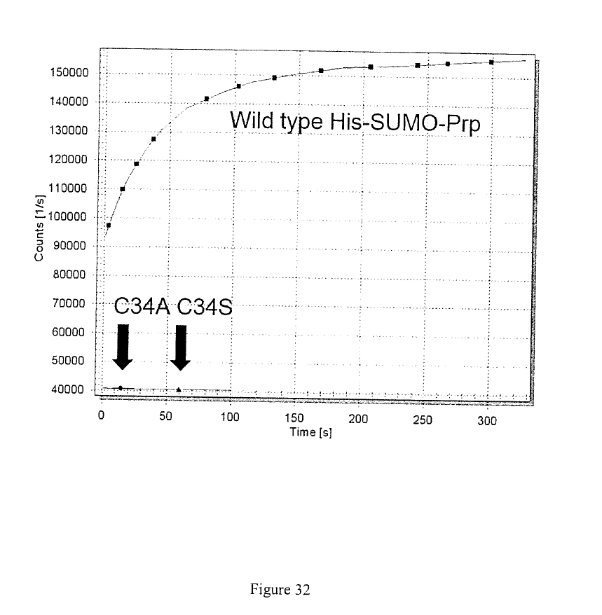

Prp was purified as an N-terminal His6-SUMO fusion protein and checked for activity using this assay. It was found that fluorescence increase only occurred in the presence of wild type Prp fusion proteins. Mutation of the catalytic Cys 34 to Ala renders Prp incapable of catalysis (see FIG. 8A and FIG. 8B.) In the presence of Prp C34A, no fluorescence increase was observed (FIG. 32). We also tested a Ser mutant of C34, in an attempt to discern whether the presence of a conserved Asp/Asn in the Prp catalytic motif suggested that the protease contained a catalytic triad. Though this mutant had been tested previously via co-expression with tagged L27 and Western blotting and found it to be inactive (FIG. 8B), we wanted to determine whether it could demonstrate any activity in vitro. The C34S active site mutant was also incapable of catalysis, indicating that the highly conserved Asp residue (Asp 31) is not sufficient to constitute a His-Ser-Asp catalytic triad in this protease. Of note, Prp C34A and C34S are both known to bind substrate tightly without cleaving, which demonstrates that binding is probably not sufficient to increase substrate fluorescence. This increases confidence in the validity of the Prp assay.

### Discussion

In this study, we have found that ribosomal protein L27 in Firmicutes and related bacteria has an N-terminal extension when compared to E. coli. This is important because the extension occludes N-terminal residues that have been well studied in E. coli L27 and found to be vital to A and P-site tRNA stabilization. We demonstrated that the extension must be cleaved away for S. aureus to survive and that the protease that performs the cleavage has a dominant negative effect when expressed as a catalytically inactive mutant. Overexpression of the inactive mutant gives rise to an aberrant pre-50S particle that contains some amount of the inactive protease in complex with un-cleaved L27, which would cause steric interference with the addition of later r-proteins like L16. This L27/Prp bound pair seems to also have blocked some L27 incorporation, as the mass spectrometry data suggest that L27 depleted from ribosomes on the whole. It seems from these data, however, that a non-functional, likely extremely deleterious pro-protein form of L27 can be assembled into the 50S. Importantly, not only is this L27 processing step essential, it is apparently highly regulated, because “pre-cleaved” L27□2-9 cannot complement an L27 deletion. This raises questions of whether the function of this process is related to ribosome biogenesis, regulation of translation, or perhaps both. The ribosomal biogenesis arrest produced in the presence of dominant negative Prp C34A is reminiscent of other studies of inactive ribosome assembly GTPase chaperones that bind but cannot dissociate.

Ribosomes are ancient machines—by far the largest group of genes in the Last Universal Common Ancestor (LUCA) are associated with translation (Fox, 2010). The ribosome is thought to have been nearly fully formed in LUCA, indicating that most of its development had occurred prior to that point in time, 3.5 billion years ago. While ribosomal structure and function are thought to be generally conserved across all domains of life, there are differences in composition that impact ribosomal size and function between eubacteria, archaea and eukaryotes and that are currently being exploited for therapeutic purposes. This new work demonstrates a novel twist in the story of bacterial ribosomes. Our findings indicate that there are aspects of the basic biology of the ribosome in S. aureus and related bacteria that differ substantially from that of the E. coli ribosome.

Ribosomal biology has been intensively studied in the Gram negative eubacterium E. coli, but is far less well understood in other bacteria. The information is largely limited to studies in E. coli and data from available crystal structures, of which there are only three for bacteria—E. coli, Thermus thermophilus and Deinococcus radiodurans. The latter two bacteria are hardly representative of the mesophile pathogens that we must attempt to target with antimicrobials. Nevertheless, the structure that is most often used to model ribosomal antibiotics is that of T. thermophilus, because it crystallizes readily. This is understandable, but at the same time we must acknowledge how this limits our view of ribosomal biology. There are good reasons to suspect that large differences exist in the ribosomes of many bacteria, especially those that contain the L27 N-terminal extension.

Work has been done in both E. coli (Eco) and Firmicute model organism Bacillus subtilis (Bsu) to understand the importance of each ribosomal protein to the function of the cell. Of fundamental note in these studies: the essential large subunit ribosomal proteins differ! There are some that are essential in Eco but dispensable in Bsu, including L22, L23, and L28. Those that are essential in Bsu but dispensable in Eco are L21, L24, L27, and L30 (Akanuma, 2012). Ribosomal protein complements also differ between bacteria. In a bioinformatic study of 995 bacterial genomes, 44 of 56 annotated r-proteins were found to be ubiquitous. Non-universal r-proteins L19p, L31p, L34p, L36p, L9p and S16p were missing from only one to three genomes examined. The rarest non-universal bacterial ribosomal proteins were determined to be L7ae, L25p, L30p, S21p, S22p and S31e, found in only a few groups (Yutin, 2012). It is not clear whether bacteria that encode L7ae (as noted, a homolog of Eukaryote and Archaeal L7) ever actually incorporate it into ribosomes. Though the gene for L7ae is present on the chromosome of B. subtilis, the L7ae protein was not detected in a proteomic study of fully assembled B. subtilis ribosomes (Lauber, 2009).

When r-proteins in each bacterial genome were concatenated and a phylogeny was drawn from this information, three major groups dubbed “megaphyla” emerged as deeply related clades of bacteria. Our interest was drawn to megaphylum III, which contains Fusobacteria, Mollicutes (aka Tenericutes), and Firmicutes. The Synergistetes were fairly closely related on a single offshoot of the tree (Yutin, 2012). All of these bacteria except one clade of Mollicutes contain the L27 N-terminal extension and Prp.

Mollicutes contain the smallest genomes of all bacteria that can be grown in axenic culture. Mycoplasma genitalium represents the iconic minimal “free-living” cellular genome at about 580,000 base pairs in length. Fully twenty-five percent of its genome is devoted to encoding genes related to translation. Further, the essential genes of this organism have been elucidated (Glass, 2006). Mollicutes can therefore be used as a reference for a postulated minimal essential translation apparatus (Grosjean, 2014). Four main subgroups of Mollicutes have been identified—Spiroplasma, Pneumoniae, Hominis and Acholeplasma Aneroplasma Candidatus phytoplasma (AAP). Of the culturable members of these groups, only the Hominis group lacks N-terminally extended L27 and the concomitant Prp homolog. If these minimal bacteria see fit to encode this system that requires what could be considered two “extra” pieces, therein lies a strong argument for that system's importance. The translational apparatus of the Mollicute groups also demonstrates that some of the most easily discarded genes belong to the class that modify ribosomal RNA and ribosomal proteins—of which Prp certainly would be one. Why then, is it different? There must be some reason for even the smallest of bacterial cells to retain this important cleavage event.

As the most discarded or shuffled translation genes are often rRNA and r-protein modification enzymes, it follows logically that ribosomal protein post-translational modifications differ widely. L27 has been mentioned extensively, but Bsu, unlike Eco, does not methylate its L3, doubly methylates its L16 and does not methylate its L33 (Lauber, 2009; Arnold, 1999). Many ribosomal biologists are starting to believe that the modifications themselves are not as important as the interaction of the modification enzyme with the ribosome at a given point. Frequently, if the nucleotide to be modified is mutated such that it cannot be methylated, lack of methylation at that site has no discernable effect on the function of the ribosome (Williamson, 2008). Simply the association of the modification enzyme at the right point in assembly may promote conformational changes in both enzyme and substrate that could chaperone the formation of complex assemblies (James R. Williamson, personal communication). Considering the data gathered thus far regarding the ability of inactive PrpC34A to inhibit ribosomal assembly, this hypothesis seems especially important.

Given this information it seems impossible that all bacterial ribosomes function in an identical manner, and they may even have significant conformational differences. Ribosomal protein processing is an unexplored mechanism that may provide new insights into the regulation of protein synthesis or possibly a new ordered pathway of ribosome assembly.

Almost all studies of ribosome assembly have been performed in E. coli, with only a few recent studies in B. subtilis. Ribosome assembly is a complex process, especially in the case of the large subunit. The production of rRNA is tightly coupled to growth rate and highly regulated. The ribosomal proteins, which must be produced in a concerted, stoichiometric manner, are often self-regulating, and many serve as repressors for the operon in which they are encoded (Shajani, 2011). Ribosomal proteins must bind their site on the nascent rRNA while it is being transcribed and simultaneously folded. This process is intermittently assisted by the binding and releasing of myriad rRNA modification enzymes, GTPases, and at least 20 to 30 other chaperones. Though it has often been modeled as an extremely hierarchical and step-wise progression, the sum of these parallel events is a largely stochastic and statistical process that results in many convergent pathways to assembly (Williamson, 2008; Sykes, 2010).

The in vitro assembly of a functional 50S subunit of the E. coli ribosome by Nierhaus and Dohme in 1974 was met with deserved acclaim, but the conditions under which assembly was performed were harsh and nonphysiological (Nierhaus, 1974). Nevertheless, the ordered, linear Nierhaus Map of 50S subunit assembly has been cited by those studying bacterial ribosomes ever since. In vitro large subunit assembly consists of three kinetic intermediates: a 33S particle, a 41S to 43S particle and a 48S particle. The in vivo assembly intermediates discovered are of similar sedimentation rates, but their ordered introduction of ribosomal proteins is quite different. For instance, late 33S or 41S in vitro particles contain L2, leading to its classification as an early-binding protein (Nierhaus, 1974). However, E. coli in vivo 32S and 43S large subunit precursors were found not to contain L2, indicating that it was a middle to late-binding protein (Nierhaus, 1991). Study of the assembly of the 50S subunit is particularly problematic, especially when compared to the well-characterized 30S subunit. Many types of local secondary structure occur during transcription of the 23S rRNA, and it is not yet understood how all of these are chaperoned and coaxed into an active complex. One of the roles r-proteins seem to perform is to effect the gradual decrease of rRNA flexibility, guiding the formation of single stranded secondary structures until an active particle is achieved. There are also many RNases involved in assembly, most of which are not understood. The in vitro work also certainly does not account for the truly massive cadre of ribosomal GTPases, methylases and other possible chaperones that are extremely conserved, some of them across all domains of life (Shajani, 2011).

Recently, investigators at The Scripps Research Institute, Michigan State and McMaster University have employed the increased precision of modern mass spectrometry to analyze large subunit in vivo assembly intermediates and determine their protein content. Current methods of stable isotopic labeling and quantitative mass spectrometry (qMS) can serve to analyze both rRNA and proteins and check for modification of those components and to examine their relative order of addition to the ribosome (Williamson, 2008).

To inventory and quantitate ribosomal proteins from in vivo assembly intermediates, two cultures of E. coli were grown, one in 14N media, the other in 15N media. All the ribosomal subunits and assembly intermediates gathered from the 14N culture were fractionated after sucrose gradient centrifugation. Each fraction was spiked with the same amount of exclusively fully assembled 70S ribosomes gathered from the 15N culture. In this way, the spectrum of each partial assembly 14N peptide could be compared in absolute area and intensity with the spectrally shifted fully assembled 15N 70S standard peptide (Chen, 2013).

Isotopic pulse labeling studies involve cells that are harvested from normal media and grown in 50% 15N media for various time points. When these fractions are compared to a fully 15N labeled 70S ribosome standard, lighter, unlabeled peptide peaks represent r-proteins incorporated before the pulse and 50% labeled peaks represent r-proteins incorporated after the pulse (Sykes, 2010). This method was used to distinguish “on pathway” intermediates from degradation products by how quickly the label is incorporated into all proteins in a particular particle, as degradation products would be less likely to incorporate label over time. Particle labeling kinetics can be further subdivided into peptide labeling kinetics. The ribosomal proteins that are labeled fastest are the ones that were first translated after the pulse. It has already been shown that free ribosomal protein pools are not large for early assembly proteins, so the majority of those have already been incorporated into a particle without label and require a second ribosomal generation to become labeled. In contrast, late assembly proteins are produced and added to existing unlabeled assembly intermediates, demonstrating faster label uptake (Chen, 2013).

These isotopic labeling studies in E. coli revealed a more complex assembly landscape for the 50S subunit than previously imagined. 50S assembly is more diffuse and nuanced than 30S assembly, which forms a less complex particle that consists chiefly of the decoding center. Two regions in the large 50S subunit must be assembled with great accuracy—the exit tunnel and the PTC, compared to only one region in the smaller 30S subunit. Assembly of the 50S subunit takes twice as long as the 30S, and appears to start at the “back”—the region on the opposite side of the PTC, above the exit tunnel. There is an intermediate step in which r-proteins are inserted in various areas all over the surface of the particle, and assembly then ends with construction of the central protuberance and the PTC. Large subunit r-protein labeling kinetics were compared and grouped temporally. With the exception of the proteins that bind to the 5′ end of the 23 S rRNA transcript (L20, L21, L22 and L24), large subunit proteins bind every region of the rRNA at every stage of assembly. This study led to the re-classification of L27 as a late-binding protein, though Nierhaus found it to be middle-binding in vivo in 1991. In addition to r-protein temporal assembly designations, this study uncovered studied and hitherto unknown GTPases and chaperones that likely assist in the assembly of the large subunit (Chen, 2013). What is known about the roles that ribosomal chaperones play will be discussed later in this work.

When in vivo isotopic labeling studies were performed in B. subtilis at Michigan State University and McMaster University in collaboration with J. R. Williamson at Scripps, an RbgA (large subunit assembly GTPase not present in E. coli) depleted cell produced a 45S particle somewhat similar in r-protein composition to the aberrant particle in this work. Both particles lost staggering amounts of L16, L25 and L27 and some of L18, all proteins resident on or near the central protuberance. There were some notable differences, however—our particle was conspicuously deficient in L29, and had normal levels of L30, while theirs had these ratios reversed. While their particle was deficient in L27 it had normal levels of L20, yet we lost significant amounts of L20 in our L27-deficient particle. As previously mentioned, L27 deletion in E. coli depletes the large subunit of L20 and L21, yet L21 was within normal range for our particle while L20 was significantly depleted. Many of these discrepancies might be corrected with increased accuracy in our measurement of our r-protein peptides, which will require some type of peptide labeling for qMS. Taking into account the limited scope of data that we have, it seems likely that we have trapped an earlier assembly intermediate than the one studied by this group, simply because many more r-proteins intrinsic to the interior of the ribosome were deficient in our particle compared to theirs (Jomaa, 2013).

When the B. subtilis 45S particles were subjected cryo-electron microscopy (cryo-EM), it was found that the central protuberance and PTC were the least mature regions, with disorder in the sites that should be open to accept tRNAs. These particles were incompetent for translation, but could be matured when both RbgA and L16 are reintroduced. The authors of this study theorized that the final RbgA effect, which seems to allow L16 incorporation and support other late r-protein association, was a checkpoint on PTC formation, only permitting the active site of the ribosome to mature as long as the rest of the ribosome was assembled correctly (Jomaa, 2013).

As a further illustration of the confusion in the literature surrounding ribosome assembly in B. subtilis, a paper published immediately before that of Jomaa et al 2013 used post growth isobaric tagging methods for qMS (as opposed to in vivo isotope labeling) on B. subtilis r-proteins from 45S subunits in the same RgbA depletion strain utilized by Jomaa et al. The study by Li et al. in 2013 measured extremely dissimilar ribosomal protein ratios and significantly different cryo-electron microscopy images, with some particles from their 45S pool showing absolutely no density for the CP, even though their aberrant particles arose from the same strain background with similar ribosome purification methods. I noted only three substantial methods deviations that might account for these differences. The first difference, as mentioned above, was the use of in vitro isobaric tag labeling instead of in vivo stable isotope incorporation. In vitro labeling is reported to be less accurate because it relies on successful conjugation reactions between the isobaric tags and the peptides rather than assured label incorporation via growth with an isotope tag as the sole source of nitrogen. Further, degradation products cannot be distinguished from on pathway intermediates using isobaric tag labeling (Chen, 2013). Second, wild type 50S control subunits in the Li et al. 2013 study were collected from lab strain B. subtilis 168, not the RgbA depletion strain background in RgbA replete conditions as in Jomaa et al. Third and most interestingly, the 45S immature subunits collected from RgbA depleted cells in the Li et al study were subjected to an extra purification step involving the addition of large amounts of wild-type 30S subunits to the 45S fraction and incubating for ten minutes at 37° C., allowing subunits to associate if possible, and then subjecting that particle mixture to another sucrose gradient fractionation to re-isolate the 45S subunits. Ostensibly this would clear the 45S peak of any contaminating mature 50S particles, or for that matter, any particles that retained functional vestiges of the intersubunit bridges required for stable association with the 30S, by moving those complexes to the 70S peak.

The particles reported by Li et al. 2013 showed measurable loss of L3, L15 and L13, setting them at odds with the data set in this work and that of Jomaa et al. These proteins have not been reported to be involved in intersubunit bridging, so their disappearance is mysterious. The fact and extent of the losses of L18 and L20 that were observed disagree with both Jomaa and Li. There was a group of proteins that were consistently lost or could not be detected in all of the studies, this work included: L28, L16, L27, L33, L35 and L36. While our methods of quantification were primitive compared to the other studies, their consistency with other more precise methods in identification of these proteins was reassuring. There are very likely well-defined groups of r-proteins added at different stages of assembly, but the interconnectedness and dynamic nature of the ribosome significantly complicates interpretation of these groups. Later proteins stabilize earlier proteins and all participate in massive rRNA rearrangements during ribosome biogenesis—effects the loss of just one protein can cause major conformational shifts tens of angstroms distant from the location of the deficit. It is clear that much work remains to be done using qMS, improved cryo electron microscopy resolution and hitherto unexplored avenues to uncover the complexities of bacterial ribosome assembly, especially in organisms that are not E. coli.

The type of in vivo isotopic labeling study performed in Chen et al, 2013 and Jomaa et al, 2013 has worked well with B. subtilis, yet in its current form the procedure cannot be performed with S. aureus, which cannot grow well (if at all) in minimal media. The use of stable isotope labeled amino acids in culture (SILAC) reagents to label r-proteins in S. aureus grown in defined media, while theoretically possible, is prohibitively expensive. It would cost about one thousand dollars per liter of media to incorporate only a few labeled amino acids, while complete amino acid labeling comparable to the nuanced effects achieved in the above studies would cost multiple thousands of dollars per liter. Other chemical labeling methods such as isobaric tagging for relative and absolute quantification (iTRAQ) tags must be applied to S. aureus ribosomal samples after their collection and before mass spectrometric analysis, rather than labeling in vivo. iTRAQ tags fragment at offset masses, shifting a peptide to a higher mass than its counterpart in the control sample, so peaks may be compared. As mentioned previously, these methods are less accurate than pulse labeling with stable isotopes. It is probably a great deal more practical for extensive ribosome assembly studies in Firmicutes to be performed in B. subtilis, which thrives in minimal media and is also easier to lyse given its elongated cell morphology compared to spherical S. aureus.

In order to understand possible functions of the Prp/long L27 cleavage system, it is helpful to examine existing ribosome associated proteins known to be involved in assembly or ribosomal regulation and their possible correlations with Prp/long L27. There are many, many documented proteins that have been observed to interact with the ribosome in various ways; they have been reviewed extensively (Iost, 2013; Goto, 2013; Wilson, 2007). As previously mentioned, Mollicutes can be considered the model for a minimal translation apparatus due to their small genomes (Grosjean, 2014), making retention of the Prp/long L27 system in many clades seem all the more significant. We can compare and contrast the ribosome-associated chaperones and regulators present in Mollicutes with and without Prp/long L27 to provide focus on what might be different and important to the ribosomes and ribosomal pathways in our particular group of bacteria. Perhaps the pattern of ribosomal chaperone conservation within this group will eventually give clues to the reason that the Prp/long L27 system is conserved.

Ribosomes require a number of trans-acting factors that use GTP or ATP to engage in energy consuming structural rearrangements that are required for correct rRNA folding during maturation. These enzymes generally have one conformation that has high ribosomal affinity when bound to GTP, and then a radically different conformation after GTP hydrolysis that has considerably less affinity for the ribosome (Goto, 2013). RgbA, previously discussed in the section on ribosomal assembly, contains a GTPase domain that is conserved across all three forms of life and is involved in a late step of 50S particle maturation. Depletion of RgbA leads to decreased 70S ribosomes relative to subunits, disorder in the central protuberance of the 45S particle produced and loss of late binding r-proteins, such as L16, L28, L27, L36 and L33a (Jomaa, 2013; Li, 2013). Recently, it has been demonstrated that a function of RbgA seems to be to properly position r-protein L6 during assembly, as mutations in RbgA that caused ribosome assembly defects were rescued by compensatory mutations in L6. Though they did not affect cellular growth rate, these L6 mutants still caused some disturbance in 50S assembly. From this and the locations of the L6 mutants on a well-conserved L6/rRNA interface, the authors theorize that RgbA associates with L6 and helix 97 in order to properly position them, and/or to allow incoming late assembly proteins to bind (Gulati, 2014).

This GTPase is universal, and there is so far no recognized ribosomal GTPase differentially conserved in Prp/long L27 Mollicutes vs other Mollicutes. It is therefore less likely that RgbA is involved in the pathway created by Prp/L27, but understanding its role serves to highlight the function of ribosomal chaperones that reconfigure the assembling structure in favor of proper protein placement. Prp might have a similar function, associating with the ribosome by binding to the L27 N-terminal extension, allowing conformational changes to occur around it or effecting them directly, and then cleaving away the small peptide in order to dissociate from the assembling ribosome. Indeed, this reaction requires no ATP or GTP, possibly making it an energy efficient way to solve a kinetic problem that occurs in rRNA folding or r-protein incorporation.

DEAD-box helicases are widely conserved throughout all domains of life and essential in eukaryotes; in complex cells their function is to chaperone single stranded RNA and thus facilitate almost every aspect of RNA metabolism and transport. In contrast, the functions of studied bacterial homologs are poorly understood, and the proteins are usually only essential under cold shock conditions. In E. coli and Firmicutes, DEAD-box helicases are highly associated with ribosome biogenesis and RNA metabolism. The protein known as DeaD or CsdA in E. coli has been shown to aid in the assembly of the 50S subunit. Strains with deaD deletions grow slowly, form aberrant 40S precursors under normal growing conditions, and the majority of their mature 50S subunits are defective in translation, indicating that DeaD might have a role in the final steps of ribogenesis. At low temperatures DeaD co-sediments with the 50S subunit, suggesting that it might have a role in preventing undesirable rRNA secondary structure that occurs under those conditions. In Firmicutes the DeaD homologs are also non-essential at 37° C., but become essential under cold shock conditions (lost, 2013). Studies of Firmicute DeaD effects on ribogenesis are sparse, but DeaD deletions were analyzed for ribosomal anomalies in Listeria monocytogenes to understand its psychrotolerance mechanisms. These studies demonstrated that the deletion caused a loss of 70S subunits and accumulation of a 40S precursor at 16° C. Further, the authors demonstrated via complementation that DeaD binds the 50S subunit and fulfills its role as a chaperone via its C-terminus, as the 40S precursor could be ablated by complementing with wild-type DeaD but not a C-terminal deletion mutant (Netterling, 2012). Interestingly, even though Mollicutes show very low overall conservation of DEAD-box helicases, the Prp/long L27 group universally retains a protein highly related to DeaD, while the Hominis group that lacks Prp/long L27 does not (Grosjean, 2014).

Bacterial rRNA is produced as a precursor molecule that must be trimmed by Rnase III or a functional homolog to begin the formation of mature 23S, 16S and 5S molecules. In contrast to 16S rRNA, which after Rnase III cleavage begins as 17S rRNA and undergoes fairly extensive processing, in E. coli precursor 23S rRNA loses only a few bases during maturation, seven from the 5′ end and eight from the 3′ end. Yet, it was recently shown in that retention of the eight nucleotides on the 3′ end caused a significant defect in ribosome assembly (Gutgsell, 2012). Processing of 23S rRNA occurs via Mini Rnase III or alternatively Rnase J1, J2, Ph and YhaM in B. subtilis (Redko, 2010). Mycoplasma genetialium primarily utilizes Rnase R for maturation of its 23S rRNA. In this system Rnase R performs various precise methylation-sensitive cleavages of rRNA and executes efficient tRNA processing (Lalonde, 2007). Mollicutes have Rnase III homologs, but no Rnase Ph or YhaM. They also do not contain Rnase A, B, D, E, G, HI, HII or T, but Mollicutes that can be grown in axenic culture uniformly retain Rnase R, J1, J2, P, and Nrna, a ribonuclease specific to very short RNAs. However, Prp/long L27 group Mollicutes retain another ribonuclease, Rnase Y, that is not present in Mollicutes without the L27 cleavage system (Grosjean, 2014). Rnases can be involved in the processing of RNAs to maturation and also in the RNA metabolism of the cell. Regulation of certain Rnases can stabilize or destabilize existing transcripts, allowing for global cellular response to stimulus.

Ribonuclease M5 is responsible for 5S rRNA maturation in low G-C Gram positive bacteria that do not encode the RNase E/G homolog that performs that role in the proteobacteria. Ribonuclease M5 and 5S rRNA maturation have been shown to be nonessential in B. subtilis (Condon, 2001; Allemand, 2011), yet Prp/long L27 group Mollicutes retain this processing enzyme while Hominis group Mollicutes without Prp/long L27 do not (Grosjean 2014). Further study reveals that while it contains an expected conserved domain with resemblance to an N-terminal topoisomerase-primase found in DnaG homologs (NCBI cd01027, with relationship to pfam domain 13155), the M5 nuclease C-terminal domain DUF4093 is of unknown function. This DUF is conserved in Firmicutes, Mollicutes in the groups that contain Prp/long L27, Fusobacteria and Spirochaetes. The domain has not been found to be conserved in Synergistes, however, which have Rnase E homologs instead of ribonuclease M5, making retention of this DUF not perfectly correlative with the presence of Prp or Prp/long L27. Yet, the interesting dichotomy of Mollicute Ribonuclease M5 conservation in those that contain Prp/L27 versus its loss in those that do not suggests marked differences in their rRNA maturation pathways.

Apart from ribosome assembly, it seems theoretically possible that the amino acid starvation response could cause down-regulation of Prp, causing buildup of long L27 and arresting synthesis of new ribosomes. Bacteria are known to regulate production of their ribosomes in response to nutrient limitation in part by controlling the transcription of rRNA. In E. coli, this is a well-studied phenomenon called the stringent response, and its main effectors are RelA, SpoT and pyrophosphoryated GTP or GDP, abbreviated (p)ppGpp. (p)ppGpp uses protein DksA as a cofactor to exert widespread changes in transcriptional regulation by binding to RNA polymerase (RNAP). The stringent response also affects translation and replication. This phenomenon has been intensively studied for over forty years, yet the exact mechanism (p)ppGpp uses to exert its effect remains uncertain, and its binding site on RNAP remains unknown (Wolz, 2010).

Recent work has uncovered that proteobacteria like E. coli are distinct from Firmicutes like S. aureus in their (p)ppGpp production pathway and its regulatory targets. There is no homolog of DksA in Firmicutes, no indication that (p)ppGpp interacts directly with RNA polymerase, and concomitantly the region before the transcriptional start for rRNA operons does not contain the discriminator sequences that would decrease their transcription, as in the E. coli (p)ppGpp/DksA/RNAP response system. Instead of RelA ((p)ppGpp production) and SpoT ((p)ppGpp hydrolysis), S. aureus contains a fusion protein with hydrolase and synthase domains called Rsh and two small (p)ppGpp synthases often called RelP and Rel Q (Wolz, 2010). It appears that the function of RelP/Q is to produce small amounts of background (p)ppGpp during exponential phase, which causes the hydrolase domain of Rsh to be essential for consistent low level detoxification. Rsh responds to amino acid stress while RelP/Q are more responsive to cell wall stress or alkaline conditions (Geiger, 2014). Much work remains to be done to understand the (p)ppGpp regulon in Firmicutes, including response regulator CodY and virulence up-regulation under stress conditions, which are beyond the scope of this dissertation.

In the Hominis group of Mollicutes that do not contain Prp/long L27, there is no obvious (p)ppGpp synthase homolog to be found anywhere in the genome, so it is possible that this GTP nucleotide regulatory system does not even exist in that group of bacteria. In contrast, the Prp/long L27 Mollicutes uniformly retain the bifunctional Rsh protein, but do not retain the smaller RelP/Q (p)ppGpp synthases (Grosjean, 2014).

### REFERENCES

Throughout this application, various references describe the state of the art to which this invention pertains. The disclosures of these references are hereby incorporated by reference into the present disclosure.
Akanuma, G., Nanamiya, H., Natori, Y., Yano, K., Suzuki, S., Omata, S., . . . Kawamura, F. (2012). Inactivation of ribosomal protein genes in bacillus subtilis reveals importance of each ribosomal protein for cell proliferation and cell differentiation. Journal of Bacteriology, 194(22), 6282-6291. doi:10.1128/JB.01544-12 [doi]
Aksyuk, A. A., & Rossmann, M. G. (2011). Bacteriophage assembly. Viruses, 3(3), 172-203. doi:10.3390/v3030172
Anderson, K. L., Roberts, C., Disz, T., Vonstein, V., Hwang, K., Overbeek, R., . . . Dunman, P. M. (2006). Characterization of the staphylococcus aureus heat shock, cold shock, stringent, and SOS responses and their effects on log-phase mRNA turnover. Journal of Bacteriology, 188(19), 6739-6756. doi:10.1128/JB.00609-06
Anderson, K. L., Roux, C. M., Olson, M. W., Luong, T. T., Lee, C. Y., Olson, R., & Dunman, P. M. (2010). Characterizing the effects of inorganic acid and alkaline shock on the staphylococcus aureus transcriptome and messenger RNA turnover. FEMS Immunology and Medical Microbiology, 60(3), 208-250. doi:10.1111/j.1574-695X.2010.00736.x; 10.1111/j.1574-695X.2010.00736.x
Arnold, R. J., & Reilly, J. P. (1999). Observation of escherichia coli ribosomal proteins and their posttranslational modifications by mass spectrometry. Analytical Biochemistry, 269(1), 105-112. doi:S0003-2697(98)93077-9 [pii]
Auerbach, T., Bashan, A., Harms, J., Schluenzen, F., Zarivach, R., Bartels, H., . . . Yonath, A. (2002). Antibiotics targeting ribosomes: Crystallographic studies. Current Drug Targets. Infectious Disorders, 2(2), 169-186.
Ban, N., Beckmann, R., Cate, J. H., Dinman, J. D., Dragon, F., Ellis, S. R., . . . Yusupov, M. (2014). A new system for naming ribosomal proteins. Current Opinion in Structural Biology, 24, 165-169. doi:10.1016/j.sbi.2014.01.002 [doi]
Bashan, A., & Yonath, A. (2008). Correlating ribosome function with high-resolution structures. Trends in Microbiology, 16(7), 326-335. doi:10.1016/j.tim.2008.05.001
Becher, D., Hempel, K., Sievers, S., Zuhlke, D., Pane-Farre, J., Otto, A., . . . Hecker, M. (2009). A proteomic view of an important human pathogen—towards the quantification of the entire staphylococcus aureus proteome. PloS One, 4(12), e8176. doi:10.1371/journal.pone.0008176; 10.1371/journal.pone.0008176
Belousoff, M. J., Davidovich, C., Zimmerman, E., Caspi, Y., Wekselman, I., Rozenszajn, L., . . . Yonath, A. (2010). Ancient machinery embedded in the contemporary ribosome. Biochemical Society Transactions, 38(2), 422-427. doi:10.1042/BST0380422; 10.1042/BST0380422
Castro, H. C., Abreu, P. A., Geraldo, R. B., Martins, R. C., dos Santos, R., Loureiro, N. I . . . Rodrigues, C. R. (2011). Looking at the proteases from a simple perspective. Journal of Molecular Recognition: JMR, 24(2), 165-181. doi:10.1002/jmr.1091 [doi]
Cech, T. R. (2000). Structural biology. the ribosome is a ribozyme. Science (New York, N.Y.), 289(5481), 878-879.
Chaudhuri, R. R., Allen, A. G., Owen, P. J., Shalom, G., Stone, K., Harrison, M., . . . Charles, I. G. (2009). Comprehensive identification of essential staphylococcus aureus genes using transposon-mediated differential hybridisation (TMDH). BMC Genomics, 10, 291-2164-10-291. doi:10.1186/1471-2164-10-291; 10.1186/1471-2164-10-291
Chirgadze, Y. N., Clarke, T. E., Romanov, V., Kisselman, G., Wu-Brown, J., Soloveychik, M., . . . Chirgadze, N. Y. (2015). The structure of SAV1646 from staphylococcus aureus belonging to a new ‘ribosome-associated’ subfamily of bacterial proteins. Acta Crystallographica. Section D, Biological Crystallography, 71(Pt 2), 332-337. doi:10.1107/S1399004714025619 [doi]
Colca, J. R., McDonald, W. G., Waldon, D. J., Thomasco, L. M., Gadwood, R. C., Lund, E. T., . . . Mankin, A. S. (2003). Cross-linking in the living cell locates the site of action of oxazolidinone antibiotics. The Journal of Biological Chemistry, 278(24), 21972-21979. doi:10.1074/jbc.M302109200
Condon, C., Brechemier-Baey, D., Beltchev, B., Grunberg-Manago, M., & Putzer, H. (2001). Identification of the gene encoding the 5S ribosomal RNA maturase in bacillus subtilis: Mature 5S rRNA is dispensable for ribosome function. RNA (New York, N.Y.), 7(2), 242-253.
D'Elia, M. A., Pereira, M. P., Chung, Y. S., Zhao, W., Chau, A., Kenney, T. J., . . . Brown, E. D. (2006). Lesions in teichoic acid biosynthesis in staphylococcus aureus lead to a lethal gain of function in the otherwise dispensable pathway. Journal of Bacteriology, 188(12), 4183-4189. doi:10.1128/JB.00197-06
Deu, E., Verdoes, M., & Bogyo, M. (2012). New approaches for dissecting protease functions to improve probe development and drug discovery. Nature Structural & Molecular Biology, 19(1), 9-16. doi:10.1038/nsmb.2203 [doi]
Duda, R. L., Oh, B., & Hendrix, R. W. (2013). Functional domains of the HK97 capsid maturation protease and the mechanisms of protein encapsidation. Journal of Molecular Biology, 425(15), 2765-2781. doi:10.1016/j.jmb.2013.05.002 [doi]
Fetrow, J. S. (2006). Active site profiling to identify protein functional sites in sequences and structures using the deacon active site profiler (DASP). Current Protocols in Bioinformatics/Editorial Board, Andreas D. Baxevanis . . . [Et Al.], Chapter 8, Unit 8.10. doi:10.1002/0471250953.bi0810s14 [doi]
Fox, G. E. (2010). Origin and evolution of the ribosome. Cold Spring Harbor Perspectives in Biology, 2(9), a003483. doi:10.1101/cshperspect.a003483; 10.1101/cshperspect.a003483
Frank, J., & Agrawal, R. K. (2000). A ratchet-like inter-subunit reorganization of the ribosome during translocation. Nature, 406(6793), 318-322. doi:10.1038/35018597 [doi]
Fraser, C. M., Gocayne, J. D., White, O., Adams, M. D., Clayton, R. A., Fleischmann, R. D., . . . Venter, J. C. (1995). The minimal gene complement of mycoplasma genitalium. Science (New York, N.Y.), 270(5235), 397-403.
Fuchs, S., Pane-Farre, J., Kohler, C., Hecker, M., & Engelmann, S. (2007). Anaerobic gene expression in staphylococcus aureus. Journal of Bacteriology, 189(11), 4275-4289. doi:10.1128/JB.00081-07
Fuchs, S., Zuhlke, D., Pane-Farre, J., Kusch, H., Wolf, C., Reiss, S., . . . Engelmann, S. (2013). Aureolib—a proteome signature library: Towards an understanding of staphylococcus aureus pathophysiology. PloS One, 8(8), e70669. doi:10.1371/journal.pone.0070669; 10.1371/journal.pone.0070669
Garcia-Vallve, S., Simo, F. X., Montero, M. A., Arola, L., & Romeu, A. (2002). Simultaneous horizontal gene transfer of a gene coding for ribosomal protein 127 and operational genes in arthrobacter sp. Journal of Molecular Evolution, 55(6), 632-637. doi:10.1007/s00239-002-2358-5
Geiger, T., & Wolz, C. (2014). Intersection of the stringent response and the CodY regulon in low GC gram-positive bacteria. International Journal of Medical Microbiology: IJMM, 304(2), 150-155. doi:10.1016/j.ijmm.2013.11.013 [doi]
Gibson, D. G., Young, L., Chuang, R. Y., Venter, J. C., Hutchison, C. A., 3rd, & Smith, H. O. (2009). Enzymatic assembly of DNA molecules up to several hundred kilobases. Nature Methods, 6(5), 343-345. doi:10.1038/nmeth.1318; 10.1038/nmeth.1318
Glass, J. I., Assad-Garcia, N., Alperovich, N., Yooseph, S., Lewis, M. R., Maruf, M., . . . Venter, J. C. (2006). Essential genes of a minimal bacterium. Proceedings of the National Academy of Sciences of the United States of America, 103(2), 425-430. doi:10.1073/pnas.0510013103
Goto, S., Muto, A., & Himeno, H. (2013). GTPases involved in bacterial ribosome maturation. Journal of Biochemistry, 153(5), 403-414. doi:10.1093/jb/mvt022 [doi]
Gould, I. M. (2009). Antibiotic resistance: The perfect storm. International Journal of Antimicrobial Agents, 34 Suppl 3, S2-5. doi:10.1016/S0924-8579(09)70549-7; 10.1016/S0924-8579(09)70549-7
Gulati, M., Jain, N., Davis, J. H., Williamson, J. R., & Britton, R. A. (2014). Functional interaction between ribosomal protein L6 and RbgA during ribosome assembly. PLoS Genetics, 10(10), e1004694. doi:10.1371/journal.pgen. 1004694 [doi]
Gutgsell, N. S., & Jain, C. (2012). Gateway role for rRNA precursors in ribosome assembly. Journal of Bacteriology, 194(24), 6875-6882. doi:10.1128/JB.01467-12 [doi]
Harms, J., Schluenzen, F., Zarivach, R., Bashan, A., Gat, S., Agmon, I., . . . Yonath, A. (2001). High resolution structure of the large ribosomal subunit from a mesophilic eubacterium. Cell, 107(5), 679-688. doi:S0092-8674(01)00546-3 [pii]
Hughes, D., & Karlen, A. (2014). Discovery and preclinical development of new antibiotics. Upsala Journal of Medical Sciences, doi:10.3109/03009734.2014.896437
Humphrey, W., Dalke, A., & Schulten, K. (1996). VMD: Visual molecular dynamics. Journal of Molecular Graphics, 14(1), 33-8, 27-8. doi:0263785596000185 [pii]
Iost, I., Bizebard, T., & Dreyfus, M. (2013). Functions of DEAD-box proteins in bacteria: Current knowledge and pending questions. Biochimica Et Biophysica Acta, 1829(8), 866-877. doi:10.1016/j.bbagrm.2013.01.012 [doi]
Ji, Y., Zhang, B., Van, S. F., Horn, Warren, P., Woodnutt, G., . . . Rosenberg, M. (2001). Identification of critical staphylococcal genes using conditional phenotypes generated by antisense RNA. Science (New York, N.Y.), 293(5538), 2266-2269. doi:10.1126/science. 1063566
Jomaa, A., Jain, N., Davis, J. H., Williamson, J. R., Britton, R. A., & Ortega, J. (2014). Functional domains of the 50S subunit mature late in the assembly process. Nucleic Acids Research, 42(5), 3419-3435. doi:10.1093/nar/gkt1295 [doi]
Kearse, M., Moir, R., Wilson, A., Stones-Havas, S., Cheung, M., Sturrock, S., . . . Drummond, A. (2012). Geneious basic: An integrated and extendable desktop software platform for the organization and analysis of sequence data. Bioinformatics (Oxford, England), 28(12), 1647-1649. doi:10.1093/bioinformatics/bts199; 10.1093/bioinformatics/bts199
Klein, E., Smith, D. L., & Laxminarayan, R. (2007). Hospitalizations and deaths caused by methicillin-resistant staphylococcus aureus, united states, 1999-2005. Emerging Infectious Diseases, 13(12), 1840-1846. doi:10.3201/eid1312.070629; 10.3201/eid1312.070629
Kobayashi, K., Ehrlich, S. D., Albertini, A., Amati, G., Andersen, K. K., Arnaud, M., . . . . Ogasawara, N. (2003). Essential bacillus subtilis genes. Proceedings of the National Academy of Sciences of the United States of America, 100(8), 4678-4683. doi:10.1073/pnas.0730515100
Kokkori, S., Apostolidi, M., Tsakris, A., Pournaras, S., Stathopoulos, C., & Dinos, G. (2014). Linezolid-dependent function and structure adaptation of ribosomes in a staphylococcus epidermidis strain exhibiting linezolid dependence. Antimicrobial Agents and Chemotherapy, 58(8), 4651-4656. doi:10.1128/AAC.02835-14 [doi]
Kreiswirth, B. N., Lofdahl, S., Betley, M. J., O'Reilly, M., Schlievert, P. M., Bergdoll, M. S., & Novick, R. P. (1983). The toxic shock syndrome exotoxin structural gene is not detectably transmitted by a prophage. Nature, 305(5936), 709-712.
Kunst, F., Ogasawara, N., Moszer, I., Albertini, A. M., Alloni, G., Azevedo, V., . . . Danchin, A. (1997). The complete genome sequence of the gram-positive bacterium bacillus subtilis. Nature, 390(6657), 249-256. doi:10.1038/36786
Lalonde, M. S., Zuo, Y., Zhang, J., Gong, X., Wu, S., Malhotra, A., & Li, Z. (2007). Exoribonuclease R in mycoplasma genitalium can carry out both RNA processing and degradative functions and is sensitive to RNA ribose methylation. RNA (New York, N.Y.), 13(11), 1957-1968. doi:rna.706207 [pii]
Lauber, M. A., Running, W. E., & Reilly, J. P. (2009). B. subtilis ribosomal proteins: Structural homology and post-translational modifications. Journal of Proteome Research, 8(9), 4193-4206. doi:10.1021/pr801114k
Leach, K. L., Swaney, S. M., Colca, J. R., McDonald, W. G., Blinn, J. R., Thomasco, L. M., . . . Mankin, A. S. (2007). The site of action of oxazolidinone antibiotics in living bacteria and in human mitochondria. Molecular Cell, 26(3), 393-402. doi:10.1016/j.molcel.2007.04.005
Lecompte, O., Ripp, R., Thierry, J. C., Moras, D., & Poch, O. (2002). Comparative analysis of ribosomal proteins in complete genomes: An example of reductive evolution at the domain scale. Nucleic Acids Research, 30(24), 5382-5390.
Lee, C. Y., Buranen, S. L., & Ye, Z. H. (1991). Construction of single-copy integration vectors for staphylococcus aureus. Gene, 103(1), 101-105.
Li, N., Chen, Y., Guo, Q., Zhang, Y., Yuan, Y., Ma, C., . . . . Gao, N. (2013). Cryo-EM structures of the late-stage assembly intermediates of the bacterial 50S ribosomal subunit. Nucleic Acids Research, 41(14), 7073-7083. doi:10.1093/nar/gkt423 [doi]
MacKerell, A. D., Bashford, D., Bellott, M., Dunbrack, R. L., Evanseck, J. D., Field, M. J., . . . Karplus, M. (1998). All-atom empirical potential for molecular modeling and dynamics studies of proteins. The Journal of Physical Chemistry. B, 102(18), 3586-3616. doi:10.1021/jp973084f [doi]
Maguire, B. A., Beniaminov, A. D., Ramu, H., Mankin, A. S., & Zimmermann, R. A. (2005). A protein component at the heart of an RNA machine: The importance of protein 127 for the function of the bacterial ribosome. Molecular Cell, 20(3), 427-435. doi:10.1016/j.molcel.2005.09.009
Maguire, B. A., Manuilov, A. V., & Zimmermann, R. A. (2001). Differential effects of replacing escherichia coli ribosomal protein L27 with its homologue from aquifex aeolicus. Journal of Bacteriology, 183(22), 6565-6572. doi:10.1128/JB.183.22.6565-6572.2001 [doi]
Martin, A. C., Lopez, R., & Garcia, P. (1998). Pneumococcal bacteriophage cp-1 encodes its own protease essential for phage maturation. Journal of Virology, 72(4), 3491-3494.
Mikulik, K., Bobek, J., Zikova, A., Smetakova, M., & Bezouskova, S. (2011). Phosphorylation of ribosomal proteins influences subunit association and translation of poly (U) in streptomyces coelicolor. Molecular bioSystems, 7(3), 817-823. doi:10.1039/c0mb00174k; 10.1039/c0mb00174k
Minamino, T. (2014). Protein export through the bacterial flagellar type III export pathway. Biochimica Et Biophysica Acta, 1843(8), 1642-1648. doi:10.1016/j.bbamcr.2013.09.005 [doi]
Neron, B., Menager, H., Maufrais, C., Joly, N., Maupetit, J., Letort, S., . . . Letondal, C. (2009). Mobyle: A new full web bioinformatics framework. Bioinformatics (Oxford, England), 25(22), 3005-3011. doi:10.1093/bioinformatics/btp493; 10.1093/bioinformatics/btp493
Netterling, S., Vaitkevicius, K., Nord, S., & Johansson, J. (2012). A listeria monocytogenes RNA helicase essential for growth and ribosomal maturation at low temperatures uses its C terminus for appropriate interaction with the ribosome. Journal of Bacteriology, 194(16), 4377-4385. doi:10.1128/JB.00348-12 [doi]
Nierhaus, K. H. (1991). The assembly of prokaryotic ribosomes. Biochimie, 73(6), 739-755. doi:0300-9084(91)90054-5 [pii]
Nierhaus, K. H., & Dohme, F. (1974). Total reconstitution of functionally active 50S ribosomal subunits from escherichia coli. Proceedings of the National Academy of Sciences of the United States of America, 71(12), 4713-4717.
Nissen, P., Hansen, J., Ban, N., Moore, P. B., & Steitz, T. A. (2000). The structural basis of ribosome activity in peptide bond synthesis. Science (New York, N.Y.), 289(5481), 920-930. doi:8743 [pii]
O'Farrell, H. C., & Rife, J. P. (2012). Staphylococcus aureus and escherichia coli have disparate dependences on KsgA for growth and ribosome biogenesis. BMC Microbiology, 12, 244-2180-12-244. doi:10.1186/1471-2180-12-244; 10.1186/1471-2180-12-244
Oh, B., Moyer, C. L., Hendrix, R. W., & Duda, R. L. (2014). The delta domain of the HK97 major capsid protein is essential for assembly. Virology, 456-457, 171-178. doi:10.1016/j.virol.2014.03.022 [doi]
Olsson, M. H. M., Sondergaard, C., R., Rostkowski, M., & Jensen, J. H. (2011). PROPKA3: Consistent treatment of internal and surface residues in empirical pKa predictions doi:10.1021/ct100578z
Pettersen, E. F., Goddard, T. D., Huang, C. C., Couch, G. S., Greenblatt, D. M., Meng, E. C., & Ferrin, T. E. (2004). UCSF chimera—a visualization system for exploratory research and analysis. Journal of Computational Chemistry, 25(13), 1605-1612. doi:10.1002/jcc.20084
Phillips, J. C., Braun, R., Wang, W., Gumbart, J., Tajkhorshid, E., Villa, E., . . . Schulten, K. (2005). Scalable molecular dynamics with NAMD. Journal of Computational Chemistry, 26(16), 1781-1802. doi:10.1002/jcc.20289 [doi]
Poliakov, A., Chang, J. R., Spilman, M. S., Damle, P. K., Christie, G. E., Mobley, J. A., & Dokland, T. (2008). Capsid size determination by staphylococcus aureus pathogenicity island SaPI1 involves specific incorporation of SaPI1 proteins into procapsids. Journal of Molecular Biology, 380(3), 465-475. doi:10.1016/j.jmb.2008.04.065; 10.1016/j.jmb.2008.04.065
Polikanov, Y. S., Steitz, T. A., & Innis, C. A. (2014). A proton wire to couple aminoacyl-tRNA accommodation and peptide-bond formation on the ribosome. Nature Structural & Molecular Biology, 21(9), 787-793. doi:10.1038/nsmb.2871 [doi]
Rawlings, N. D., Barrett, A. J., & Bateman, A. (2011). Asparagine peptide lyases: A seventh catalytic type of proteolytic enzymes. The Journal of Biological Chemistry, 286(44), 38321-38328. doi:10.1074/jbc.M111.260026 [doi]
Redko, Y., & Condon, C. (2010). Maturation of 23S rRNA in bacillus subtilis in the absence of mini-III. Journal of Bacteriology, 192(1), 356-359. doi:10.1128/JB.01096-09 [doi]
Rossi, F., Diaz, L., Wollam, A., Panesso, D., Zhou, Y., Rincon, S., . . . Arias, C. A. (2014). Transferable vancomycin resistance in a community-associated MRSA lineage. The New England Journal of Medicine, 370(16), 1524-1531. doi:10.1056/NEJMoa1303359 [doi]
Roy, A., Kucukural, A., & Zhang, Y. (2010). I-TASSER: A unified platform for automated protein structure and function prediction. Nature Protocols, 5(4), 725-738. doi:10.1038/nprot.2010.5; 10.1038/nprot.2010.5
Sagan, L. (1967). On the origin of mitosing cells. Journal of Theoretical Biology, 14(3), 255-274.
Sali, A., Potterton, L., Yuan, F., van Vlijmen, H., & Karplus, M. (1995). Evaluation of comparative protein modeling by MODELLER. Proteins, 23(3), 318.
Sambrook, J., Fritsch, E. F., & Maniatis, T. (1989). Molecular cloning: A laboratory manual (2nd ed.). Cold Spring Harbor, N.Y.: Cold Spring Harbor Laboratory Press.
Scherl, A., Francois, P., Bento, M., Deshusses, J. M., Charbonnier, Y., Converset, V., . . . Schrenzel, J. (2005). Correlation of proteomic and transcriptomic profiles of staphylococcus aureus during the post-exponential phase of growth. Journal of Microbiological Methods, 60(2), 247-257. doi:10.1016/j.mimet.2004.09.017
Selmer, M., Dunham, C. M., Murphy, F. V., 4th, Weixlbaumer, A., Petry, S., Kelley, A. C., . . . Ramakrishnan, V. (2006). Structure of the 70S ribosome complexed with mRNA and tRNA. Science (New York, N.Y.), 313(5795), 1935-1942. doi:10.1126/science.1131127
Shajani, Z., Sykes, M. T., & Williamson, J. R. (2011). Assembly of bacterial ribosomes. Annual Review of Biochemistry, 80, 501-526. doi:10.1146/annurev-biochem-062608-160432; 10.1146/annurev-biochem-062608-160432
Shin, D. H., Lou, Y., Jancarik, J., Yokota, H., Kim, R., & Kim, S. H. (2005). Crystal structure of TM1457 from thermotoga maritima. Journal of Structural Biology, 152(2), 113-117. doi:10.1016/j.jsb.2005.08.008
Shoji, S., Dambacher, C. M., Shajani, Z., Williamson, J. R., & Schultz, P. G. (2011). Systematic chromosomal deletion of bacterial ribosomal protein genes. Journal of Molecular Biology, 413(4), 751-761. doi:10.1016/j.jmb.2011.09.004; 10.1016/j.jmb.2011.09.004
Spilman, M. S., Damle, P. K., Dearborn, A. D., Rodenburg, C. M., Chang, J. R., Wall, E. A., . . . Dokland, T. (2012). Assembly of bacteriophage 80alpha capsids in a staphylococcus aureus expression system. Virology, 434(2), 242-250. doi:10.1016/j.virol.2012.08.031; 10.1016/j.virol.2012.08.031
Sykes, M. T., Shajani, Z., Sperling, E., Beck, A. H., & Williamson, J. R. (2010). Quantitative proteomic analysis of ribosome assembly and turnover in vivo. Journal of Molecular Biology, 403(3), 331-345. doi:10.1016/j.jmb.2010.08.005 [doi]
Tejedor, F., & Ballesta, J. P. (1986). Reaction of some macrolide antibiotics with the ribosome. labeling of the binding site components. Biochemistry, 25(23), 7725-7731.
Trobro, S., & Aqvist, J. (2008). Role of ribosomal protein L27 in peptidyl transfer. Biochemistry, 47(17), 4898-4906. doi:10.1021/bi8001874; 10.1021/bi8001874
Vlieghe, P., Lisowski, V., Martinez, J., & Khrestchatisky, M. (2010). Synthetic therapeutic peptides: Science and market. Drug Discovery Today, 15(1-2), 40-56. doi:10.1016/j.drudis.2009.10.009 [doi]
Voorhees, R. M., Weixlbaumer, A., Loakes, D., Kelley, A. C., & Ramakrishnan, V. (2009). Insights into substrate stabilization from snapshots of the peptidyl transferase center of the intact 70S ribosome. Nature Structural & Molecular Biology, 16(5), 528-533. doi:10.1038/nsmb.1577; 10.1038/nsmb.1577
Wall, E. A., Caufield, J. H., Lyons, C. E., Manning, K. A., Dokland, T., & Christie, G. E. (2015). Specific N-terminal cleavage of ribosomal protein L27 in staphylococcus aureus and related bacteria. Molecular Microbiology, 95(2), 258-269. doi:10.1111/mmi.12862 [doi]
Wang, H., Takemoto, C. H., Murayama, K., Sakai, H., Tatsuguchi, A., Terada, T., . . . Yokoyama, S. (2004). Crystal structure of ribosomal protein L27 from thermus thermophilus HB8. Protein Science: A Publication of the Protein Society, 13(10), 2806-2810. doi:10.1110/ps.04864904
Wang, J., Dasgupta, I., & Fox, G. E. (2009). Many nonuniversal archaeal ribosomal proteins are found in conserved gene clusters. Archaea (Vancouver, B.C.), 2(4), 241-251.
Wang, L., Trawick, J. D., Yamamoto, R., & Zamudio, C. (2004). Genome-wide operon prediction in staphylococcus aureus. Nucleic Acids Research, 32(12), 3689-3702. doi:10.1093/nar/gkh694
Wang, Y., & Xiao, M. (2012). Role of the ribosomal protein L27 revealed by single-molecule FRET study. Protein Science: A Publication of the Protein Society, 21(11), 1696-1704. doi:10.1002/pro.2149; 10.1002/pro.2149
Weinstock, M. T., Francis, J. N., Redman, J. S., & Kay, M. S. (2012). Protease-resistant peptide design-empowering nature's fragile warriors against HIV. Biopolymers, 98(5), 431-442. doi:10.1002/bip.22073 [doi]
Williamson, J. R. (2008). Biophysical studies of bacterial ribosome assembly. Current Opinion in Structural Biology, 18(3), 299-304. doi:10.1016/j.sbi.2008.05.001 [doi]
Wilson, D. N., & Nierhaus, K. H. (2007). The weird and wonderful world of bacterial ribosome regulation. Critical Reviews in Biochemistry and Molecular Biology, 42(3), 187-219. doi:10.1080/10409230701360843
Wilson, D. N., Schluenzen, F., Harms, J. M., Starosta, A. L., Connell, S. R., & Fucini, P. (2008). The oxazolidinone antibiotics perturb the ribosomal peptidyl-transferase center and effect tRNA positioning. Proceedings of the National Academy of Sciences of the United States of America, 105(36), 13339-13344. doi:10.1073/pnas.0804276105; 10.1073/pnas.0804276105
Wolz, C., Geiger, T., & Goerke, C. (2010). The synthesis and function of the alarmone (p)ppGpp in firmicutes. International Journal of Medical Microbiology: IJMM, 300(2-3), 142-147. doi:10.1016/j.ijmm.2009.08.017 [doi]
Wower, I. K., Wower, J., & Zimmermann, R. A. (1998). Ribosomal protein L27 participates in both 50 S subunit assembly and the peptidyl transferase reaction. The Journal of Biological Chemistry, 273(31), 19847-19852.
Wower, J., Hixson, S. S., & Zimmermann, R. A. (1989). Labeling the peptidyltransferase center of the escherichia coli ribosome with photoreactive tRNA(phe) derivatives containing azidoadenosine at the 3′ end of the acceptor arm: A model of the tRNA-ribosome complex. Proceedings of the National Academy of Sciences of the United States of America, 86(14), 5232-5236.
Wower, J., Kirillov, S. V., Wower, I. K., Guven, S., Hixson, S. S., & Zimmermann, R. A. (2000). Transit of tRNA through the escherichia coli ribosome. cross-linking of the 3′ end of tRNA to specific nucleotides of the 23 S ribosomal RNA at the A, P, and E sites. The Journal of Biological Chemistry, 275(48), 37887-37894. doi:10.1074/jbc.M005031200
Wower, J., Wower, I. K., Kirillov, S. V., Rosen, K. V., Hixson, S. S., & Zimmermann, R. A. (1995). Peptidyl transferase and beyond. Biochemistry and Cell Biology=Biochimie Et Biologie Cellulaire, 73(11-12), 1041-1047.
Xiao, M., & Wang, Y. (2012). L27-tRNA interaction revealed by mutagenesis and pH titration. Biophysical Chemistry, 167, 8-15. doi:10.1016/j.bpc.2012.04.003; 10.1016/j.bpc.2012.04.003
Yonath, A. (2005). Antibiotics targeting ribosomes: Resistance, selectivity, synergism and cellular regulation. Annual Review of Biochemistry, 74, 649-679. doi:10.1146/annurev.biochem.74.082803.133130
Yusupov, M. M., Yusupova, G. Z., Baucom, A., Lieberman, K., Earnest, T. N., Cate, J. H., & Noller, H. F. (2001). Crystal structure of the ribosome at 5.5 A resolution. Science (New York, N.Y.), 292(5518), 883-896. doi:10.1126/science. 1060089 [doi]
Yutin, N., PuigbÃ², P., Koonin, E. V., & Wolf, Y. I. (2012). Phylogenomics of prokaryotic ribosomal proteins. Plos One, 7(5), e36972. Retrieved from http://dx.doi.org/10.1371%2Fjournal.pone.0036972

We claim:

1. A method of inhibiting the growth of bacteria comprising the step of exposing said bacteria to an effective amount of at least one inhibitor of L27 cleavage, wherein said bacteria comprises a consensus sequence X₁-X₂-Q-X₃-X₄-A-X₅-K-K (SEQ ID NO:17), wherein X₁ is N or D, X₂ is L or I, X₃ is L, F, or H, X₄ is L or F, and X₅ is H, S, or T.

2. The method of claim 1, wherein said at least one inhibitor is a Prp inhibitor.

3. The method of claim 2, wherein said Prp inhibitor is a dominant negative inhibitor, a protease inhibitor, a competitive inhibitor, a non-competitive inhibitor, a covalent inhibitor, a conformational inhibitor, or an uncompetitive inhibitor.

4. The method of claim 3, wherein said dominant negative inhibitor is Prp C34S or Prp C34A.

5. The method of claim 3, wherein the competitive inhibitor is a substrate analog.

6. The method of claim 5, wherein the substrate analog has an affinity more than one order of magnitude greater than L27.

7. The method of claim 5, wherein the wherein the substrate analog comprises a non-hydrolyzable peptidomimetic.

8. The method of claim 7, wherein the non-hydrolyzable peptidomimetic further comprises one or more modified amino acid residues, beta-linked amino acids, and D-amino acids.

9. The method of claim 3, wherein said covalent inhibitor comprises an alpha-ketoheterocycle covalent modification group.

10. The method of claim 1, wherein said bacteria are present in a non-human animal.

11. The method of claim 1, wherein said bacteria are present in a human.

12. The method of claim 1, wherein said step of exposing is carried out by intravenous, cutaneous, oral, intraperitoneal, intrathecal, or inhaled administration.

13. The method of claim 1, wherein said bacteria are a pathogenic organism selected from the group consisting of Staphylococcus, Bacillus, Clostridium, Listeria, Streptococcus, Mycoplasma, Fusobacteria and Enterococcus.

14. A composition comprising an inhibitor of L27 cleavage and at least one antibiotic that is different from said inhibitor.

15. A composition comprising Prp C34S or Prp C34A and a pharmaceutically acceptable carrier.

16. An isolated peptide comprising a sequence selected from the group consisting of SEQ ID NOs: 1-18 or a fragment thereof conjugated to a fluorophore and a quencher.

17. The isolated peptide of claim 16, wherein said sequence is SEQ ID NO:1.

18. The isolated peptide of claim 16, wherein the fluorophore is conjugated to the N-terminus.

19. The isolated peptide of claim 18, wherein the fluorophore comprises 2-amino benzoic acid.

20. The isolated peptide of claim 16, wherein the quencher is conjugated to the C-terminus.

21. The isolated peptide of claim 20, wherein the quencher is dinotrophenol.

22. A method for assaying Prp enzyme activity comprising the steps of
contacting Prp with an isolated peptide comprising a sequence selected from the group consisting of SEQ ID NOs: 1-18 or a fragment thereof conjugated to a fluorophore and a quencher;
measuring the level of fluorescence; and
determining Prp enzyme activity based on the measured level of fluorescence.

23. A kit comprising an isolated peptide comprising a sequence selected from the group consisting of SEQ ID NOs: 1-18 or a fragment thereof conjugated to a fluorophore and a quencher.

24. The kit according to claim 23, further comprising instructions for use and packaging.

25. A method of screening for compounds that inhibit Prp comprising the steps of
adding a test compound to a solution comprising Prp and an isolated peptide comprising a sequence selected from the group consisting of SEQ ID NOs: 1-18 or a fragment thereof conjugated to a fluorophore and a quencher;
measuring the level of fluorescence;
comparing the level of fluorescence to a reference value; and
determining the extent of Prp inhibition based on said comparison.


SEQUENCE LISTING (40 sequences)

SEQ ID NO: 1 - protein, 11 aa, Artificial Sequence
  Synthetic peptide
KLNLQFFASKK

SEQ ID NO: 2 - protein, 13 aa, Artificial Sequence
  Synthetic peptide
MLRLDLQFFASKK

SEQ ID NO: 3 - protein, 13 aa, Artificial Sequence
  Synthetic peptide
MLNMNLQLLASKK

SEQ ID NO: 4 - protein, 13 aa, Artificial Sequence
  Synthetic peptide
MLKMNLQLFAHKK

SEQ ID NO: 5 - protein, 13 aa, Artificial Sequence
  Synthetic peptide
MLLMNLQLFATKK

SEQ ID NO: 6 - protein, 14 aa, Artificial Sequence
  Synthetic peptide
MLLTMNLQLFAHKK

SEQ ID NO: 7 - protein, 14 aa, Artificial Sequence
  Synthetic peptide
MLLSMNLQLFAHKK

SEQ ID NO: 8 - protein, 14 aa, Artificial Sequence
  Synthetic peptide
MKFVLDIQLFASKK

SEQ ID NO: 9 - protein, 14 aa, Artificial Sequence
  Synthetic peptide
MQFLFNIQLFAHKK

SEQ ID NO: 10 - protein, 13 aa, Artificial Sequence
  Synthetic peptide
MLKFDIQHFAHKK

SEQ ID NO: 11 - protein, 19 aa, Artificial Sequence
  Synthetic peptide
MNNKYFLTKIDLQFFASKK

SEQ ID NO: 12 - protein, 13 aa, Artificial Sequence
  Synthetic peptide
MLKMNLQLLASKK

SEQ ID NO: 13 - protein, 13 aa, Artificial Sequence
  Synthetic peptide
MLKLNLQFFASKK

SEQ ID NO: 14 - protein, 16 aa, Artificial Sequence
  Synthetic peptide
MLKMTLNNLQLFAHKK

SEQ ID NO: 15 - protein, 16 aa, Artificial Sequence
  Synthetic peptide
MLKMNLANLQLFAHKK

SEQ ID NO: 16 - protein, 17 aa, Artificial Sequence
  Synthetic peptide
MNKLYWLTDLQLFASKK

SEQ ID NO: 17 - protein, 9 aa, Artificial Sequence
  Synthetic peptide - consensus sequence (X1 is N or D, X2 is L or I, X3 is L, F, or H, X4 is L or F, and X5 is H, S, or T)
XXQXXAXKK

SEQ ID NO: 18 - protein, 4 aa, Artificial Sequence
  Synthetic peptide - X is S or H
AXKK

SEQ ID NO: 19 - protein, 26 aa, Artificial Sequence
  Synthetic peptide
MAHKKAGGSTRNGRDSEAKRLGVKRF

SEQ ID NO: 20 - protein, 34 aa, Artificial Sequence
  Synthetic peptide
MLRLDLQFFASKKGVGSTKNGRDSEAKRLGAKRA

SEQ ID NO: 21 - protein, 34 aa, Artificial Sequence
  Synthetic peptide
MLKLNLQFFASKKGVSSTKNGRDSESKRLGAKRA

SEQ ID NO: 22 - protein, 38 aa, Artificial Sequence
  Synthetic peptide
MEENKLKFNLQFFADQSDDPDEPGGDGKKGNPDKKEND

SEQ ID NO: 23 - protein, 35 aa, Artificial Sequence
  Synthetic peptide
MEQTQKLKLNLQHFASNNVKPQVFNPDNVMMHEKK

SEQ ID NO: 24 - protein, 106 aa, Staphylococcus aureus
MITVDITVNDEGKVTDVIMDGHADHGEYGHDIVCAGASAVLFGSVNAIIGLTSERPDINYDDNGGHFHIRSVDTNNDEAQLILQTMLVSLQTIEEEYNENIRLNYK

SEQ ID NO: 25 - protein, 112 aa, Bacillus subtilis
MIQATIRRSHDKGILSFEMTGHANFAEHGQDLVCAGVTAVVFGAVNAVIVLAGFEPLLDIGEDGGYFYFEFPESLDPEARQKAQLLIEGMIVSLETIERDYKDNLRVTTNII

SEQ ID NO: 26 - protein, 114 aa, Streptococcus pneumoniae
MIQAVFERAEDGELRSAEITGHAESGEYGLDVVCASVSTLAINFINSIEKFAGYEPILELNEDEGGYLMVEIPKDLPSHQREMTQLFFESFFLGMANLSENSSEFVQTRVITEN

SEQ ID NO: 27 - protein, 111 aa, Streptococcus mutans
MIQATFIRRKGILESVELIGHAGSGEYGFDIVCAAVSTLSMNLVNALEVLADCTVSLQMDEFDGGYMKIDLSYITNKSDEKVQLLFEAFLLGITNLAENSPEFVTAKIMTQ

SEQ ID NO: 28 - protein, 108 aa, Aminobacterium colombiense
MTIITITRQSGEIVSVEAMGHSGYAKAGSDIVCAGISALVQALAVGIKEVACVEKASFLVDEENATIKIWWPRTEKKEIQVLAETIVLSLKTISESYAGYVKFMEVRK

SEQ ID NO: 29 - protein, 109 aa, Fusobacterium nucleatum
MTKVEIFRKNGSIVGYKASGHSGYSEQGSDIICSAIATSLQMTLAGIQEVLKLEPKFKINDGFLDVDLKDISQNKFTEINILTESMALFLRELAKQYPKYIRLVEKEEK

SEQ ID NO: 30 - protein, 112 aa, Thermotoga lettingae
MIKIVFYTFKGGYSGFKINGHSNFDVKGKDIVCAAISALSQHTAKMLVKHCGARIVARQEGLLEVALNNIDNFSNVVLSEFKESIEDLQSQYPRNISLEVKEDANRHTVVCS

SEQ ID NO: 31 - protein, 100 aa, Mycoplasma pneumoniae
MIKLTVSHHKLTASGHALFAKKGQDIVCAAVSGIIFGALPWFETNSIAVQEDATVPSLSLELVQPTAKLITGLSVVIMQLKTLAHSYPQFISFEDQRKDE

SEQ ID NO: 32 - protein, 13 aa, Artificial Sequence
  Synthetic peptide
MLKLNLQFFASKK

SEQ ID NO: 33 - protein, 5 aa, Artificial Sequence
  Synthetic peptide
MASKK

SEQ ID NO: 34 - protein, 13 aa, Artificial Sequence
  Synthetic peptide
MLKLNLQAAASKK

SEQ ID NO: 35 - protein, 9 aa, Artificial Sequence
  Synthetic peptide
MLKLNLQFF

SEQ ID NO: 36 - protein, 93 aa, Thermotoga maritima
MIKVTVTNSFFEVTGHAPDKTLCASVSLLTQHVANFLKAEKKAKIKKESGYLKVKFEELENCEVKVLAAMVRSLKELEQKFPSQIRVEVIDNG

SEQ ID NO: 37 - protein, 4 aa, Artificial Sequence
  Synthetic peptide
MLKL

SEQ ID NO: 38 - protein, 7 aa, Artificial Sequence
  Synthetic peptide
MNLQLLA

SEQ ID NO: 39 - protein, 7 aa, Artificial Sequence
  Synthetic peptide
INLSLCA

SEQ ID NO: 40 - protein, 11 aa, Artificial Sequence
  Synthetic peptide
PVLEGARINHA